(19) United States
(12) Patent Application Publication
(10) Pub. No.: US 2005/0232931 A1
(43) Pub. Date: Oct. 20, 2005

(54) Preparation and application of anti-tumor bifunctional fusion proteins

(75) Inventors: Jing Ma, Laguna Niguel, CA (US); Yanjun Guo, Shanghai (CN)

Correspondence Address: FOLEY & LARDNER, P.O. BOX 80278, SAN DIEGO, CA, 92138-0278, US

(73) Assignee: Oncomax Acquisition Corp.

(21) Appl. No.: 11/004,639

(22) Filed: Dec. 2, 2004

Related U.S. Application Data
(63) Continuation-in-part of application No. 10/723,003, filed on Nov. 26, 2003.

(30) Foreign Application Priority Data
Jun. 13, 2003 (CN) 03129290.9
Nov. 25, 2003 (CN) 200310119930.0

(51) Int. Cl. A61K 39/00; C07K 14/82

(52) U.S. Cl. 424/185.1; 530/350

(57) ABSTRACT

Provided herein is a chimeric protein, which chimeric protein comprises a Flt3 ligand, or a biologically active fragment thereof, and a proteinaceous or peptidyl tumoricidal agent, or a targeting agent which binds to a receptor expressed on a tumor, and uses thereof, particularly in the treatment of malignancy. Other embodiments and uses are disclosed.

### CROSS-REFERENCE TO RELATED APPLICATIONS

This application is a continuation-in-part of U.S. application Ser. No. 10/723,003, filed Nov. 26, 2003, which claims the benefit of PRC application serial no. 03129290.9, filed Jun. 13, 2003, and PRC application 200310119930.0, filed Nov. 25, 2003 (title: Preparation and application of anti-tumor bifunctional fusion proteins), all of which are incorporated herein in their entirety including the drawings by reference thereto.

### BACKGROUND OF THE INVENTION

This invention relates to the field of tumor immunology, mainly about the anti-tumor bifunctional fusion proteins and their nucleic acid sequences, methods of preparation and application of them in preparation of antitumor drugs.

Tumor immunotherapy involves the induction of tumor regression by modulation of natural host defense mechanisms or by manipulation with a immunological agent. Immunotherapy is a recognized therapeutic modality for the treatment of malignancies along with the traditional modalitiies of surgical resection, radiotherapy and chemotherapy. In fact, immunotherapy is sometimes used as “complementary therapy” for the more common therapies such as surgery and radiation. The impetus for such combination therapy lies in the shortcomings in traditional modalities. For example, in China, liver cancer, breast cancer and lymphoma are the most commonly occurring cancers. However, two thirds of hepatoma patients have inoperable tumor burdens at the time of diagnosis. More importantly, even if the modality of surgical resection is available to such patients, the problem of distant, undetected micrometastases remains untreated by such therapy. Likewise, the traditional therapies of radiotherapy and chemotherapy also have significant limitations, most prominently the systemic inhibition of the hematopoietic and immune system. Thus, the toxic effects of radiotherapy and chemotherapy limit efficacy of these therapies in the cases where radical treatment is most desired—in the patient with significant tumor burden at the time of diagnosis. Therefore, it is desirable to find novel effective strategies that will complement traditional therapies.

Immunotherapy of tumors can be effected through the administration of antibodies specific for tumor antigens. While antibodies typically have been used as delivery agents for toxic moieties, recent studies indicated that the monoclonal antibodies (mAbs) against certain cell surface molecules, e.g., FAS, EGFR, and HER2, directly induced tumor cell death through the triggering of apoptotic pathways. See, e.g., Shimizu et al., Biochem. Biophys. Res. Commun. 228(2):375-79 (1996). This suggests that the modulation of particular signaling pathways, particularly those resulting in tumor cell death, may provide a successful strategy for antibody-mediated tumor immunotherapy. At least one antibody employing this strategy has been successful during clinical trials. Herceptin, a monoclonal antibody specific for human HER2, induces apoptosis in Her2⁺ tumor cells and has been used successfully for the in vivo treatment of breast cancer. See e.g., Burstein et al., J. Clin. Oncol. 21:2889-95 (2003). However, one of the recognized limitations of such antibody therapy is the likelihood that distant metastases may still escape such therapy or that antigen-negative variants will develop, leading to a later relapse with metastatic disease.

Immunotherapy can also be effected through the elicitation of an active anti-tumor immune response in the patient following the administration of a tumor vaccine. Ideally, the tumor vaccine delivers immunogenic tumor antigens to suitable antigen presenting cells, resulting in the generation of an effective and long-lasting anti-tumor immune response. Studies have demonstrated that the dendritic cell (DC), a type of antigen presenting cell, plays a crucial role in an effective anti-tumor immune response. See e.g., Zitvogel et al., J. Exp. Med. 183:87-97 (1996); Choudhury et al., Blood 89:1133-42 (1997); and DiNicola et al., Cytokines Cell Mol. Therapy 4:265-73 (1998). DCs stimulate the differentiation of naive CD4+ and CD8+ T cells to T helper cells (Th) and cytotoxic T lymphocytes (CTLs), respectively. DCs can express high levels of both class I and class II major histocompatibility complex (MHC) antigens, costimulatory molecules, adhesion molecules and secrete high levels of IL-12, a potent cytokine in CTL differentiation and activation. See e.g., Banchereau et al., Nature 392:245-52 (1998); Banchereau et al., Ann. Rev. Immunol. 18:767-811 (2000). As the CTL-mediated anti-tumor response is believed to generate long term protection against tumor regrowth, DCs appear to be the antigen presenting cell of choice for tumor immunotherapy.

While tumor vaccines clearly confer long term protection against tumor metastatic outgrowth and even subsequent tumor challenges, the clinical application of this knowledge has proved to be difficult. See e.g., Fong et al., Ann. Rev. Immunol. 18:245-73 (2000). First, it has proven difficult to reliably expand functional DCs in ex vivo expansion protocols. Because the immune is necessarily MHC-restricted, any ex vivo DCs employed in an immunotherapy strategy must be the DCs of the patient being treated. Second, reproducible activation of DCs in vivo has not yet been achieved. Third, no clear protocol has been established that permits the activation and antigen loading of the desired DC population, i.e., those capable of eliciting an anti-tumor response. In sum, the expansion of activated DCs selectively located at tumor site that present immunogenic tumor antigens is a problem that remains unsolved.

Therefore, while it is clear that immune molecules, e.g., tumor-specific antibodies, and vaccines eliciting immune responses can effect tumor growth, a unified approach that permits the simultaneous reduction of tumor growth and the generation of lasting protective immune response is still lacking.

### BRIEF SUMMARY OF THE INVENTION

Provided herein is a chimeric protein that permits the simultaneous eradication of tumor cells and the stimulation of an effective anti-tumor immune response. Specifically, the chimeric protein comprises at least two components. The first component is Flt3 ligand (FL), or a biologically active fragment thereof. FL is a potent chemotactic molecule and activator for DCs and other anti-tumor effectors such as NK cells. The second component is a tumoricidal agent that induce cell death. Such agents can be a ligand or a tumor-specific antibody that induces apoptosis directly, i.e., through the direct initiation of the apoptotic cascade (e.g., Fas ligand), or a tumor-specific antibody that mediates apoptosis indirectly, i.e., through cytokine deprivation related-apoptosis (e.g., anti-EGFR antibody). While not wishing to be bound by any theory, it is believed that the chimeric protein reduces tumor burden by directly inducing the apoptosis of tumor cells while also targeting and activating DCs, and other antitumor effectors, e.g., NK cells, to infiltrate the tumor tissues. Tumor antigens released by the dying tumor cells then can be processed and presented by FL-activated DCs, that then effectively serve as antigen-presenting cells for a specific anti-tumor immune response. Therefore, the chimeric proteins of the invention simultaneously effect direct and indirect tumor cell elimination while eliciting an effective active immune response against the tumor cells that prevents the recurrence of tumor growth.

In one aspect, the present invention is directed to a isolated chimeric protein, which chimeric protein comprises a Flt3 ligand, or a biologically active fragment thereof, and a proteinaceous or peptidyl tumoricidal agent.

In another aspect, the present invention is directed to an isolated nucleic acid encoding a chimeric protein, which chimeric protein comprises a Flt3 ligand, or a biologically active fragment thereof, and a proteinaceous or peptidyl tumoricidal agent, wherein the agent is other than TRAIL. Recombinant cell comprising the nucleic acid and methods for producing the chimeric protein using the nucleic acid are also provided.

In yet another aspect, the present invention is directed to a pharmaceutical composition comprising an effective amount of an isolated chimeric protein comprising a Flt3 ligand and a proteinaceous or peptidyl tumoricidal agent, and a pharmaceutically acceptable carrier or excipient.

In some embodiments of the invention, the amino acid sequences of the chimeric proteins and the nucleotide sequences encoding the chimeric proteins comprise the sequences shown in FIGS. 16-18, 20-22, 27-29, 35-37, and 39 and 41-42. CHO cells containing nucleic acid encoding a form of ReSM5-1 containing from the N-terminus, the signal sequence and extracellular domain of flt-3 ligand, a hinge domain from human IgG γ1, a CH₂, and CH₃ domain from human γ1, and a single chain Fv form of antibody ReSM5-1, containing from the humanized variable regions of the antibody connected by a flexible linker has been deposited with the American Type Culture Collection (ATCC) on Nov. 23, 2004 under accession number PTA-6327. This construct is shown in FIG. 18.

In a further aspect, the present invention is directed to a combination, which combination comprises: a) an effective amount of a chimeric protein comprising a Flt3 ligand and a proteinaceous or peptidyl tumoricidal agent; and b) an effective amount of an anti-neoplastic agent.

In yet another aspect, the present invention is directed to a method for treating cancer in a mammal so afflicted, which method comprises administering to a mammal an effective amount of the above combination, wherein the cancer expresses a target for the proteinaceous or peptidyl tumoricidal agent.

In another aspect, the present invention is directed to a kit comprising an effective amount of a chimeric protein comprising a Flt3 ligand and a proteinaceous or peptidyl tumoricidal agent, and an instruction means for administering the chimeric protein.

In one aspect, the present invention is directed to a method for treating cancer in a mammal to afflicted, which method comprises administering to the mammal an effective amount of a chimeric protein comprising a Flt3 ligand and a proteinaceous or peptidyl tumoricidal agent, wherein the cancer expresses a target for the proteinaceous or peptidyl tumoricidal agent.

In another aspect, the present invention is directed to a method for inducing caspase-3 mediated apoptosis in a cell, which method comprises contacting the cell with an effective amount of an isolated chimeric protein comprising a Flt3 ligand and a proteinaceous or peptidyl tumoricidal agent, wherein the cell expresses a target for the proteinaceous or peptidyl tumoricidal agent.

In yet another aspect, the present invention is drawn to a vaccine comprising an effective amount of a chimeric protein comprising a Flt3 ligand and a proteinaceous or peptidyl tumoricidal agent, and an immune response potentiator other than flt3 ligand.

In another aspect, the present invention is directed to a method for eliciting an anti-cancer immune response in a mammal so afflicted, which method comprises administering to the mammal an effective amount of the vaccine disclosed herein.

In yet another aspect, the present invention is directed to a method for producing a tumor-specific lymphocyte, which method comprises administering to a mammal an effective amount of a chimeric protein comprising a Flt3 ligand and a proteinaceous or peptidyl tumoricidal agent to generate a tumor-specific lymphocyte, and recovering the generated tumor-specific lymphocyte from the mammal.

In another aspect, the present invention provides an isolated chimeric protein, which chimeric protein includes a Flt3 ligand, or a biologically active fragment thereof, and a proteinaceous or peptidyl targeting agent which binds to a receptor expressed on tumor cells other than the Fc receptor. In preferred embodiments, the tumor cell receptor agent is not the E6 or E7 protein of human papillomavirus or a receptor for TRAIL. Also provided are nucleic acids encoding this protein and various other applications which are similar to the described for other chimeric proteins of the invention discussed above.

### BRIEF DESCRIPTION OF SEVERAL VIEWS OF THE DRAWING(S)

FIG. 1 shows the structures of (A) a tetravalent bispecific antibody and a (B) FLex/Fc/Fv bifunctioiial fusion protein.

FIG. 2 shows the nucleotide sequence (SEQ ID NO:1) and amino acid sequence (SEQ ID NO:2) of the human flt3 ligand signal peptide (SP) and flt3 ligand extracellular domain (hFLex).

FIG. 3 shows the nucleotide sequence (SEQ ID NO:3) and amino acid sequence (SEQ ID NO:4) of a chimeric protein containing the human flt3 ligand signal peptide (SP) and extracellular domain and the Fc of an IgG heavy chain which includes a hinge, CH2 and CH3 domains.

FIG. 4 shows the nucleotide sequence (SEQ ID NO:5) and amino acid sequence (SEQ ID NO:6) of linker (Gly4Ser)3.

FIG. 5 shows agarose gel analysis of anti-p230 antibody (SM5-1) variable region gene PCR products on a 1% agarose gel.

FIG. 6 shows the nucleotide sequence (SEQ ID NO:7) and amino acid sequence (SEQ ID NO:8) of murine SM5-1 (“mSM5-1”) heavy chain signal peptide (SP) and heavy chain variable region (VH).

FIG. 7 shows the nucleotide sequence (SEQ ID NO:9) and amino acid sequence (SEQ ID NO:10) of mSM5-1 light chain signal peptide (SP) and light chain variable region (VL).

FIG. 8 shows the nucleotide sequence (SEQ ID NO: 11) and amino acid sequence (SEQ ID NO: 12) of a mouse/human chimeric SM5-1 heavy chain (ChSM). Signal peptide (SP); variable heavy (VH), constant heavy (CH) Stop: translation termination codon. The shaded region indicates the introns.

FIG. 9 shows the nucleotide sequence (SEQ ID NO: 13) and amino acid sequence (SEQ ID NO: 14) of a mouse/human chimeric SM5-1 light chain. Signal peptide (SP); murine variable light (VL), human constant light (CL) Stop: translation termination codon.

FIG. 10 shows the diagram of SM5-1 chimeric heavy chain expression vector. Regions of the expression vector encoding different functions are indicated: HCMV prom, human cytomegalovirus Major Immediate Early promoter; VH, the heavy chain variable region gene of huSM; CH, the human γ1 chain constant region gene. BGH pA, Bovine growth hormone polyadenylation signal; SV40 ori, simian virus 40 early promoter and origin of replication; DHFR, dihydrofolate reductase gene; pUC origin, plasmid origin of replication; Amp designates the β-lactamase gene.

FIG. 11 shows the diagram of the SM5-1 chimeric light chain expression vector. Regions of the vector encoding different functions are indicated: HCMV prom, human cytomegalovirus Major Immediate Early promoter; VL, the light chain variable region gene of huSM; CL, the human K chain constant region gene; BGH pA, Bovine growth hormone polyadenylation signal; SV40 ori, simian virus 40 early promoter and origin of replication; DHFR, dihydrofolate reductase gene; pUC origin, plasmid origin of replication; Amp designates the β-lactamase gene.

FIG. 12 shows the nucleotide sequence (SEQ ID NO: 15) and amino acid sequence (SEQ ID NO: 16) of an SM5-1 humanized antibody (huSM) signal peptide and heavy chain variable region. Signal peptide (SP); variable heavy (VH).

FIG. 13 shows the nucleotide sequence (SEQ ID NO: 17) and amino acid sequence (SEQ ID NO: 18) of an SM5-1 humanized antibody (huSM) light chain signal peptide and variable region. Signal peptide (SP); variable light (VL).

FIG. 14 shows the nucleotide sequence (SEQ ID NO:19) and amino acid sequence (SEQ ID NO:20) of the signal peptide and heavy chain of an SM5-1 humanized antibody (huSM). Signal peptide (SP); variable heavy (VH), constant heavy (CH) Stop: translation termination codon. The shaded region indicates the introns.

FIG. 15 shows the nucleotide sequence (SEQ ID NO:21) and amino acid sequence (SEQ ID NO:22) of the signal peptide and light chain of an SM5-1 humanized antibody. Signal peptide (SP); variable light (VL), constant light (CL) Stop: translation termination codon.

FIG. 16 shows the nucleotide sequence (SEQ ID NO:23) and amino acid sequence (SEQ ID NO:24) of a chimeric protein containing humanized SM5-1 signal sequence and heavy chain fused to the human flt3 ligand extracellular domain (HuSMVH/Fc/hFLex). Signal peptide (SP); Stop: translation termination codon. The shaded region indicates the introns.

FIG. 17 shows the nucleotide sequence (SEQ ID NO:25) and amino acid sequence (SEQ ID NO:26) of a chimeric protein containing humanized SM5-1 signal sequence and heavy chain and the human flt3 ligand extracellular domain connected by a flexible linker (huSMVH/Fc/Link/hFLex). Signal peptide (SP); Stop: translation termination codon. The shaded region indicates the introns.

FIG. 18 shows the nucleotide sequence (SEQ ID NO:27) and amino acid sequence (SEQ ID NO:28) of a chimeric protein containing a human flt3 signal peptide (SP) and extracellular domain fused to a human IgG Fc (hinge, CH2 and CH3) fused to a humanized SM5-1 single chain Fv fragment (hFLex/Fc/huSMFv).

FIG. 19 shows a diagrammatic representation of the chimeric protein described in FIG. 18 (FL/Fc/Fv).

FIG. 20 shows the nucleotide sequence (SEQ ID NO:29) and amino acid sequence (SEQ ID NO:30) of a chimeric protein containing a chimeric mouse/human SM5-1 heavy chain fused to the human flt3 ligand extracellular domain (chSMVH/Fc/hFLex). Signal peptide (SP); Stop: translation termination codon. The shaded region indicates the introns.

FIG. 21 shows the nucleotide sequence (SEQ ID NO:31) and amino acid sequence (SEQ ID NO:32) of chimeric protein containing a mouse/human chimeric SM5-1 heavy chain fused via a linker to the human flt3 ligand extracellular domain (chSMVH/Fc/Link/hFLex). Signal peptide (SP); Stop: translation termination codon. The shaded region indicates the introns.

FIG. 22 shows the nucleotide sequence (SEQ ID NO:33) and amino acid sequence (SEQ ID NO:34) of a chimeric protein containing human flt3 signal peptide (SP) and extracellular domain fused to a human IgG Fc (hinge, CH2 and CH3) fused to chimeric SM5-1 single chain Fv fragment (hFLex/Fc/chSMFv).

FIG. 23 shows the nucleotide sequence (SEQ ID NO:35) and amino acid sequence (SEQ ID NO:36) of 2B8 (anti-CD20) heavy chain signal peptide (SP) and variable region (VH).

FIG. 24 shows the nucleotide sequence (SEQ ID NO:37) and amino acid sequence (SEQ ID NO:38) of 2B8 (anti-CD20) light chain signal peptide (SP) and light chain variable region (VL).

FIG. 25 shows the nucleotide sequence (SEQ ID NO:39) and amino acid sequence (SEQ ID NO:40) of the heavy chain of the anti-CD20 chimeric antibody. Signal peptide (SP); Stop: translation termination codon. The shaded region indicates the introns.

FIG. 26 shows the nucleotide sequence (SEQ ID NO:41) and amino acid sequence (SEQ ID NO:42) of the light chain of the anti-CD20 chimeric antibody. Signal peptide (SP); Stop: translation termination codon.

FIG. 27 shows the nucleotide sequence (SEQ ID NO:43) and amino acid sequence (SEQ ID NO:44) of a chimeric protein containing the heavy chain of anti-CD20 antibody fused the human flt3 extracellular domain (CD20V_{H}/Fc/hFLex). Signal peptide (SP); Stop: translation termination codon. The shaded region indicates the introns.

FIG. 28 shows the nucleotide sequence (SEQ ID NO:45) and amino acid sequence (SEQ ID NO:46) of a chimeric protein containing the heavy chain of anti-CD20 antibody and the human flt3 extracellular domain connected by a flexible linker (CD20V_{H}/Fc/Link/hFLex). Signal peptide (SP); Stop: translation termination codon. The shaded region indicates the introns.

FIG. 29 shows the nucleotide sequence (SEQ ID NO:47) and amino acid sequence (SEQ ID NO:48) of a chimeric protein containing the human flt3 ligand signal peptide and extracellular domain fused to a human IgG Fc (hinge, CH2 and CH3) fused to anti-CD20 single chain Fv fragment (hFLex/Fc/CD20Fv). Signal peptide (SP); Stop: translation termination codon.

FIG. 30 shows a diagrammatic representation of the chimeric protein described in FIG. 29 (FL/Fc/Fv).

FIG. 31 shows the nucleotide sequence (SEQ ID NO:49) and amino acid sequence (SEQ ID NO:50) of the anti-HER-2 antibody signal peptide (S) and heavy chain variable region (VH).

FIG. 32 shows the nucleotide sequence (SEQ ID NO:51) and amino acid sequence (SEQ ID NO:52) of the anti-HER-2 antibody signal peptide (SP) and light chain variable region (VL).

FIG. 33 shows the nucleotide sequence (SEQ ID NO:53) and amino acid sequence (SEQ ID NO:54) of the heavy chain of the anti-HER-2 humanized antibody. Signal peptide (SP); Stop: translation termination codon. The shaded region indicates the introns.

FIG. 34 shows the nucleotide sequence (SEQ ID NO:55) and amino acid sequence (SEQ ID NO:56) of the light chain of the anti-HER-2 humanzied antibody. Signal peptide (SP); Stop: translation termination codon.

FIG. 35 shows the nucleotide sequence (SEQ ID NO:57) and amino acid sequence (SEQ ID NO:58) of a chimeric protein containing the heavy chain of the anti-HER-2 antibody fused to the human flt3 ligand extracellular domain (Her2VH/Fc/hFLex). Signal peptide (SP); Stop: translation termination codon. The shaded region indicates the introns.

FIG. 36 shows the nucleotide sequence (SEQ ID NO:59) and amino acid sequence (SEQ ID NO:60) of a chimeric protein containing the heavy chain of the anti-HER-2 antibody and the human flt3 ligand extracellular domain connected by a flexible linker (her2VH/Fc/Link/hFLex). Signal peptide (SP); Stop: translation termination codon. The shaded region indicates the introns.

FIG. 37 shows the nucleotide sequence (SEQ ID NO:61) and amino acid sequence (SEQ ID NO:62) of a chimeric protein containing the human flt3 ligand signal peptide and extracellular domain fused to a human IgG Fc (hinge, CH2 and CH3) fused to the anti-HER-2 single chain Fv fragment (hFLex/Fc/her2Fv). Signal peptide (SP); Stop: translation termination codon.

FIG. 38 shows a diagrammatic representation of the chimeric protein described in FIG. 37 (FL/Fc/Fv).

FIG. 39 shows the nucleotide sequence (SEQ ID NO:63) and amino acid sequence (SEQ ID NO:64) sequences of hFLex/Trailex. SP, signal peptide; Stop, translation termination codon.

FIG. 40 shows a diagrammatic representation of the chimeric protein described in FIG. 39 (FL/Trail).

FIG. 41 shows the nucleotide sequence (SEQ ID NO:65) and amino acid sequence (SEQ ID NO:66) of a chimeric protein containing human flt3 ligand signal peptide and extracellular domain and the TRAIL extracellular domain connected by an isoleucine zipper (hFLex/IZ/TRAILex). Signal peptide (SP); Stop: translation termination codon.

FIG. 42 shows the nucleotide sequence (SEQ ID NO:67) and amino acid sequence (SEQ ID NO:68) of a chimeric protein containing the human flt3 ligand signal peptide and extracellular domain fused to a human IgG Fc (hinge, CH2 and CH3) fused to the TRAIL extracellular domain (hFLex/Fc/TRAILex). Signal peptide (SP); Stop: translation termination codon.

FIG. 43 shows a diagrammatic representation of the chimeric protein described in FIG. 42 (FL/Fc/TRAIL).

FIG. 44 shows the effects of various chimeric proteins on expansion effects of human cord blood CD34(+) cells. FL (flt3 ligand extracellular domain); chSM (chimeric SM5-1 antibody); huSM (humanized SM5-1 antibody); ChSM/FL (FL/Fc/chSMFv); huSM/FL (FL/Fc/huSMFv).

FIG. 45 shows the effects of various chimeric proteins on NK and DC cells in vivo. FL (flt3 ligand extracellular domain); ChSM/FL (FL/Fc/chSMFv); huSM/FL (FL/Fc/huSMFv).

FIG. 46A shows the inhibitory effect of chSM/FL chimeric protein on different cell lines in vitro. ChSM/FL (FL/Fc/chSMFv).

FIG. 46B shows the inhibitory effect of chimeric protein huSM/FL on different cell lines in vitro. huSM/FL (FL/Fc/huSMFv).

FIG. 47A shows the inhibitory effect of various chimeric proteins on B16 melanoma cell proliferation in vitro. ChCD3/FL (FL/Fc/ChCD3Fv); huCD3/FL (FL/Fc/huCD3Fv); ChSM/FL (FL/Fc/chSMFv); huSM/FL (FL/Fc/huSMFv).

FIG. 47B shows the inhibitory effects of various chimeric proteins on Hepa1-6 cell proliferation in vitro. ChCD3/FL (FL/Fc/ChCD3Fv); huCD3/FL (FL/Fc/huCD3Fv); ChSM/FL (FL/Fc/chSMFv); huSM/FL (FL/Fc/huSMFv).

FIG. 47C shows the inhibitory effects of various chimeric proteins on B16/p230 cell proliferation in vitro. ChCD3/FL (FL/Fc/ChCD3Fv); huCD3/FL (FL/Fc/huCD3Fv); ChSM/FL (FL/Fc/chSMFv); huSM/FL (FL/Fc/huSMFv).

FIG. 47D shows the inhibitory effects of various chimeric proteins on Hepa 1-6/p230 cell proliferation in vitro. ChCD3/FL (FL/Fc/ChCD3Fv); huCD3/FL (FL/Fc/huCD3Fv); ChSM/FL (FL/Fc/chSMFv); huSM/FL (FL/Fc/huSMFv).

FIG. 48A shows the inhibitory effect of her2/FL (shown FL/her2) on different cell lines in vitro. her2/F1 (FL/Fc/HERFv).

FIG. 48B shows the inhibitory effect of herceptin (anti-HER-2 antibody) on different cell lines in vitro.

FIG. 49A shows the inhibitory effect of her2/FL on different cell lines in vitro. her2/Fl (FL/Fc/HER2Fv).

FIG. 49B shows the inhibitory effect of herceptin (anti-HER-2 antibody) on different cell lines in vitro.

FIG. 50 shows the inhibitory effect of CD20/FL and Rituximab in vitro. CD20/FL (FL/Fc/CD20Fv).

FIG. 51A shows the inhibitory effects of Trail/FL on different cell lines in vitro. Trail/FL (hFlex/IZ/Trailex).

FIG. 51B shows the inhibitory effects of Trail on different cell lines in vitro.

FIG. 52A shows the inhibitory effect of Trail/FL on different cell lines in vitro. Trail/FL (hFlex/IZ/Trailex).

FIG. 52B shows the inhibitory effect of Trail on different cell lines in vitro.

FIG. 53 shows the effect of her2/FL on breast cancer BT474 tumor growth in vivo. PBS (phosphate buffered saline); her2/FL (FL/Fc/Her2Fv).

FIG. 54 shows the effect of CD20/FL on Raji cell tumor growth in vivo. PBS (phosphate buffered saline); CD20/FL (FL/Fc/CD20Fv).

FIG. 55 shows the effect of Trail/FL on hepatoma QYC tumor growth in vivo. PBS (phosphate buffered saline); Trail/FL (hFlex/IZ/Trailex).

FIG. 56 shows the biodistribution of chimeric proteins injected i.v. into B16p230 tumor bearing animals. SM (chimeric SM5-1 antibody); hSM (humanized SM5-1 antibody); SM/FL (FL/Fc/chSMFv); hSM/FL (FL/Fc/huSMFv).

FIG. 57 shows the biodistribution of chimeric proteins in animals bearing 4T1/her2, A20/20 and Renca tumors. Her2 (anti-HER-2 antibody); Her2/FL (FL/Fc/Her2Fv); CD20 (anti-CD20 antibody); CD20/FL (FL/Fc/CD20Fv); TRAIL/FL (hFlex/IZ/Trailex).

### DETAILED DESCRIPTION OF THE INVENTION

For clarity of disclosure, and not by way of limitation, the detailed description of the invention is divided into the subsections that follow.

A. Definitions

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, “nucleic acid (s)” refers to deoxyribonucleic acid (DNA) and/or ribonucleic acid (RNA) in any form, including inter alia, single-stranded, duplex, triplex, linear and circular forms. It also includes polynucleotides, oligonucleotides, chimeras of nucleic acids and analogues thereof. The nucleic acids described herein can be composed of the well-known deoxyribonucleotides and ribonucleotides composed of the bases adenosine, cytosine, guanine, thymidine, and uridine, or may be composed of analogues or derivatives of these bases. Additionally, various other oligonucleotide derivatives with nonconventional phosphodiester backbones are also included herein, such as phosphotriester, polynucleopeptides (PNA), methylphosphonate, phosphorothioate, polynucleotides primers, locked nucleic acid (LNA) and the like.

As used herein, a “composition” refers to any mixture of two or more products or compounds. It may be a solution, a suspension, liquid, powder, a paste, aqueous, non-aqueous, or any combination thereof.

As used herein, a “combination” refers to any association between two or among more items.

B. Chimeric Proteins Comprising Flt3 Ligand and a Tumorical Agent, and Nucleic Acids Encoding the Same

In one aspect, the present invention is directed to a chimeric protein, which chimeric protein comprises a Flt3 ligand (“FL”), or a biologically active fragment thereof, and a proteinaceous or peptidyl tumoricidal agent. Preferably, the chimeric protein is an isolated protein, i.e., free of association with other proteins, polypeptides, or other molecules. In some embodiments, the chimeric protein is a purification product of a recombinant host cell culture or as a purified extract. An “isolated” protein or nucleic acid is at least 20% pure, more preferably at least 30%, more preferably at least 40%, more preferably at least 50%, more preferably at least 60%, more preferably at least 70%, more preferably at least 80%, more preferably at least 90%, more preferably at least 95%, and even more preferably at least 99% pure.

Any suitable Flt3 ligand can be used in the compositions and methods provided herein. As used herein, the term “Flt3 ligand” refers to a genus of polypeptides that bind and induce signaling through the Flt3 receptor found of progenitor cells. It is also intended that a Flt3 ligand, or a biologically active fragment thereof, can include conservative amino acid substitutions that do not substantially alter its activity. Suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity. See, e.g., Watson, et al., MOLECULAR BIOLOGY OF THE GENE, 4th Edition, 1987, The Benjamin/Cummings Pub. Co., p. 224. Such exemplary substitutions are preferably made in accordance with those set forth in TABLE 1 as follows:

TABLE 1

| Original residue | Conservative substitution |
|---|---|
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions are also permissible and may be determined empirically or in accord with known conservative substitutions.

Flt3 ligand is a type I transmembrane protein that can be released as a soluble homodimeric protein. See, e.g., Lyman et al., Flt3 ligand in THE CYTOKINE HANDBOOK (Thomson et al ed., 4th ed (2003)). In one embodiment, the Flt3 ligand, or a biologically active fragment thereof, is a soluble Flt3 ligand. In one embodiment of the compositions and methods provided herein, Flt3 ligand, or a biologically active fragment thereof, is a mammalian Flt3-ligand, more preferably a human Flt3-ligand. The human Flt3 ligand is 72% identical to the murine protein at the amino acid level and conserves many of the features of the murine protein, including glycosylation sites, key cysteine residues, and splice junctions. Suitable Flt3 ligand proteins include those disclosed in Lyman et al., Cell 75:1157-67 (1993), Hannum et al., Nature, 368:364-67 (1996); U.S. Pat. No. 5,843,423; U.S. Patent Application Ser. Nos: 200030113341 and 20030148516; and Genebank Accession Nos. NM 001459, U2 9874, U03858, and U04806.

The Flt3 ligand receptor, Flt3, is a member of the class III receptor tyrosine kinase (RTKIII) receptor family. In normal cells, Flt3 is expressed in immature hematopoietic cells, typically CD34+ cells, placenta, gonads, and brain. See, e.g., Rosnet, et al., Blood 82:1110-19 (1993); Small et al., Proc. Natl. Acad. Sci. U.S.A. 91:459-63 (1994); and Rosnet et al., Leukemia 10:238-48 (1996). Flt3 is also highly expressed in hematologic malignancies including acute myelogenous leukemia, B-precursor cell acute lymphoblastic leukemias, myelodysplastic leukemias, T-cell acute lymphoblastic leukemias, and chronic myelongenous leukemias. Stimlation of Flt3 receptor by its ligand activates signal transduction pathways that include STAT5, phosphotidylinositol 3′-kinase, PLCγ, MAPK, SHC, SHP2, and SHIP. See, e.g., Gilliand et al., Curr. Opin. Hematol. 9: 274-81 (2002). Both membrane-bound and soluble FL bind and activate the Flt3 receptor.

In one embodiment, the Flt3 ligand, or a biologically active fragment thereof, stimulates the proliferation of hematopoietic stem or progenitor cells. In a specific embodiment, the Flt3 ligand, or a biologically active fragment thereof, can stimulate the proliferation of cells selected from the group consisting of myeloid precursor cells, monocytic cells, macrophages, B-cells, dendritic cells (DCs) and natural killer (NK) cells. Flt3 ligand is expressed primarily by hematopoietic cells and other cells in the bone marrow environment, including fibroblasts, and B, T, and myeloid cell precursors. Flt3 ligand is a growth factor for CD34+ progenitor cells, and stimulates both growth and differentiation of dendritic cells and NK cells. For example, one study suggested that Flt3 mediated significant anti-tumor activity through the activation of NK cells. Péron et al., J. Immunol. 161:6164-70 (1998).

Flt3 ligand also promotes the maturation of DCs, rendering DCs more efficient as antigen presenting cells for tumor antigens. See, e.g., Fong et al., Gene Ther. 9(17):1127-38 (2002). More importantly, the mature DCs are released from bone marrow to peripheral tissues when induced by Flt3 ligand, thereby increasing the number of antigen presenting cells available to stimulate an immune response. However, the efficient induction of proliferation by Flt3 ligand typically requires the presence of other hematopoietic growth factors and interleukins.

Any biologically fragment of FL can be used in the present compositions and methods. As used herein, the term “biologically active” refers to a derivative or fragment of FL that still substantially retains its function as an stimulator of Flt3. Typically, Flt3 ligand binds Flt3 on the cell, stimulates one or more signal transduction pathways, and results in a cellular response, e.g., proliferation. Normally, the derivative or fragment retains at least 50% of its Flt3 stimulating activity. Preferably, the derivative or fragment retains at least 60%, 70%, 80%, 90%, 95%, 99% and 100% of its Flt3 stimulating activity. Flt3 stimulating activity can be determined by any suitable method, including but not limited to, determining the activation of signaling molecules, e.g., STAT5, PLCγ, or assessing proliferative activity in vitro in a Flt3 dependent cell line. For example, the BAF/BO3 cell line lacks the flt3 receptor and is IL-3 dependent. However, the transfection of BAF/BO3 cell line with Flt3 renders it responsive to Flt3 ligand-induced proliferation. See Hatakeyama, et al., Cell 59:837-45 (1989).

In one embodiment, the Flt3 ligand, or biologically active fragment thereof, in the chimeric protein has the amino acid sequence of SEQ ID NO:2. In one embodiment, the Flt3 ligand, binds to an antibody that specifically binds to an amino acid sequence set forth in SEQ ID NO:2, and the Flt3 ligand substantially retains its biological activity. Any suitable Flt3 ligand-specific antibody can be employed. In another embodiment, the Flt3 ligand comprises an amino acid sequence that is at least 80% identical to amino acids 28 to 128 of SEQ ID NO:2. In yet another embodiment, the Flt3 ligand comprises an amino acid sequence selected from the group consisting of amino acid residues 28-160 of SEQ ID NO:2, and amino acid residues 28-182 of SEQ ID NO:2. In a specific embodiment, the Flt3 ligand comprises amino acids 28 to 128 of SEQ ID NO:2. In another embodiment, the Flt3 ligand comprises at least 100 amino acid residues and the Flt3 ligand has at least 40% identity to the amino acid sequence set forth in SEQ ID NO:2, in which the percentage identity is determined over an amino acid sequence of identical size to the amino acid sequence set forth in SEQ ID NO:2, and the Flt3 ligand substantially retains its biological activity.

Any tumoricidal agent, or biologically active fragment thereof, can be used in the methods and compositions provided herein. As used herein, the term “tumoricidal agent” refers to an agent that causes the death of the tumor cell. The tumoricidal agent is preferably proteinaceous or peptidyl. The cell death can be apoptotic, necrotic, and the like. In one embodiment, the cell death results from apoptosis. Apoptosis can be induced directly through a ligand that induces an apoptotic signaling pathway, e.g, Fas ligand, or indirectly through, e.g., growth factor deprivation. As used herein, the term “apoptosis” refers to the programmed cell death of the tumor cell that ultimately results in a condensation of chromatin and fragmentation of the DNA. Any suitable method can be used to assess apoptosis including, but not limited to flow cytometric analysis, e.g., Terminal deoxynucleotidyl Transferase Biotin-dUTP Nick End Labeling (TUNEL) analysis, agarose gel analysis, and caspase 3 activation. In another embodiment, the tumoricidal agent of the chimeric protein is a naturally occurring anti-tumor agent. Such agents include ligands of receptors that induce stasis or cell death in tumor cells. Exemplary naturally occurring molecules, e.g., ligands, inducing apoptosis include TNF-α, Fas (CD95) ligand, TNF-related apoptosis-inducing ligand (TRAIL), lymphotoxin (LT), TWEAK, and other members of the TNF ligand superfamily. In one embodiment, the tumoricidal agent is selected from the group consisting of Fas ligand, TNF, TRAIL, or a biologically active extracellular domain thereof. See, e.g., In another embodiment, the A biologically active fragment of the tumoricidal agent retains at least 50% of its apoptotic activity. Preferably, the derivative or fragment retains at least 60%, 70%, 80%, 90%, 95%, 99% and 100% of its apoptotic activity.

In another embodiment, the tumoricidal agent of the chimeric protein is an antibody that inhibits the proliferation of a tumor and, in some cases, induces apoptosis. Exemplary targets of such antibodies include growth factor receptors. For example, the epidermal growth factor receptor (EGFR) subfamily is composed by EGFR, HER2 (a.k.a. HER-2), HER3 (a.k.a. HER-3) and HER4 (a.k.a. HER-4), all of which are transmembrane proteins with tyrosine kinase activities. These proteins are expressed at high levels in numerous malignancies, including prostate cancer, colon cancer, breast cancer, pancreas cancer, kidney cancer, ovary cancer, and lung cancer. Specific anti-EGFR or anti-HER2 mAbs can block the binding of EGFR or HER2 to their ligands and sequentially block the proliferation signaling pathways of tumor to inhibit tumor growth and induce tumor cell apoptosis directly or indirectly. See e.g., Clin. Cancer Res. 8:1720-30 (2002); Brodowicz et al. Br. J. Cancer 85:1764-70 (2001); Crombet-Ramos et al., Int. J. Cancer 101: 567-75 (2002); Herbst et al., Expert Opin. Biol. Ther. 1:719-32 (2001).

In yet another embodiment, the tumoricidal agent of the chimeric protein is an antibody that binds a tumor-specific or tumor-associated antigen that induces apoptosis. For example, p230 is a protein that specifically expressed in human liver cancer, breast cancer, and melanoma cells. Its name derives from a 230 KD band observed during Western blotting using mAb SM5-1. See U.S. patent application Ser. No. 09/915,746. P230 is suitable for cancer therapy. Apoptosis can be induced by combining P230 with its ligands or an antibody. Some anti-SM5-1 antibodies are described in Example 3. In a specific embodiment, the antibody is the SM5-1 antibody disclosed in copending application Ser. No. (U.S. Ser. No. 10/723,003; Attorney Docket No. 54906-2000100; title: ANTIBODIES SPECIFIC FOR CANCER ASSOCIATED ANTIGEN SM5-1 AND USES THEREOF), filed Nov. 26, 2003, which is incorporated in its entirety by reference. The humanized anti-SM5-1 antibody (hSM) described herein is designated as ReSM5-1 in that copending application.

In one embodiment, the tumoricidal agent is an antibody or a biologically active fragment thereof. As used herein, the term “antibody” refers to an intact antibody, a Fab fragment, a Fab′ fragment, a F(ab′)₂ fragment, a Fv fragment, a diabody, a single-chain antibody and a multi-specific antibody formed from antibody fragments, where the molecule retains substantially all of its desired biologic activity. Antibody includes any fragment that retains substantially all if its binding specificity for the target antigen. The antibodies useful in the present methods and compositions can be generated in cell culture, in phage, or in various animals, including but not limited to cows, rabbits, goats, mice, rats, hamsters, guinea pigs, sheep, dogs, cats, monkeys, chimpanzees, apes. Therefore, the antibody useful in the present methods is a mammalian antibody.

Phage techniques can be used to isolate an initial antibody or to generate variants with altered specificity or avidity characteristics. Such techniques are routine and well known in the art. In one embodiment, the antibody is produced by recombinant means known in the art. For example, a recombinant antibody can be produced by transfecting a host cell with a vector comprising a DNA sequence encoding the antibody. One or more vectors can be used to transfect the DNA sequence expressing at least one V_{L} and one V_{H} region in the host cell. Exemplary descriptions of recombinant means of antibody generation and production include Delves, ANTIBODY PRODUCTION: ESSENTIAL TECHNIQUES (Wiley, 1997); Shephard, et al., MONOCLONAL ANTIBODIES (Oxford University Press, 2000); and Goding, MONOCLONAL ANTIBODIES: PRINCIPLES AND PRACTICE (Academic Press, 1993).

The antibody useful in the present methods can be modified by recombinant means to increase greater efficacy of the antibody in mediating the desired function. It is also contemplated that antibodies can be modified by substitutions using recombinant means. Typically, the substitutions will be conservative substitutions. For example, at least one amino acid in the constant region of the antibody can be replaced with a different residue. See, e.g., U.S. Pat. No. 5,624,821, U.S. Pat. No. 6,194,551, Application No. WO 9958572; and Angal et al., Mol. Immunol. 30: 105-08 (1993). The modification in amino acids includes deletions, additions, substitutions of amino acids. In some cases, such changes are made to reduce undesired activities, e.g., complement-dependent cytotoxicity.

The antibody can be a humanized antibody. As used herein, the term “humanized antibody” refers to an antibody where the amino acid sequence in the non-antigen binding regions are altered so that the antibody more closely resembles a human antibody while still retaining it original antigen specificity. Typically, the variable regions are of one species, e.g., mouse, and the constant regions are human in origin. The antibody can be a chimeric antibody. As used herein, the term “chimeric antibody” refers to an antibody where the amino acid sequences are altered so that the antibody contains sequences from more than one mammal while still retaining it original antigen specificity. As used herein, the term “single-chain variable fragment (ScFv or sFv)” refers to a genetically engineered antibody that consists of the variable heavy chain (V_{H}) and variable light chain (V_{L}) of an immunoglobulin joined together by a flexible peptide linker.

Preferably, the antibody of the present methods and compositions is monoclonal. As used herein, the term “monoclonal antibody” refers to a singular antibody produced by a single B cell or hybridoma.

The antibody can be a human antibody. As used herein, the term “human antibody” refers to an antibody in which essentially the entire sequences of the light chain and heavy chain sequences, including the complementary determining regions (CDRs), are from human genes. In one embodiment, human monoclonal antibodies are prepared by the trioma technique, the human B-cell technique (see, e.g., Kozbor, et al., Immunol. Today 4; 72 (1983), EBV transformation technique (see, e.g., Cole et al. MONOCLONAL ANTIBODIES AND CANCER THERAPY 77-96 (1985)), or using phage display (see, e.g., Marks et al., J. Mol. Biol. 222:581 (1991)). In a specific embodiment, the human antibody is generated in a transgenic mouse. Techniques for making such partially to fully human antibodies are known in the art and any such techniques can be used. According to one particularly preferred embodiment, fully human antibody sequences are made in a transgenic mouse engineered to express human heavy and light chain antibody genes. An exemplary description of preparing transgenic mice that produce human antibodies found in Application No. WO 02/43478. B cells from transgenic mice that produce the desired antibody can then be fused to make hybridoma cell lines for continuous production of the monoclonal antibody. See, e.g., U.S. Pat. Nos. 5,569,825; 5,625,126; 5,633,425; 5,661,016; and 5,545,806; and Jakobovits, Adv. Drug Del. Rev. 31: 33-42 (1998); Green, et al., J. Exp. Med. 188: 483-495 (1998).

In one embodiment, the antibody provided herein inhibits the proliferation of the targeted tumor cells. An antibody is inhibitory for proliferation if it inhibits the proliferation of cells relative to the proliferation of cells in the absence of the antibody or in the presence of a non-binding antibody. Proliferation may be quantified using any suitable methods. Typically, the proliferation is determined by assessing the incorporation of radioactive-labeled nucleotides into DNA (e.g., ³H-thymidine) in vitro. In one embodiment, proliferation is determined by ATP luminescence, e.g., CellTiter-Glo™ Luminescent Cell Viability Assay (Promega). Therefore, the antibody can be specific for or target any molecule that modulates cell viability or cell growth.

In one embodiment, the antibody is selected from the group consisting of an anti-p230 antibody, an anti-CD20 antibody, an anti-Her2 antibody, an anti-Her3 antibody, an anti-Her4 antibody, an anti-EGFR antibody or a biologically active fragment thereof. Exemplary embodiments of these antibodies include those disclosed in the Example section infra as well as in, e.g., U.S. Pat. Nos. 5,677,171; 6,399,061; 6,458,356; 6,455,043; and 5,705,157.

The chimeric protein comprising Flt3 ligand, or a biologically active fragment thereof, and a tumoricidal agent can be linked by any suitable linkage. For example, the Flt3 ligand and tumoricidal agent can be linked by a peptidyl linker, a cleavable linker, and the like. In a specific embodiment, the linking peptide is (Gly₄Ser)₃ or the hinge domain from an immunoglobulin heavy chain.

The chimeric protein of the compositions and methods herein can comprise the Flt3 ligand and tumoricidal agent linked in any order. In one embodiment, the Flt3 ligand is located at the N-terminus of the chimeric protein. In another embodiment, the Flt3 ligand is located at the C-terminus of the chimeric protein.

The chimeric protein can further comprise, at its C-terminus, a peptidyl fragment comprising a peptidyl tag. Any suitable tag can be used. For example, the tag can be FLAG, HA, HA1, c-Myc, 6-His, AUI, EE, T7, 4A6, ε, B, gE and Ty1 tag (See Table 2). Such tags are useful in purification protocols for the chimeric protein.

TABLE 2
Exemplary epitope tag systems

| | | | | |
|---|---|---|---|---|
| Epitope | Peptide | SEQ ID | Antibody | Reference |

| | | | | |
|---|---|---|---|---|
| FLAG | AspTyrLysAspAspAspLys | 11 | 4E11 | Prickett¹ |
| HA | TyrProTyrAspValPRoAspTyrAla | 12 | 12Ca5 | Xie² |
| HA 1 | CysGlnAspLeuProGlyAsnAspAsnSerThr | 13 | mouse | Nagelkerken³ |
| | | | MAb | |
| c-Myc | GluGlnLysLeuIleSerGluGluAspLeu | 14 | 9E10 | Xie² |
| 6-His | HisHisHisHisHisHis | 15 | BAbCO* | |
| AUl | AspThrTyrArgTyrIle | 16 | BAbCO | |
| EE | GluTyrMetProMetGlu | 17 | anti-EE | Tolbert⁴ |
| T7 | AlaSerMetThrGlyGlyGlnGlnMetGlyArg | 18 | Invitrogen | Chen⁵ |
| | | | | Tseng⁶ |
| 4A6 | SerPheProGlnPheLysProGlnGlulle | 19 | 4A6 | Rudiger⁷ |
| ε | LysGlyPheSerTyrPheGlyGluAspLeuMetP | 20 | anti-PKCε | Olah⁸ |
| | ro | | | |
| B | GlnTyrProAlaLeuThr | 21 | D11, F10 | Wang⁹ |
| gE | GlnArgGlnTyrGlyAspValPheLysGlyAsp | 22 | 3B3 | Grose¹⁰ |
| Ty1 | GluValHisThrAsnGlnAspProLeuAsp | 23 | BB2, TYG5 | Bastin¹¹ |

¹Prickett, et al., BioTechniques, 7(6):580-584 (1989)
²Xie, et al., Endocrinology, 139(11):4563-4567 (1998)
³Nagelkerke, et al., Electrophoresis, 18:2694-2698 (1997)
⁴Tolbert and Lameh, J. Neurochem., 70:113-119 (1998)
⁵Chen and Katz, BioTechniques, 25(1):22-24 (1998)
⁶Tseng and Verma, Gene, 169:287-288 (1996)
⁷Rudiger, et al., BioTechniques, 23(1):96-97 (1997)
⁸Olah, et al., Biochem., 221:94-102 (1994)
⁹Wang, et al., Gene, 169(1):53-58 (1996)
¹⁰Grose, U.S. Pat. No. 5,710,248
¹¹Bastin, et al., Mol. Biochem. Parasitology, 77:235-239 (1996) Invitrogen, Sigma, Santa Cruz Biotech

In one embodiment, the chimeric protein comprises the amino acid sequence set forth in SEQ ID NO:24, SEQ ID NO:26, SEQ ID NO:28, SEQ ID NO:30, SEQ ID NO:32, SEQ ID NO:34, SEQ ID NO:44, SEQ ID NO:46, SEQ ID NO:48, SEQ ID NO:58, SEQ ID NO:60, SEQ ID NO:62, SEQ ID NO:66 or SEQ ID NO:68.

In another aspect, the present invention is directed to an isolated nucleic acid, or a complementary strand thereof, encoding a chimeric protein, which chimeric protein comprises a Flt3 ligand, or a biologically active fragment thereof, and a proteinaceous or peptidyl tumoricidal agent. In one embodiment, the chimeric protein is encoded by an isolated nucleic acid comprising the nucleotide sequence set forth in NO:23, SEQ ID NO:25, SEQ ID NO:27, SEQ ID NO:29, SEQ ID NO:31, SEQ ID NO:33, SEQ ID NO:43, SEQ ID NO:45, SEQ ID NO:47, SEQ ID NO:57, SEQ ID NO:59, SEQ ID NO:61, SEQ ID NO:65 or SEQ ID NO:67. A vector containing the isolated nucleic acid encoding the chimeric protein is also contemplated. The vector can further comprise an enhancer (i.e. expression modulation sequence) operatively linked to the nucleic acid encoding the Flt3 ligand and the proteinaceous or peptidyl tumoricidal agent.

Any suitable DNA construct encoding Flt3 ligand or a biologically active fragment thereof could be used in the present invention. Such constructs include, but are not limited to the nucleic acid sequences at Genbank accession number U03858 and ATCC accession number ATCC 69382. Further contemplated for use in the present invention are the DNA sequences and resultant proteins described in U.S. Pat. No. 5,843,423; and U.S. patent application Ser. Nos: 200030113341 and 20030148516.

Any suitable DNA construct encoding the tumoricidal agent, or a biologically active fragment thereof, may be employed in the compositions and methods herein. Exemplary sequences include those disclosed in the Example section infra.

Any suitable vector may be employed. Exemplary cloning and expression vectors for use with bacterial, fungal, yeast, and mammalian cellular host are described, e.g., in Pouwels et al., CLONING VECTORS: A LABORATORY MANUAL (Elsevier latest edition).

The expression vectors include a chimeric protein DNA sequence operably linked to suitable transcriptional or translational regulatory nucleotide sequences, such as those derived from a mammalian, microbial, viral, or insect gene. Examples of regulatory sequences include transcriptional promoters, operators, or enhancers, an mRNA ribosomal binding site, and appropriate sequences which control transcription and translation initiation and termination. Nucleotide sequences are “operably linked” when the regulatory sequence functionally relates to the chimeric protein DNA sequence. Thus, a promoter nucleotide sequence is operably linked to a chimeric protein-encoding DNA sequence if the promoter nucleotide sequence controls the transcription of the chimeric protein-encoding DNA sequence. The ability to replicate in the desired host cells, usually conferred by an origin of replication, and a selection gene by which transformants are identified, may additionally be incorporated into the expression vector.

In addition, sequences encoding appropriate signal peptides that are not naturally associated with the Flt-3 ligand or the tumoricidal agent can be incorporated into expression vectors. For example, a DNA sequence for a signal peptide (secretory leader) may be fused in-frame to the chimeric protein-encoding sequence so that the sequence is initially translated as a fusion protein comprising the signal peptide. A signal peptide that is functional in the intended host cells enhances extracellular secretion of the chimeric polypeptide. The signal peptide may be cleaved from the chimeric polypeptide upon secretion of the chimeric polypeptide from the cell.

Mammalian or insect host cell culture systems could also be employed to express recombinant chimeric polypeptides. Baculovirus systems for production of heterologous proteins in insect cells are reviewed by Luckow and Summers, Bio/Technology 6:47 (1988). Established cell lines of mammalian origin also may be employed. Examples of suitable mammalian host cell lines include the COS-7 line of monkey kidney cells (ATCC CRL 1651) (Gluzman et al., Cell 23:175, 1981), L cells, C127 cells, 3T3 cells (ATCC CCL 163), Chinese hamster ovary (CHO) cells, HeLa cells, and BHK (ATCC CRL 10) cell lines, and the CV-1/EBNA-1 cell line derived from the African green monkey kidney cell line CVI (ATCC CCL 70) as described by McMahan et al. (EMBO J. 10:2821, 1991), and the NSO cell line (Galfre et al., Methods Enzymol. 73:3-46 (1981)).

Transcriptional and translational control sequences for mammalian host cell expression vectors may be excised from viral genomes. Commonly used promoter sequences and enhancer sequences are derived from Polyoma virus, Adenovirus 2, Simian Virus 40 (SV40), and human cytomegalovirus. DNA sequences derived from the SV40 viral genome, for example, SV40 origin, early and late promoter, enhancer, splice, and polyadenylation sites may be used to provide other genetic elements for expression of a structural gene sequence in a mammalian host cell. Viral early and late promoters are particularly useful because both are easily obtained from a viral genome as a fragment which may also contain a viral origin of replication. See, e.g., Fiers et al., Nature 273:113 (1978). Smaller or larger SV40 fragments may also be used, provided the approximately 250 bp sequence extending from the Hind III site toward the Bgl I site located in the SV40 viral origin of replication site is included.

Exemplary expression vectors for use in mammalian host cells can be constructed as disclosed by Okayama and Berg, Mol. Cell. Biol. 3:280 (1983). A useful system for stable high level expression of mammalian cDNAs in C127 murine mammary epithelial cells can be constructed substantially as described by Cosman et al. (Mol. Immunol. 23:935, 1986). A useful high expression vector, PMLSV N1/N4, described by Cosman et al., Nature 312:768, 1984 has been deposited as ATCC 39890. Additional useful mammalian expression vectors are described in EP-A-0367566, and in U.S. patent application Ser. No. 07/701,415, incorporated by reference herein. The vectors may be derived from retroviruses. In place of the native signal sequence, a heterologous signal sequence may be added, such as the signal sequence for IL-7 described in U.S. Pat. No. 4,965,195; the signal sequence for IL-2 receptor described in Cosman et al., Nature 312:768 (1984); the IL-4 signal peptide described in EP 367,566; the type I IL-1 receptor signal peptide described in U.S. Pat. No. 4,968,607; and the type II IL-1 receptor signal peptide described in EP 460,846.

A method of producing a chimeric protein is also contemplated, which method comprising growing a recombinant cell containing the nucleic acid encoding a chimeric protein, which chimeric protein comprises a Flt3 ligand, or a biologically active fragment therof, and a tumoricidal agent, such that the encoded chimeric protein is expressed by the cell, and recovering the expressed chimeric protein. In one embodiment, the method further comprises isolating and/or purifying the recovered chimeric protein. The product of the method is further contemplated. The chimeric protein can be purified to substantial homogeneity, as indicated by a single protein band upon analysis by SDS-polyacrylamide gel electrophoresis (SDS-PAGE). For example, when expression systems that secrete the recombinant protein are employed, the culture medium first may be concentrated using a commercially available protein concentration filter, for example, an Amicon or Millipore Pellicon ultrafiltration unit. Following the concentration step, the concentrate can be applied to a purification matrix such as a gel filtration medium. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Sulfopropyl groups are preferred. Finally, one or more reversed-phase high performance liquid chromatography (RP-HPLC) steps employing hydrophobic RP-HPLC media, (e.g., silica gel having pendant methyl or other aliphatic groups) can be employed to further purify the chimeric protein. Some or all of the foregoing purification steps, in various combinations, are well known and can be employed to provide a substantially homogeneous recombinant protein.

It is possible to utilize an affinity column comprising the ligand binding domain of flt3 receptors to affinity-purify expressed the chimeric polypeptides. The chimeric polypeptides can be removed from an affinity column using conventional techniques, e.g., in a high salt elution buffer and then dialyzed into a lower salt buffer for use or by changing pH or other components depending on the affinity matrix utilized. Alternatively, the affinity column may comprise an antibody that binds FL.

Transformed yeast host cells can also be employed to express the chimeric protein as a secreted polypeptide in order to simplify purification. Secreted recombinant polypeptide from a yeast host cell fermentation can be purified by methods analogous to those disclosed by Urdal et al. (J. Chromatog. 296:171, 1984).

Recombinant cells comprising the nucleic acid are also provided. In one embodiment, the cell is an eukaryotic cell. In a specific embodiment, the cell is a CHO, COS, or NSO cell.

The chimeric proteins and the nucleic acids encoding the chimeric proteins can be prepared by any suitable methods, e.g., chemical synthesis, recombinant production or a combination thereof. See e.g., CURRENT PROTOCOLS IN MOLECULAR BIOLOGY, Ausubel, et al. eds., John Wiley & Sons, Inc. (2000) and Sambrook, et al., MOLECULAR CLONING: A LABORATORY MANUAL, Cold Spring Harbor Laboratory press, (1989). In an exemplary method, the nucleic acids encoding the chimeric proteins are prepared using recursive PCR techniques as disclosed in Prodromou et al., Protein Eng. 5(8):827-29 (1992).

Pharmaceutical compositions comprising the chimeric protein comprising Flt3 ligand, or a biologically active fragment thereof, and a proteinaceous or peptidyl tumoricidal agent and a pharmaceutically acceptable carrier or excipient are contemplated. Pharmaceutical compositions for use in accordance with the present methods thus may be formulated in a conventional manner using one or more physiologically acceptable carriers comprising excipients and auxiliaries that facilitate processing of the active compounds into preparations that can be used pharmaceutically. These pharmaceutical compositions may be manufactured in a manner that is itself known, e.g., by means of conventional mixing, dissolving, granulating, dragee-making, levigating, emulsifying, encapsulating, entrapping or lyophilizing processes. Proper formulation is dependent upon the route of administration chosen. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of protein of the present invention, and preferably from about 1 to 50% protein of the present invention.

In another aspect, provided herein is a combination, which combination comprises: a) an effective amount of a chimeric protein comprising a Flt3 ligand, or a biologically active fragment thereof, and a proteinaceous or peptidyl tumoricidal agent; and b) an effective amount of an anti-neoplastic agent. The anti-neoplastic agent of the combination is preferably other than the proteinaceous or peptidyl tumoricidal agent. In one embodiment, the anti-neoplastic agent is an agent that inhibits the growth of melanoma, breast cancer or hepatocellular carcinoma. Growth inhibition can occur through the induction of stasis or cell death in the tumor cell(s). Exemplary anti-neoplastic agents include cytokines, ligands, antibodies, radionuclides, and chemotherapeutic agents. Such agents include interleukin 2 (IL-2), interferon (IFN) TNF; photosensitizers, including aluminum (III) phthalocyanine tetrasulfonate, hematoporphyrin, and phthalocyanine; radionuclides, such as iodine-131 (¹³¹I), yttrium-90 (⁹⁰Y), bismuth-212 (²¹²Bi), bismuth-213 (²¹³Bi), technetium-99m (^{99m}Tc), rhenium-186 (¹⁸⁶Re), and rhenium-188 (¹⁸⁸Re); chemotherapeutics, such as doxorubicin, adriamycin, daunorubicin, methotrexate, daunomycin, neocarzinostatin, and carboplatin; bacterial, plant, and other toxins, such as diphtheria toxin, pseudomonas exotoxin A, staphylococcal enterotoxin A, abrin-A toxin, ricin A (deglycosylated ricin A and native ricin A), TGF-α toxin, Cytotoxin from chinese cobra (naja naja atra), and gelonin (a plant toxin); ribosome inactivating proteins from plants, bacteria and fungi, such as restrictocin (a ribosome inactivating protein produced by Aspergillus restrictus), saporin (a ribosome inactivating protein from Saponaria officinalis), and RNase; tyrosine kinase inhibitors; ly207702 (a difluorinated purine nucleoside); liposomes containing antitumor agents (e.g., antisense oligonucleotides, plasmids encoding toxins, methotrexate, etc.); and other antibodies or antibody fragments, such as F(ab).

In one aspect, kits are provided for carrying out the methods disclosed herein. Such kits comprise in one or more containers effective amounts of the chimeric protein comprising a Flt3 ligand, or a biologically active fragment thereof, and a proteinaceous or peptidyl tumoricidal agent in a pharmaceutically acceptable form, and an instructions means for administering the chimeric protein is contemplated. In one embodiment, the kit further comprises an effective amount of an anti-neoplastic agent as disclosed above. Preferred pharmaceutical forms would be in combination with sterile saline, dextrose solution, or buffered solution, or other pharmaceutically acceptable sterile fluid. Alternatively, the composition may be lyophilized or dessicated; in this instance, the kit optionally further comprises in a container a pharmaceutically acceptable solution, preferably sterile, to reconstitute the complex to form a solution for injection purposes. Exemplary pharmaceutically acceptable solutions are saline and dextrose solution. In another embodiment, a kit of the invention further comprises a needle or syringe, preferably packaged in sterile form, for injecting the composition, and/or a packaged alcohol pad. Instructions are optionally included for administration of composition by a physician or by the patient.

As used herein, the term “therapeutically effective amount” or “effective amount” refers to an amount of a chimeric protein (or expression vector encoding the chimeric protein) that when administered alone or in combination with an additional therapeutic agent to a cell, tissue, or subject is effective to prevent or ameliorate the tumor or tumor-associated disease condition or the progression of the tumor growth. A therapeutically effective dose further refers to that amount of the compound sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously.

C. Methods Employing the Chimeric Protein Comprising Flt3 Ligand and a Tumoricidal Agent

In another aspect, provided herein is a method for inducing caspase-3 mediated apoptosis in a cell, which method comprises contacting the cell with an effective amount of a chimeric protein comprising a Flt3 ligand, or a biologically active fragment thereof, and a proteinaceous or peptidyl tumoricidal agent. In one embodiment, the cell is a mammalian cell. In a specific embodiment, the cell is a mammalian neoplastic cell. In one embodiment, the cell is contained in a mammal. In another embodiment, the cell expresses a target for the tumoricidal agent.

Caspase activation plays a critical role in the apoptotic changes in a cell. See e.g., Budihardjo et al., Ann. Rev. Cell Dev. Biol 15: 269-90 (1999). Caspases are a family of cysteine proteases with a high degree of specificity, i.e., an absolute requirement for cleavage after an aspartic acid and a recognition sequence of at least four amino acids N-terminal to the cleavage site. See e.g., Grutter, Curr. Op. Struct. Biol. 10: 649-55 (2000). Caspase 3, also known as CPP32, YAMA, and apopain, has a specificity for WEHD cleavage sites. It is a downstream or executioner caspase, acting to cleave various substrates such as lamins, PARP, DFF, and others. Existing intracellularly as an inactive zymogen, caspase 3 is activated following cleavage by caspase 9 and Apaf-1, upstream capases, activated following an extracellular apoptotic stimuli resulting from ligands such as Fas ligand, TNF, or TRAIL binding to their appropriate receptor. Caspase activation can be readily determined using well known methods in the art. Exemplary methods can be found in, e.g., APOPTOSIS: A PRACTICAL APPROACH (Studzinski, ed. 1999).

Caspase 3 is a member of a family of cysteine proteases critical in apoptosis or programmed cell death. See, e.g., Grütter, Curr. Opin. Structural Biol. 10:649-55 (2000); Budihardjo et al., Annu. Rev. Cell. Dev. Biol. 15:269-90 (1999). Caspase 3 exists as a proenzyme within a cell and is activated by proteolysis, typically by an “initiator” caspase, e.g., caspase-8, -9, or 10. The active caspase-3 then cleaves other proteins, primarily those involved in DNA repair processes or structural components of the cytoskeleton or nuclear scaffold, at sites that contain the recognition sequence DEVD after an aspartic acid. The detection of caspase 3 activation is routine and well known in the art. See, e.g., U.S. Pat. Nos. 6,342,611; 6,391,575; 6,335,429; and U.S. application Ser. No. 20030186214. Thus, any suitable method of detecting caspase 3 activation may be employed herein.

Provided herein are methods employing the chimeric protein comprising a Flt3 ligand, or a biologically active fragment thereof, and a proteinaceous or peptidyl tumoricidal agent treat a neoplasm (or cancer) in a mammal, which method comprises administering to a mammal to which such treatment is needed or desirable, an effective amount of the chimeric protein as disclosed in Section B supra. In one embodiment, the neoplasm is melanoma, breast cancer or hepatocellular carcinoma.

The expression vectors encoding the Flt3 ligand chimeric proteins of the invention also may be administered to an individual with cancer to obtain expression of the therapeutic chimeric protein in vivo. Suitable expression vectors for delivery a gene sought to be expressed in vivo are well known in the art and include, for example, adenoviral vectors, adeno-associated vial vectors, and the like.

In yet another aspect, provided herein is a method for producing a tumor-specific lymphocyte, which method comprises administering to a mammal an effective amount of a chimeric protein comprising a Flt3 ligand, or a biologically active fragment thereof, and a proteinaceous or peptidyl tumoricidal agent to generate a tumor-specific lymphocyte, and recovering the generated tumor-specific lymphocyte from the mammal.

A method of administering an effective amount of the combination of the chimeric protein disclosed in Section B and an anti-neoplastic agent disclosed in Section B to treat neoplasms in a mammal, wherein such treatment is needed or desirable is also contemplated.

Any subject can be treated with the methods and compositions provided herein. Such a subject is a mammal, preferably a human. In one specific embodiment, the subject has cancer. Veterinary uses of the disclosed methods and compositions are also contemplated.

The subject with a neoplasm or cancer includes adenocarcinoma, leukemia, lymphoma, melanoma, sarcoma, or tetratocarcinoma. The tumor can be a cancer of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus. Such tumors include, but are not limited to: neoplasms of the central nervous system: glioblastomamultiforme, astrocytoma, oligodendroglial tumors, ependymal and choroids plexus tumors, pineal tumors, neuronal tumors, medulloblastoma, schwannoma, meningioma, meningeal sarcoma: neoplasma of the eye: basal cell carcinoma, squamous cell carcinoma, melanoma, rhabdomyosarcoma, retinoblastoma; neoplasma of the enbdocrine glands: pituitary neoplasms, neoplasms of the thyroid, neoplasms of the adrenal cortex, neoplasms of the neuroendocrine system, neoplasms of the gastroenteropancreatic endocrine system, neoplasms of the gonads; neoplasms of the head and neck: head and neck cancer, oral cavity, pharynx, larynx, odontogenic tumors: neoplasms of the thorax: large cell lung carcinoma, small cell lung carcinoma, non-small cell lung carcinoma, neoplasms of the thorax, malignant mesothelioma, thymomas, primary germ cell tumors of the thorax; neoplasms of the alimentary canal: neoplasms of the esophagus, neoplasms of the stomach, neoplasms of the liver, neoplasms of the gallbladder, neoplasms of the exocrine pancreas, neoplasms of the small intestine, vermiform appendix and peritoneum, adenocarcinoma of the colon and rectum, neoplasms of the anus; neoplasms of the genitourinary tract: renal cell carcinoma, neoplasms of the renal pelvis and ureter, neoplasms of the bladder, neoplasms of the urethra, neoplasms of the prostate, neoplasms of the penis, neoplasms of the testis; neoplasms of the female reproductive organs: neoplasms of the vulva and vagina, neoplasms of the cervix, adenocarcinoma of the uterine corpus, ovarian cancer, gynecologic sarcomas; neoplasms of the breast; neoplasms of the skin: basal cell carcinoma, squamous carcinoma, dermatofibrosarcoma, Merkel cell tumor; malignant melanoma; neoplasms of the bone and soft tissue: osteogenic sarcoma, malignant fibrous histiocytoma, chrondrosarcoma, Ewing's sarcoma, primitive neuroectodermal tumor, angiosarcoma; neoplasms of the hematipoeitic system: myelodysplastic syndromes, acute myeloid leukemia, chronic myeloid leukemia, acute lymphocytic leukemia, HTLV-1, and T-cell leukemia/lymphoma, chronic lymphocytic leukemia, hairy cell leukemia, Hodgkin's disease, non-Hodgkin's lymphomas, mast cell leukemia; neoplasms of children: acute lymphoblastic leukemia, acute myelocytic leukemias, neuroblastoma, bone tumors, rhabdomyosarcoma, lymphomas, renal and liver tumors.

As used herein, “inhibit” or “treat” or “treatment” includes a postponement of development of the symptoms associated with uncontrolled tumor cell growth and/or a reduction in the severity of such symptoms that will or are expected to develop. The terms further include ameliorating existing uncontrolled or unwanted or tumor growth-related symptoms, preventing additional symptoms, and ameliorating or preventing the underlying metabolic causes of symptoms. Thus, the terms denote that a beneficial result has been conferred on a mammal with a malignancy, or with the potential to develop such a disease or symptom.

In practicing the methods of treatment or use provided herein, a therapeutically effective amount of the chimeric protein provided herein is administered to a mammal having a condition to be treated. The chimeric protein may be administered in accordance with the methods herein either alone or in combination with other therapies such as treatments employing other immunopotentiating factors (e.g., cytokines), chemotherapeutic agents, anti-neoplastic agents, and the like. When co-administered with one or more biologically active agents, the chimeric protein provided herein may be administered either simultaneously with the biologically active agent(s), or sequentially. If administered sequentially, the attending physician will decide on the appropriate sequence of administering protein of the present invention in combination with the biologically active agent(s). Toxicity and therapeutic efficacy of such therapeutic regimens can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Chimeric proteins exhibiting high therapeutic indices are preferred. The data obtained from cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. See, e.g., Fingl et al., THE PHARMACOLOGICAL BASIS OF THERAPEUTICS 1 (latest edition). Dosage amount and interval may be adjusted individually to provide plasma levels of the active moiety sufficient to maintain the desired therapeutic effects, or minimal effective concentration (MEC). The MEC will vary for each compound but can be estimated from in vitro data; for example, the concentration necessary to achieve 50-90% inhibition of tumor proliferation using the assays described herein.

Any suitable route of administration may be used. The mode of administration is not particularly important. Dosage forms include tablets, troches, cachet, dispersions, suspensions, solutions, capsules, patches, and the like. See, e.g., REMINGTON'S PHARMACEUTICAL SCIENCES, Mack Publishing Co., Easton, Pa., latest edition.

In one embodiment, the mode of administration is an I.V. bolus. The prescribing physician will normally determine the dosage of the antibodies provided herein. It is to be expected that the dosage will vary according to the age, weight and response of the individual patient.

Techniques for formulation and administration of the proteins of the instant methods may be found in REMINGTON'S PHARMACEUTICAL SCIENCES, Mack Publishing Co., Easton, Pa., latest edition. It is contemplated that formulations and administration considerations for the chimeric protein provided herein will be similar to that of antibodies. Suitable routes of administration may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. Administration of the chimeric used in the pharmaceutical composition or to practice the method of the present invention can be carried out in a variety of conventional ways, such as oral ingestion, inhalation, topical application or cutaneous, subcutaneous, intraperitoneal, parenteral, intraarterial or intravenous injection. Intravenous administration to the patient is preferred.

Alternately, one may administer the chimeric protein in a local rather than systemic manner, for example, via injection of the antibody directly into a tumor, often in a depot or sustained release formulation. Furthermore, one may administer the chimeric protein in a targeted drug delivery system, for example, in a liposome coated with a tissue-specific antibody, targeting, e.g., a tumor. The liposomes will be targeted to and taken up selectively by the tumor tissue.

When a therapeutically effective amount of chimeric protein of the methods herein is administered by intravenous, cutaneous or subcutaneous injection, the protein provided herein will be in the form of a pyrogen-free, parenterally acceptable aqueous solution. The preparation of such parenterally acceptable protein solutions, having due regard to pH, isotonicity, stability, and the like, is within the skill in the art. A preferred pharmaceutical composition for intravenous, cutaneous, or subcutaneous injection should contain, in addition to protein of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art. For transmucosal administration, penetrants appropriate to the barrier to be permeated are used in the formulation. Such penetrants are generally known in the art.

For administration by inhalation, the chimeric proteins for use according to the present methods are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch. The compounds may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. Alternatively, the active ingredient may be in powder form for constitution with a suitable vehicle, e.g., sterile pyrogen-free water, before use.

The amount of chimeric antibody useful in the pharmaceutical composition provided herein will depend upon the nature and severity of the condition being treated, and on the nature of prior treatments that the patient has undergone. Ultimately, the attending physician will decide the amount of protein of the present invention with which to treat each individual patient. Initially, the attending physician will administer low doses of chimeric proteins of the present methods and observe the patient's response. Larger doses of chimeric proteins of the present invention may be administered until the optimal therapeutic effect is obtained for the patient, and at that point the dosage is not increased further. It is contemplated that the various pharmaceutical compositions used to practice the methods herein should contain about 0.01 μg to about 100 mg (preferably about 0.1 μg to about 10 mg, more preferably about 0.1 μg to about 1 mg) of chimeric proteins of the present invention per kg body weight. When administered, the therapeutic composition for use in this invention is, of course, in a pyrogen-free, physiologically acceptable form. Therapeutically useful agents other than a chimeric protein of the present methods that may also optionally be included in the composition as described above, may alternatively or additionally, be administered simultaneously or sequentially with the pharmaceutical composition in the methods of the invention. Exemplary agents to combine with the chimeric protein include anti-neoplastic agents as disclosed in Section C supra.

The chimeric protein provided herein can be administered alone or in combination with other therapeutic modalities. For example, the treatment method can further comprise a step of delivering ionizing radiation to the cells contacted with the chimeric protein. The ionizing radiation is delivered in a dose sufficient to induce a substantial degree of cell killing among the malignantly proliferating cells, as judged by assays measuring viable malignant cells. Preferably, the degree of cell killing induced is substantially greater than that induced by either the antibody alone or the ionizing radiation alone. Typical forms of ionizing radiation include beta rays, gamma rays, alpha particles, and X-rays. These can be delivered from an outside source, such as X-ray machine or a gamma camera, or delivered to the malignant tissue from radionuclides administered to the patient. Radionuclides can also be employed using methods well known in the art. The use of ionizing radiation in the treatment of malignancies is described, e.g., in S. Hellman, Principles of Radiation Therapy, in CANCER: PRINCIPLES & PRACTICE OF ONCOLOGY 248 (V. T. DeVita, Jr., et al., eds., 4th ed., 1993). Typically, range of dosages that can be used is between about 1 and 500 cGy (i.e., from about 1 to about 500 rads).

In one aspect, provided herein is a vaccine comprising an effective amount of a chimeric protein comprising a Flt3 ligand and a proteinaceous or peptidyl tumoricidal agent, and an immune response potentiator. The immune response potentiator is preferably other than Flt3 ligand.

In another aspect, provided herein is a method for eliciting an anti-neoplasm immune response in a mammal, which method comprises administering to a mammal to which such elicitation is needed or desirable, an effective amount of a vaccine comprising an effective amount of a chimeric protein comprising a Flt3 ligand and a proteinaceous or peptidyl tumoricidal agent, and an immune response potentiator. The immune response potentiator is preferably other than Flt3 ligand.

As used herein, the term “immune response potentiator” refers to any agent that enhances or prolongs the immune response to the target antigen, e.g., tumor antigen. The enhancement of the immune response can be additive or syngerstic. As used herein, the term “immune response” encompasses B cell-mediated, T-cell mediated, or a combination of both B- and T-cell mediated responses. Exemplary immune response potentiators include other cytokines, e.g., IL-12, IL-2, IFN-γ, adjuvants, immunostimulatory peptides, and the like. The immune response potentiators of the present composition and methods can be administered simultaneously or sequentially with the chimeric protein via the same administrative route or a different route.

Vaccination can be conducted by conventional methods. For example, the immunogen can be used in a suitable diluent such as saline or water, or complete or incomplete adjuvants. Further, the immunogen may or may not be bound to a carrier to make the protein immunogenic. Examples of such carrier molecules include but are not limited to bovine serum albumin (BSA), keyhole limpet hemocyanin (KLH), tetanus toxoid, and the like. The immunogen also may be coupled with lipoproteins or administered in liposomal form or with adjuvants. The immunogen can be administered by any route appropriate for antibody production such as intravenous, intraperitoneal, intramuscular, subcutaneous, and the like. The immunogen may be administered once or at periodic intervals until a significant titer of anti-tumor cell T cell response or anti-tumor cell antibody is produced. The presence of anti-tumor cell response may be assessed by measuring the frequency of precursor CTL (cytoxic T-lymphocytes) against the tumor antigen prior to and after immunization. See, e.g., Coulie, P. et al., Int. J. Cancer 50:289-97 (1992). The antibody may be detected in the serum using the immunoassay known in the art.

The administration of the vaccine of the present invention may be for either a prophylactic or therapeutic purpose. When provided prophylactically, the chimeric protein is provided in advance of any evidence or in advance of any symptom due to malignancy. The prophylactic administration of the chimeric protein serves to prevent or attenuate malignancy in a mammal, preferably a human. When provided therapeutically, the chimeric protein is provided at (or shortly after) the onset of the disease or at the onset of any symptom of the disease. The therapeutic administration of the chimeric protein serves to attenuate the disease.

Local administration to the afflicted site may be accomplished through means known in the art, including, but not limited to, topical application, injection, and implantation of a porous device containing cells recombinantly expressing the infusion, implantation of a porous device in which the chimeric protein alone or with immune response potentiators are contained.

The vaccine formulations may be evaluated first in animal models, initially rodents, and in nonhuman primates and finally in humans. The safety of the immunization procedures is determined by looking for the effect of immunization on the general health of the immunized animal (weight change, fever, appetite behavior etc.) and looking for pathological changes on autopsies. After initial testing in animals, cancer patients can be tested. Conventional methods would be used to evaluate the immune response of the patient to determine the efficiency of the vaccine. See, e.g., CURRENT PROTOCOLS IN IMMUNOLOGY (lastest edition). Examples of where T-lymphocytes can be isolated, include but are not limited to, peripheral blood cells lymphocytes (PBL), lymph nodes, or tumor infiltrating lymphocytes (TIL). Such lymphocytes can be isolated from the individual to be treated or from a donor by methods known in the art and cultured in vitro. See, e.g., Kawakami, Y. et al., J. Immunol. 142: 2453-61 (1989). Lymphocytes can be cultured in media using well known techniques in the art. Viability is assessed by trypan blue dye exclusion assay. Parameters that may be assessed to determine the efficacy of these sensitized T lymphocytes include, but are not limited to, production of immune cells in the mammal being treated or tumor regression. Conventional methods are used to assess these parameters. Such methods include cytotoxicity assays, mixed lymphocytes reactions, and cytokine production assays.

Any suitable tumor model can be used to provide a model for the testing of the chimeric proteins. The murine recipient of the tumor can be any suitable strain. The tumor can be syngeneic, allogeneic, or xenogenic to the tumor. The recipient can be immunocompetent or immunocompromised in one or more immune-related functions, included but not limited to nu/nu, scid, and beige mice. In one embodiment, the recipient is a transgenic mouse. In one specific embodiment, the mouse is a Balb/c or C57BL/6 mouse. Any suitable tumor source can be used for animal model experiments, including established cell lines, dissociated cells from fresh tumor samples, and short term polyclonal tumor cells. Exemplary tumor cell lines include Renca cells, B16 melanoma cells, Hepa1 cells, BT-474 cells, Raji cells, QYC cells, D2F2 cells, 4T1 cells, A20 cells. The dosage of chimeric protein ranges from 1 μg/mouse to 1 mg/mouse in at least one administration. The antibody can be administered by any suitable route. In one embodiment, the dose of antibody is 100 μg/mouse twice a week. In one specific embodiment, the tumor is injected subcutaneously at day 0, and the volume of the primary tumor is measured at designated time points by using calipers. Any suitable control protein can be used. In one example, the control antibody is a purified IgG, isotype control antibody which had been raised against a hapten, dinitrophenyl.

The broad scope of this invention is best understood with reference to the following examples, which are not intended to limit the inventions to the specific embodiments.

### D. EXAMPLES

### Example 1

### Human Flt3 Ligand Extracellular Region (hFLex) cDNA Synthesis

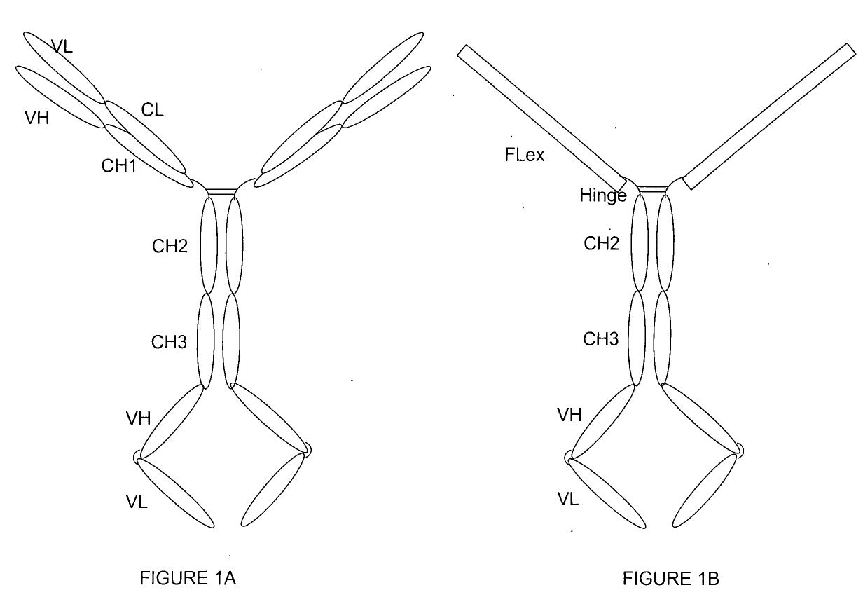

Purpose: Because the Flt3 ligand is a type I transmembrane protein whose extracellular region is at the N terminus, modification of the N terminus of FL may adversely affect its biological activities. Therefore, we employed a methodology used to construct a tetravalent biospecific antibody (see FIG. 1A). See Column et al., Nat Biotech 15:159-163 (1997). Typically, the tetravalent bispecific antibodies were constructed by fusing the DNA encoding a single chain antibody at the C terminus of an antibody with a different specificity. In order to obtain bifunctional fusion protein with high biological activities, we constructed a fusion protein with FLex at N the terminus and the antibody molecule at the C terminus (see FIG. 1B). First, the FLex gene was fused to the 5′end of a human IgG1 cDNA (hinge plus CH2 plus CH3) to generate the Flex-Ig fusion gene. Then the hFLex-Ig fusion gene was fused to the 5′ end of a single chain antibody gene to generate the Flex-Ig-scFv fusion gene.
hFLex cDNA synthesis: The cDNA sequence of the human FLt3 ligand gene, Genbank database with accession number U03858. Nucleotides 84 through 161 encoded the signal peptide of FLt3 ligand nucleotides 162 through 629 encoded the extracellular region of Flt3 ligand. Therefore, the size of gene encoding both signal peptide and extracellular region of Flt3 ligand was 546 bp.

The FLex gene segment was synthesized as described in Prodromou C et al., Protein Eng. 5 (8): 827-829. Briefly, the FLex cDNA was divided into 10 DNA fragments of approximately 75 bp. The fragments were designed using the following criteria: (1) each fragment overlaps with adjacent fragments in length of 20 bp; (2) the size of the last fragment may be shorter than 75 bp; and (3) the antisense chain is chosen for primer for the last fragment, and the sense chains are chosen for primers with regard to all the other fragments. The primers above then were commercially synthesized (Shengong Biotechnology Inc. (Shanghai, China)).

PCR was performed in the volume of 50 ul containing 85 nM of each primer, 1.5 mM MgCl₂, 200 mM dNTP, and 2.5 units of Pfu DNA polymerase. The PCR cycling protocol was: preincubation (94° C. for 5 minutes); 30 cycles of denaturation (94° C. for 1 minute), annealing (56° C. for 1 min), and extension at 72° C. The extension time varied according to the number of primers with the time calculated using the following equation: extension time (sec)=No. of primers×6 (sec)). The final extension was at 72° C. for 5 minutes.

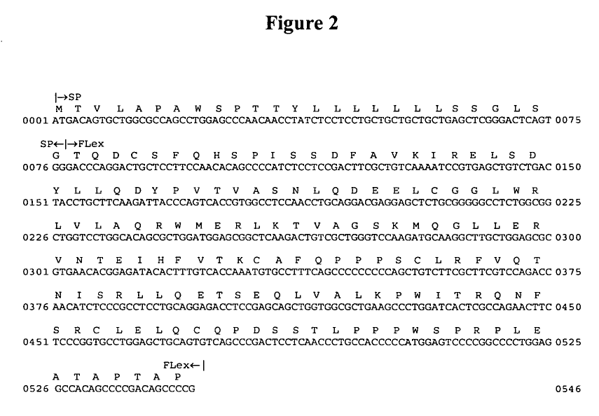

The PCR reaction products were separated on 1% agarose gel. The correct DNA fragment was gel-purified and cloned into pGEM-T vector (Promega), and its sequence was verified. See FIG. 2 (SEQ ID NOS: 1 and 2). The clone was denoted pGEM-T/hFlex.

### Example 2

### Cloning and Identification of the Constant Region of Human IgG1

The native human IgG1 cDNA of 1416 bp encodes 471 amino acids and a translation termination codon. The constant region of IgG1 was cloned by RT-PCR using the following protocol: Human peripheral blood mononuclear cells (PBMCs) were isolated from heparinized blood of healthy volunteers by Ficoll-Hypaque density gradient centrifugation. RNA was isolated from PBMCs with TRIzol Reagent (Gibco BRL). The cDNA of IgG1 Fc fragment was obtained by Onestep RT-PCR (Qiagen). The primers for RT-PCR were as follows: Fc sense, 5′-gca ctc gag ttt tac ccg gag aca ggg aga g-3′; Fc antisense, 5′-gag ccc aaa tct tgt gac aaa ac-3′. The RT-PCR products were separated on agarose gel. The correct DNA fragment was gel-purified and cloned into pGEM-T vector (Promega), and its sequence was verified. The clone was denoted pGEM-T/IgFc.

### Example 3

### Construction of SM5-1 Chimeric Antibody and Humanized Antibody

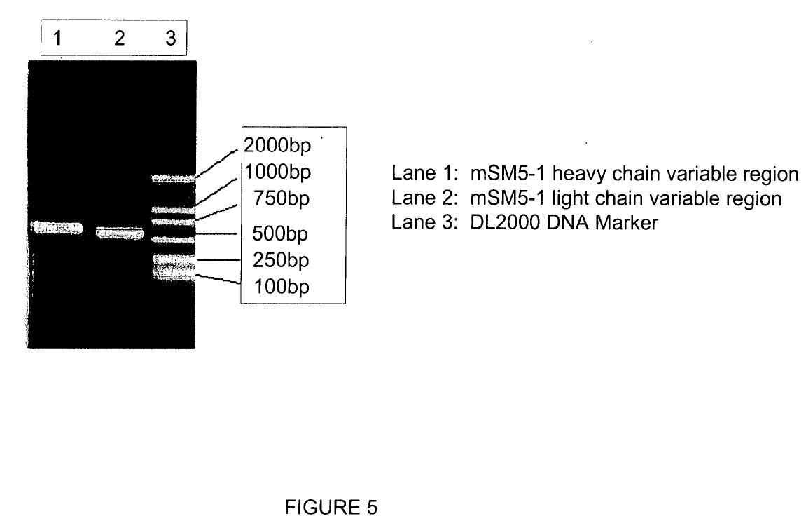

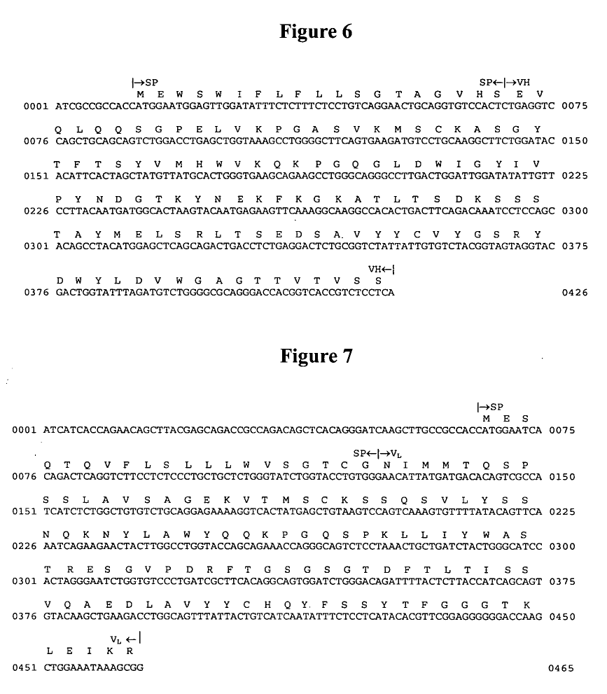

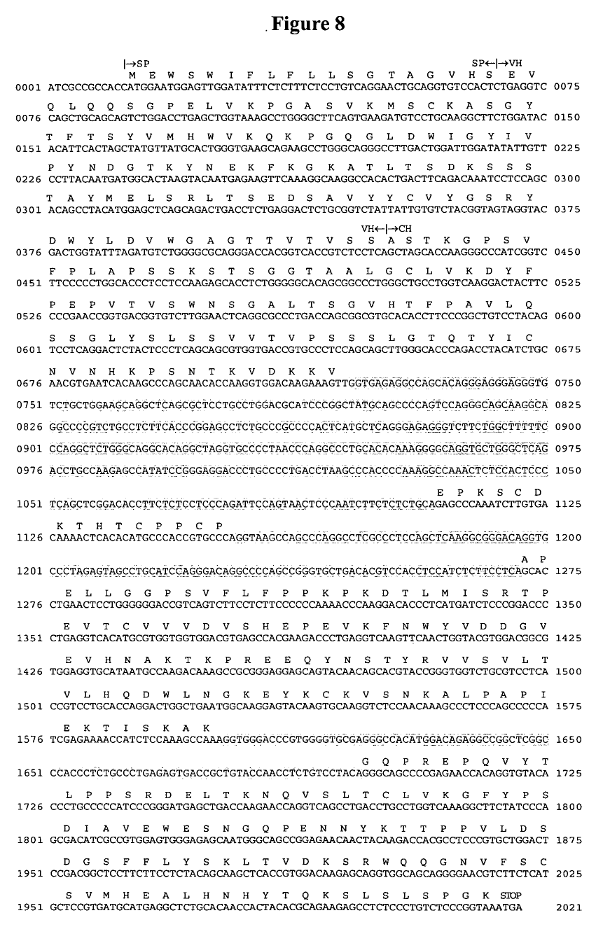

1. Cloning of mouse SM5-1 heavy and light chain variable region genes. RNA was isolated from SM5-1 (IgG1, κ) hybridoma cells (deposited at ATCC having ATCC Designation No. HB-12588) with TRIzol Reagent (Gibco BRL, Grand Island, N.Y.). The heavy and light variable region cDNAs of SM5-1 were cloned from hybridoma cells using 5′RACE system (Gibco BRL, Gaithersburg, Md.) according to the manufacture's instructions. The nested PCR products were analyzed by agarose gel electrophoresis (FIG. 5). The specific heavy chain PCR fragments of about 590 bp and light chain fragment of about 530 bp were gel-purified and cloned into pGEM-T vector (Promega, Madison, Wis.) for sequence determination, respectively. The DNA sequences of heavy (SM V_{H}) and light (SM, V_{L}) variable region are SEQ ID NO:7 (FIG. 6) and SEQ ID NO:9 (FIG. 7), respectively.

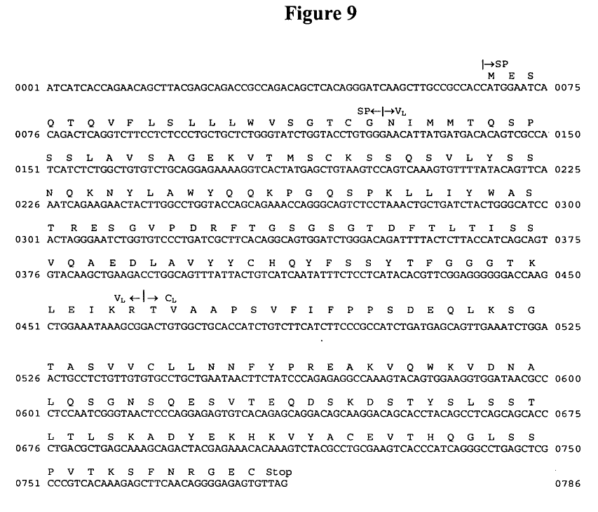

2. Construction of expression vectors for chimeric antibodies. The two vectors pAH4604 and pAG4622 were kindly provided by Prof. S L Morrison (Dept. of Microbiology and Molecular Genetics, UCLA). See Coloma et al., J Immunol Methods 152:89 (1992). Using PCR method, EcoRV and XbaI sites were added to the 5′end of the heavy chain variable region gene (V_{H}) and a NheI site added to the 3′end. The PCR product was cloned into pGEM-T vector, and its sequence was verified. The VH was excised by EcoRV and NheI digestion and inserted into the EcoRV/NheI sites of the pAH4604 vector containing the human gamma-1 constant region gene (CH). The resultant pAH4604-VH vector was cleaved with XbaI and BamHI, and the 3.3 kb fragment containing chimeric rodent/human antibody heavy chain gene cloned into the pDR vector, yielding the chimeric heavy chain expression vector PDR-SMV_{H}C_{H}. The nucleotide and deduced amino acid sequences of SM5-1 chimeric heavy chain (chSMVHCH) are shown in SEQ ID NOS:11 and 12 (FIG. 8).

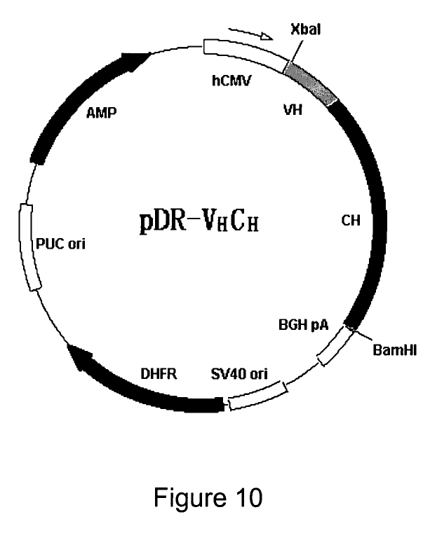

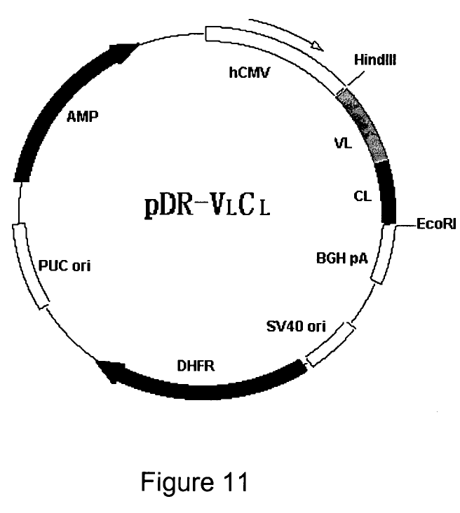

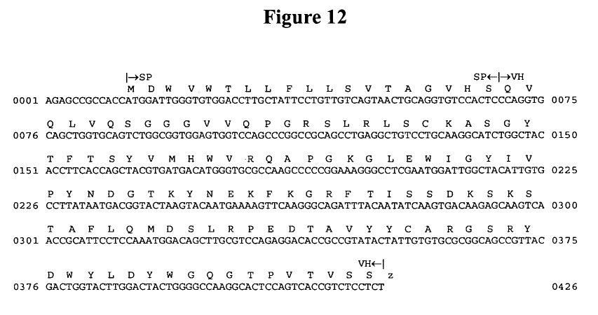

The human kappa chain constant cDNA (CL) was obtained as a 0.3 kb PCR product derived from pAG4622. pAG4622 was obtained from Prof. S. L. Morrison (Department of Microbiology and Molecular Genetics, UCLA). The light chain variable region gene (V_{L}) of SM5-1 was fused to the 5′ end of the C_{L} by overlapping PCR method. The resultant chimeric light chain gene (V_{L}C_{L}) contained a HindIII site upstream of the start codon and an EcoRI site downstream of the stop codon the chimeric light chain was cloned into pGEM-T vector gene then and its sequence was verified. The V_{L}C_{L} gene was excised by HindIII and EcoRI digestion and ligated into the pDR vector, yielding the chimeric light chain expression vector PDR-SMV_{L}C_{L}. The nucleotide and deduced amino acid sequences of SM5-1 chimeric light chain (chSMVLCL) are shown in SEQ ID NOS:13 and 14 (FIG. 9). The expression vectors PDR-SMV_{H}C_{H} and PDR-SMV_{L}C_{L} are shown in FIG. 10 and FIG. 11.

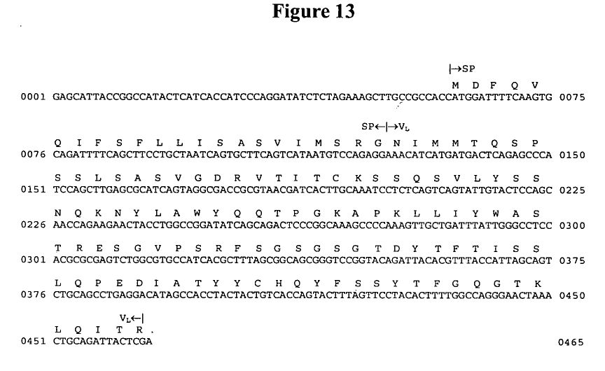

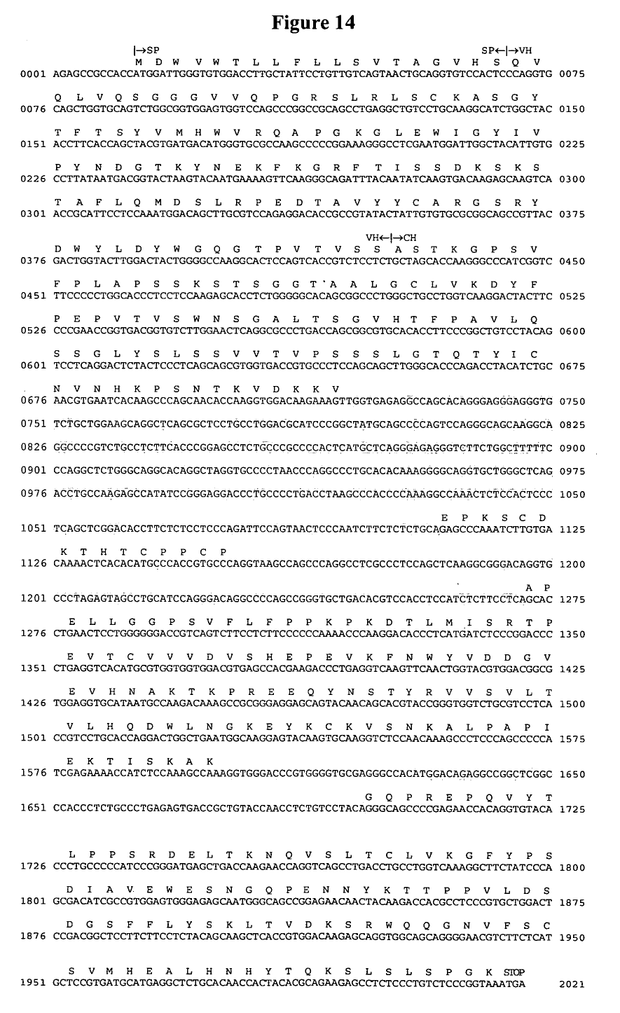

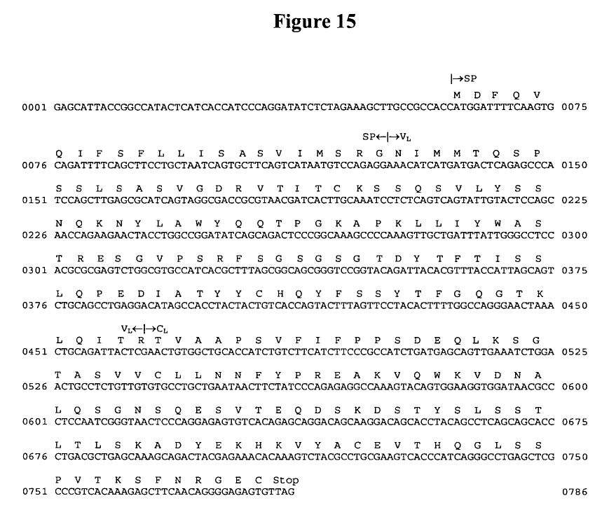

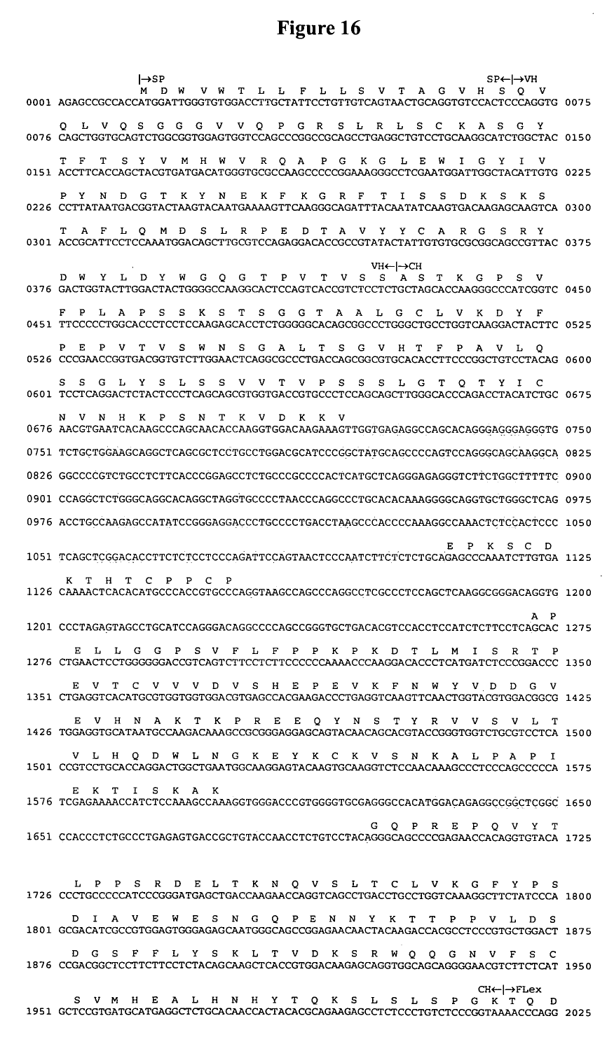

3. Construction of humanized antibody genes. The VH of human antibody KOL was chosen as framework for the humanized heavy chain and the V_{L} of human Bence-Jones protein REI was chosen as the framework for the humanized light chain. The light and heavy variable region genes of humanized antibodies were synthesized using PCR method described in Example 1. The light chain and heavy chain expression vectors for humanized antibodies were constructed in an identical manner to the chimeric antibody described above. First, nucleic acid encoding the three CDRs from SM5-1 light chain or heavy chain were directly grafted into nucleic acid encoding human antibody light chain or heavy chain framework regions to generate humanized antibody genes. The humanized V_{L} and V_{H} genes were each cloned into an expression vector and then transiently coexpressed in COS cells. The transfected COS cells produced the humanized SM5-1 Ab. Humanized antibody in the COS cell culture supernatant was quantitated by ELISA, and the binding of the antibody to melanoma cells was determined by flow cytrometric analysis. The antigen binding activity assay indicated that this antibody bound poorly to human hepatoma cell QYC, suggesting that some human FR residues must be altered to reconstitute the full binding activity. The important FR residues that may influence binding activity were analyzed, and the backmutation assay was carried out. A humanized antibody showing the same antigen binding activity as non-humanized SM5-1 was obtained and was designated “huSM.” In the competition binding assay, huSM5-1 antibody displayed equivalent avidity to the murine SM5-1 antibody and the chimeric SM5-1 antibody. The light chain and heavy chain expression vectors were denoted pDR-huSMV_{H}C_{H} and pDR-huSMV_{L}C_{L}, respectively. The nucleotide and amino acid sequences of heavy and light variable regions of huSM5-1 are shown in SEQ ID NOS:15 and 16 (FIG. 12) and SEQ ID NOS:17 and 18 (FIG. 13), respectively. The nucleotide and amino acid sequences of heavy and light chains of huSM are shown in SEQ ID NOS:19 and 20 (FIG. 14) and SEQ ID NOS:21 and 22 (FIG. 15), respectively.

4. Expression of chimeric and humanized antibodies. Prior to transfection, CHOdhfr⁻ cells were maintained in complete DMEM medium containing glycin, hypoxanthine and thymidine (GHT). Appropriate light and heavy expression vectors were cotransfected into CHOdhfr⁻ cells using Lipofectamine 2000 reagent (Invitrogen, Carlsbad, Calif.) according to the manufacture's instructions. The transfected cells were then selected in GHT free DMEM medium containing stepwise increments in MTX level up to 1.0 μM. Drug resistant clones were picked and expanded for further analysis. The culture supernatants from cell clones were analyzed for fusion protein production by the sandwich ELISA using goat anti-human IgG (Fc) (KPL) as capture antibody and goat anti-human kappa-HRP (KPL) as detector antibody. Purified human IgG1/Kappa (Sigma) was used as a standard in the ELISA assay. The clone producing the highest amount of antibody was selected and grown in serum-free medium. The recombinant antibodies were purified by Protein A affinity chromatography from the serum-free culture supernatant.

5. Affinity measurements. The affinity (Kd) of chimeric and humanized antibodies were determined using BIAcore (Pharmacia) as described Karlsson R, et al. J Immunol. Methods 145:229 (1991). The Kd values of chimeric antibody and humanized antibody are 3.78×10⁻⁹ and 9.31×10⁻⁹, respectively.

These results indicated that the humanized SM5-1 antibody possessed desirable avidity and may be used for human therapy.

### Example 4

### Construction of huSM/FL and chSM/FL Bifunctional Fusion Proteins

Three different fusion proteins were constructed for further studies of their biological function.

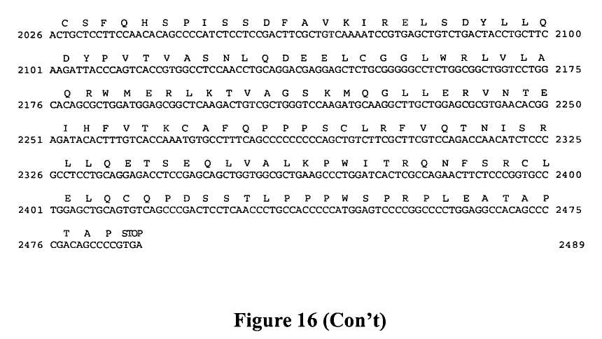

A. Construction of huSMVH/Fc/FL. Human Flt3 ligand extracellular region (hFLex) cDNA was obtained as a 500 bp PCR amplified fragment derived from pGEM-T/hFlex. The FLex gene was fused to the 3′ end of huSM heavy chain gene using overlapping PCR. The resulting fusion gene PCR product contained a HindIII site upstream of the start codon and an EcoRI site downstream of the stop codon. The fusion product was cloned into pGEM-T vector, and its sequence was verified. The nucleotide and deduced amino acid sequences of huSMV_{H}/Fc/FL are shown in SEQ ID NOS:23 and 24 (FIG. 16). Although designated as huSMV_{H}/Fc/FL, the construct also could be designated as huSMV_{H}/CH/FL or even huSMV_{H}/huγC_{H}/FL.

The huSMV_{H}/Fc/FL fusion gene was excised by HindIII and EcoRI digestion and inserted into the HindIII/EcoRI site of the pDR vector, yielding the fusion gene expression vector pDR-huSMV_{H}/Fc/FL.

Appropriate light (pDR-huSMVLCL) and fusion gene (pDR-huSMFv/Fc/FL) expression vectors were cotransfected into CHOdhfr⁻ cells using Lipofectamine 2000 reagent. The transfected cells were then selected in GHT free DMEM medium containing stepwise increments in MTX level up to 1.0 μM. Drug resistant clones were picked and expanded for further analysis. The culture supernatants from cell clones were analyzed for fusion protein production by the sandwich ELISA which used goat anti-human IgG (Fc) (KPL) as capture antibody and goat anti-human kappa-HRP (KPL) as detector antibody. Purified human IgG1/Kappa (Sigma) was used as a standard in the ELISA assay. The clone producing the highest amount of fusion protein was selected and grown in serum-free medium. The fusion protein was purified by Protein A affinity chromatography from the serum-free culture supernatant.

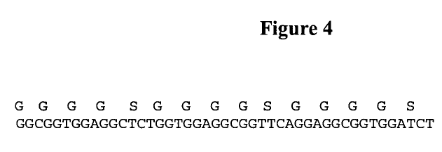

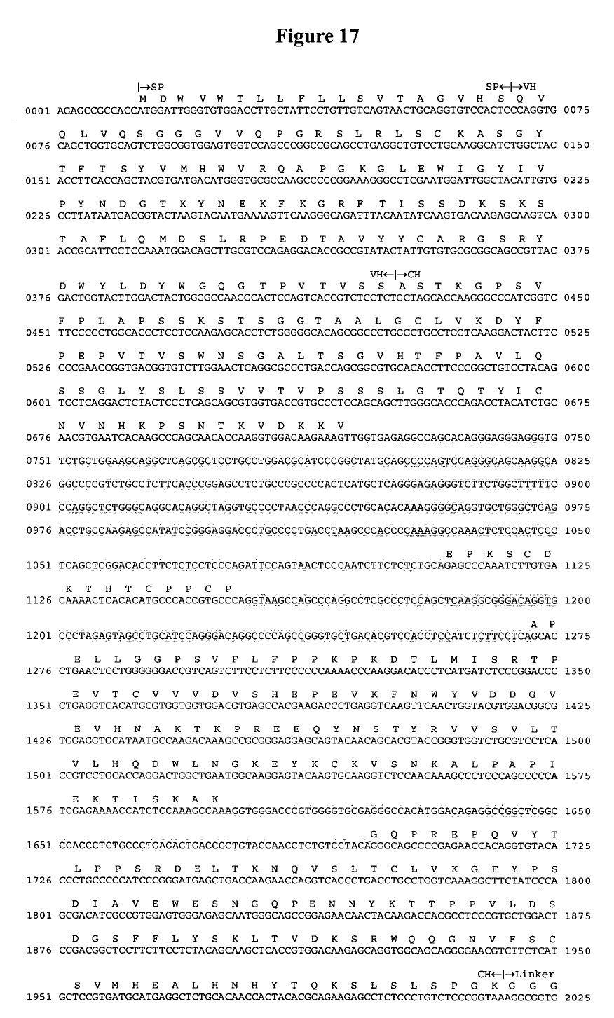

B. Construction of huSMFv/Fc/Link/FL. Human Flt3 ligand extracellular region (hFLex) cDNA was obtained as a 500 bp PCR amplified fragment derived from pGEM-T/hFlex. The hFLex gene was fused to the 3′end of huSM heavy chain gene via a linker gene by overlapping PCR. The amino acid sequence of the linker peptide is (Gly₄Ser)₃ (SEQ ID NO:6 in FIG. 4). The final PCR product containing a HindIII site upstream of the start codon and an EcoRI site downstream of the stop codon was cloned into pGEM-T vector (Promega) and its sequence was verified (shown in SEQ ID NOS:25 and 26 in FIG. 17). Although designated as huSMV_{H}/Fc/Link/FL, the construct also could be designated as huSMV_{H}/C_{H}/Link/FL or even huSMV_{H}/huγC_{H}/Link/FL.

The huSMFv/Fc/Link/FL fusion gene was excised by HindIII and EcoRI digestion and inserted into the HindIII/EcoRI site of the pDR vector, yielding the fusion gene expression vector pDR-huSVHv/Fc/Link/FL.

Appropriate light (pDR-huSMVLCL) and fusion gene (huSMVH/Fc/Link/FL) expression vectors were cotransfected into CHOdhfr⁻ cells using Lipofectamine 2000 reagent. The transfected cells were then selected in GHT free DMEM medium containing stepwise increments in MTX level up to 1.0 μM. Drug resistant clones were picked and expanded for further analysis. The culture supernatants from cell clones were analyzed for fusion protein production by the sandwich ELISA using goat anti-human IgG (Fc) (KPL) as capture antibody and goat anti-human kappa-HRP (KPL) as detector antibody. Purified human IgG1/Kappa (Sigma) was used as a standard in the ELISA assay. The clone producing the highest amount of fusion protein was selected and grown in serum-free medium. The fusion protein was purified by Protein A affinity chromatography from the serum-free culture supernatant.

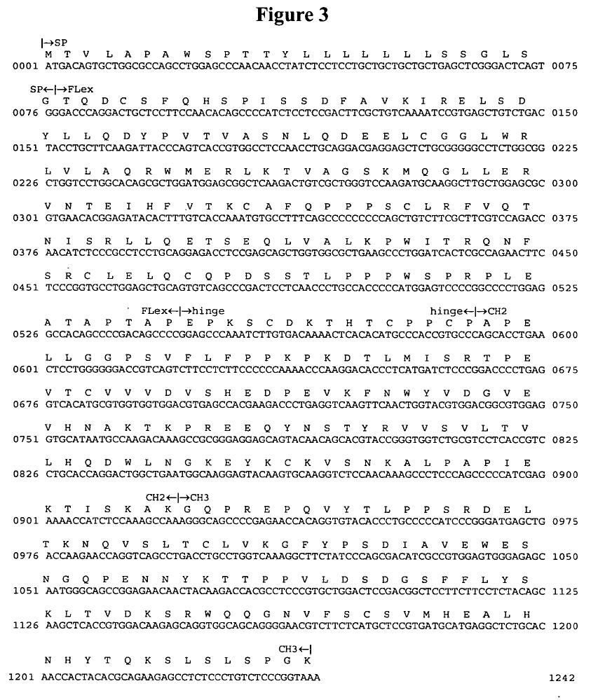

C. Construction of FL/Fc/huSMFv. Human Flt3 ligand extracellular region plus signal peptide cDNA was obtained as a 550 bp PCR amplified fragment derived from pGEM-T/hFlex. The hFLex PCR product contained a HindIII site at the 5′end, followed by a Kozak sequence to facilitate expression. The human IgG1 cDNA (hinge plus CH2 plus CH3) was amplified from pGEM-T/IgFc by PCR. The Flex gene was fused to the 5′ end of a human IgG1 cDNA using the overlapping PCR method to generate the FL/Fc fusion gene (shown in FIG. 3 SEQ ID NOS:3 and 4).

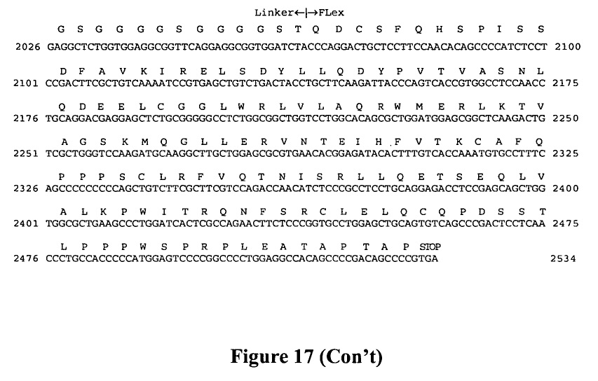

The huSM heavy chain variable region cDNA was fused to the 5′end of light chain variable region gene via a linker gene using the overlapping PCR method to generate huSM single chain antibody (ScFv) gene. The amino acid sequence of the linker peptide is (Gly₄Ser)₃ (SEQ ID NO:6). Then the FL/Fc fusion gene was fused to the 5′ end of huSM ScFv gene by overlapping PCR to generate FL/Fc/huSMFv fusion gene. The FL/Fc/huSMFv fusion gene PCR product contained a HindIII site at the 5′end and an EcoRI site downstream of the stop codon. The product then was cloned into pGEM-T vector (Promega), and its sequence was verified (shown in SEQ ID NOS:27 and 28 in FIG. 18). Although designated as FL/Fc/huSMFv, the construct also could be designated as FL/C_{H}/hUSMFv or even FL/huγC_{H}/huSMFv.

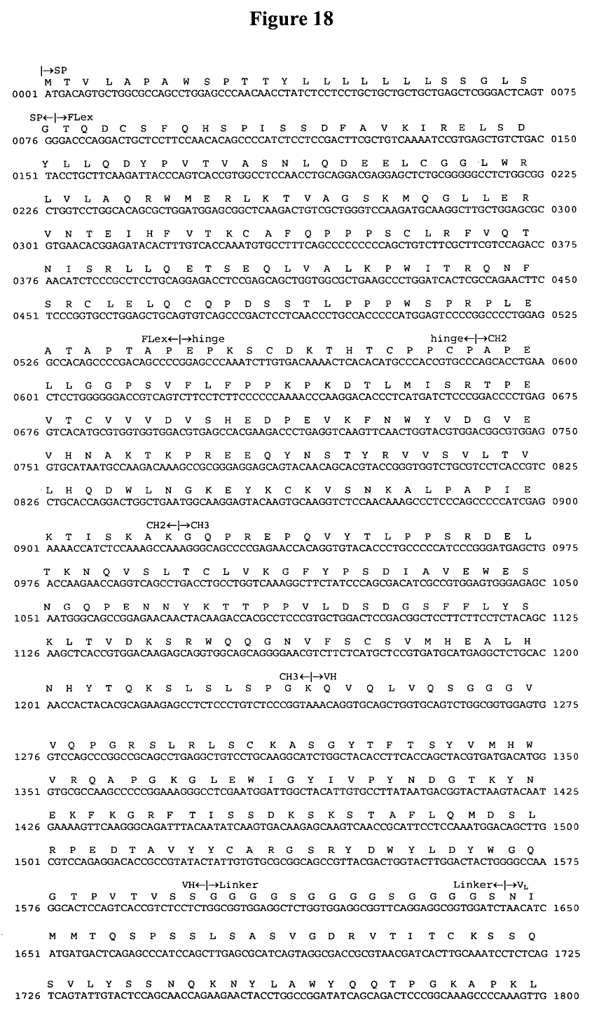

The fusion gene was excised by HindIII and EcoRI digestion and inserted into the HindIII/EcoRI site of the pDR vector, yielding the fusion gene expression vector pDR-FL/Fc/huSMFv. The schematic diagram of the FL/Fc/huSMFv fusion gene was shown in FIG. 19.

Appropriate fusion gene expression vector (pDR-FL/Fc/huSMFv) was transfected into CHOdhfr⁻ cells using Lipofectamine 2000 reagent. The transfected cells were then selected in GHT free DMEM medium containing stepwise increments in MTX level up to 1.0 μM. Drug resistant clones were picked and expanded for further analysis. The culture supernatants from cell clones were analyzed for fusion protein production by the sandwich ELISA using goat anti-human IgG (Fc) as the capture antibody and goat anti-human FLex as detector antibody. The clone producing the highest amount of fusion protein was selected and grown in serum-free medium. The fusion protein was purified by Protein A affinity chromatography from the serum-free culture supernatant.

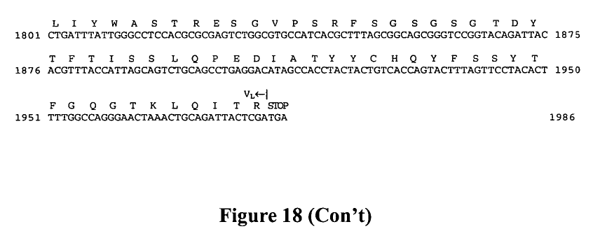

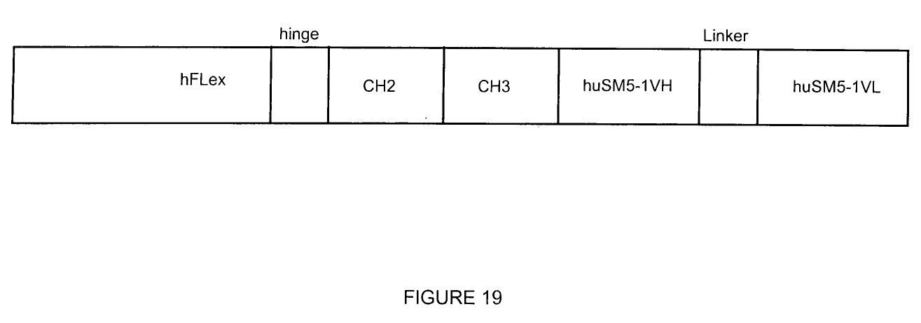

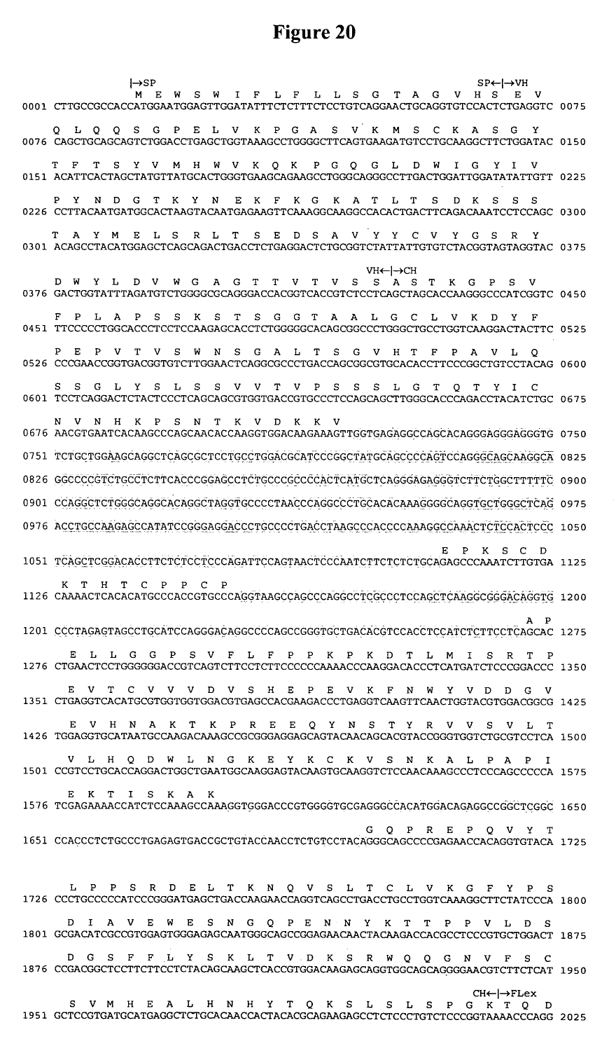

Three different ChSM/FL fusion proteins were constructed, expressed and purified in an identical manner to huSM/FL fusion proteins as described above. The nucleotide and deduced amino acid sequences of chSMV_{H}/Fc/FL, chSMV_{H}/Fc/Link/FL, FL/Fc/chSMFv are shown in SEQ ID NOS:29 and 30 (FIG. 20), SEQ ID NOS:31 and 32 (FIG. 21), and SEQ ID NOS:33 and 34 (FIG. 22), respectively.

### Example 5

### Construction of CD20/FL Bifunctional Fusion Proteins

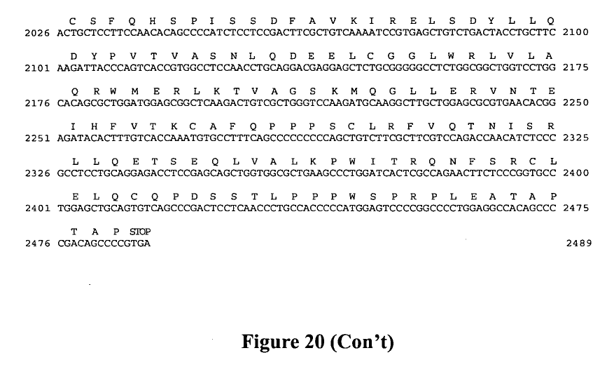

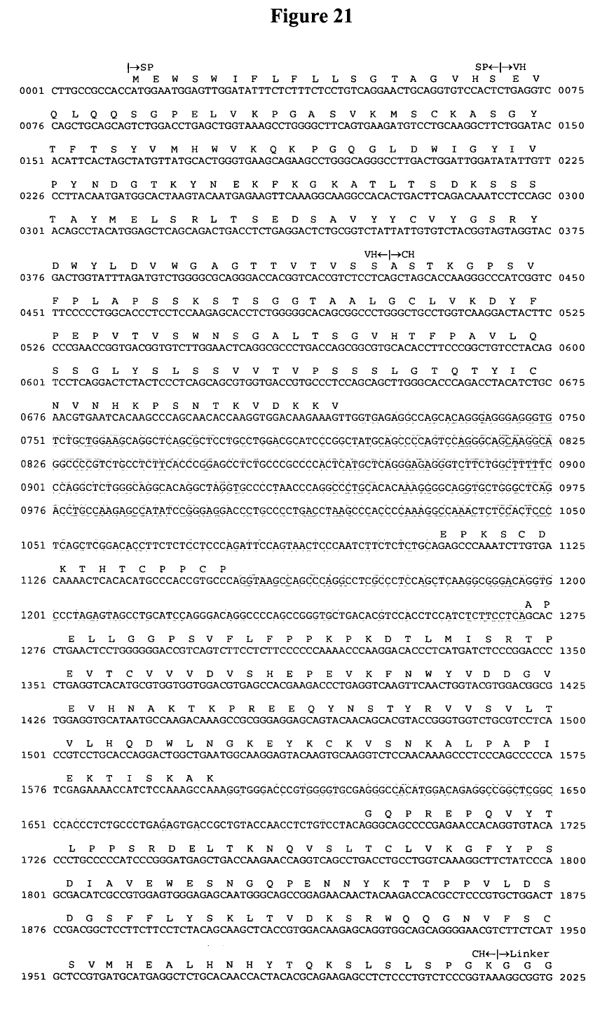

1. Synthesis of the variable region gene of anti-CD20 mAb 2B8. The variable region cDNA of ant-CD20 murine monoclonal antibody 2B8 was synthesized as described in Example 1 using the sequence disclosed in U.S. Pat. No. 6,399,061. The PCR reaction products were separated on 1% agarose gel. The correct DNA fragment was gel-purified and cloned into pGEM-T vector (Promega) and its sequence was verified. The nucleotide and amino acid sequences of heavy and light variable regions of 2B8 are shown in SEQ ID NO:35 and 36 (FIG. 23) and SEQ ID NOS:37 and 38 (FIG. 24). In this example, the correct clones for 2B8 light chain and heavy chain vectors were denoted pGEM-T/CD20H and pGEM-T/CD20L, respectively.

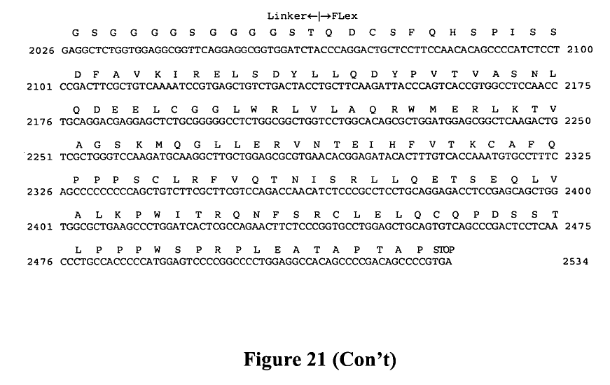

2. Construction of expression vectors for chimeric antibodies. Using PCR, EcoRV and XbaI sites were added to the 5′end of the heavy chain variable region gene (V_{H}) and a NheI site added to the 3′end. The PCR product was cloned into pGEM-T vector, and its sequence was verified. The VH was excised by EcoRV and NheI digestion and inserted into the EcoRV/NheI sites of the pAH4604 vector containing the human gamma-1 constant region gene (C_{H}). The resultant pAH4604-V_{H} vector was cleaved with XbaI and BamHI, and the 3.3 kb fragment containing chimeric rodent/human antibody heavy chain gene cloned into the pDR vector, yielding the chimeric heavy chain expression vector pDR-CD20V_{H}C_{H}. The nucleotide and amino acid sequences of anti-CD20 chimeric heavy chain (CD20V_{H}C_{H}) are shown in SEQ ID NO:39 and 40 (FIG. 25).

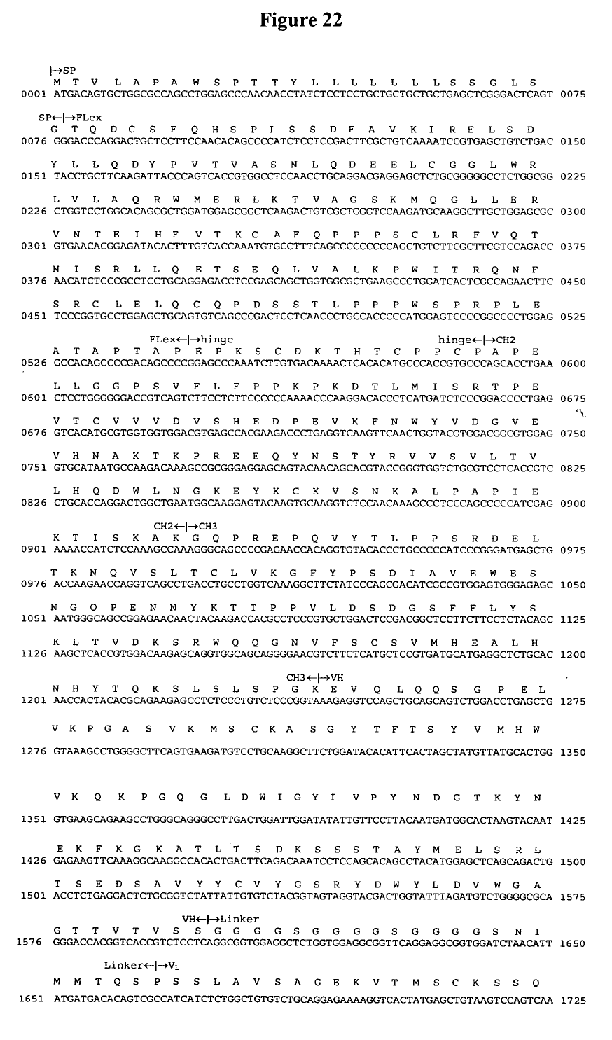

The human kappa chain constant cDNA (C_{L}) was obtained as a 0.3 kb PCR product derived from pAG4622. The light chain variable region gene (V_{L}) Of 2B8 was fused to the 5′ end of the human C_{L} by overlapping PCR method. The resultant chimeric light chain gene (V_{L}C_{L}) contained a HindIII site upstream of the start codon and an EcoRI site downstream of the stop codon the chimeric light chain was cloned into pGEM-T vector gene then and its sequence was verified. The V_{L}C_{L} gene was excised by HindIII and EcoRI digestion and ligated into the pDR vector, yielding the chimeric light chain expression vector pDR-CD20V_{L}C_{L}. The nucleotide and amino acid sequences of anti-CD20 chimeric light chain (CD20V_{L}C_{L}) are shown in SEQ ID NO:41 and 42 (FIG. 26), respectively.

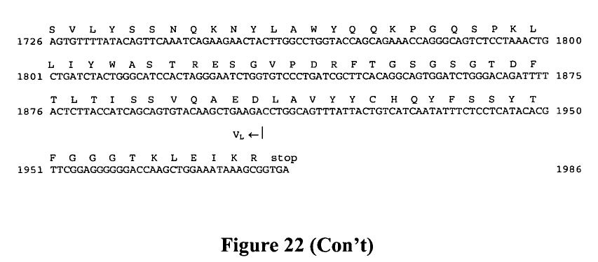

3. Construction of CD20V_{H}/Fc/FL. Human Flt3 ligand extracellular region (hFLex) cDNA was obtained as a 500 bp PCR amplified fragment derived from pGEM-T/hFlex. The FLex gene was fused to the 3′ end of 2B8 heavy chain gene by the overlapping PCR. The resulting fusion gene PCR product contained a HindIII site upstream of the start codon and an EcoRI site downstream of the stop codon. The product then was cloned into pGEM-T vector, and its sequence was verified. The nucleotide and deduced amino acid sequences of CD20V_{H}/Fc/FL are shown in SEQ ID NOS:43 and 44 (FIG. 27). The CD20V_{H}/Fc/FL fusion gene was excised by HindIII and EcoRI digestion and inserted into the HindIII/EcoRI site of the pDR vector, yielding the fusion gene expression vector pDR-CD20V_{H}/Fc/FL. Although designated as huCD20 V_{H}/Fc/FL, the construct also could be designated as hCD20V_{H}/C_{H}/FL or even huCD20V_{H}/huγC_{H}/FL.

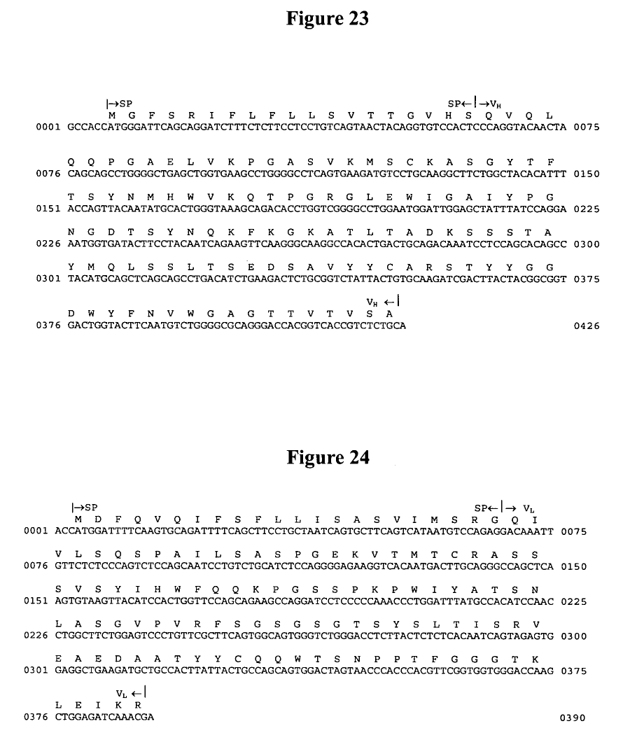

4. Construction of CD20V_{H}/Fc/Link/FL. Human Flt3 ligand extracellular region (hFLex) cDNA was obtained as a 500 bp PCR amplified fragment derived from pGEM-T/hFlex. The hFLex gene was fused to the 3′end of 2B8 heavy chain gene via a linker gene by overlapping PCR method. The amino acid sequence of the linker peptide is (Gly₄Ser)₃ (SEQ ID NO:6) The final PCR product containing a HindIII site upstream of the start codon and an EcoRI site downstream of the stop codon was cloned into pGEM-T vector (Promega), and its sequence was verified (shown in SEQ ID NOS:45 and 46 in FIG. 28). The CD20V_{H}/Fc/Link/FL fusion gene was excised by HindIII and EcoRI digestion and inserted into the HindIII/EcoRI site of the pDR vector, yielding the fusion gene expression vector pDR-CD20V_{H}/Fc/Link/FL. Although designated as huCD20 V_{H}/Fc/Link/FL, the construct also could be designated as hCD20V_{H}/C_{H}/Link/FL or even huCD20V_{H}/huγC_{H}/Link/FL.

5. Construction of FL/Fc/CD20Fv. Human Flt3 ligand extracellular region plus signal peptide cDNA was obtained as a 550 bp PCR amplified fragment derived from pGEM-T/hFlex. The hFLex PCR product contained a HindIII site at the 5′end, followed by a Kozak sequence to facilitate expression. The human IgG1 cDNA (hinge plus CH2 plus CH3) was amplified from pGEM-T/IgFc by PCR. The Flex gene was fused to the 5′ end of a human IgG1 cDNA using the overlapping method PCR to generate the FL/Fc fusion gene.

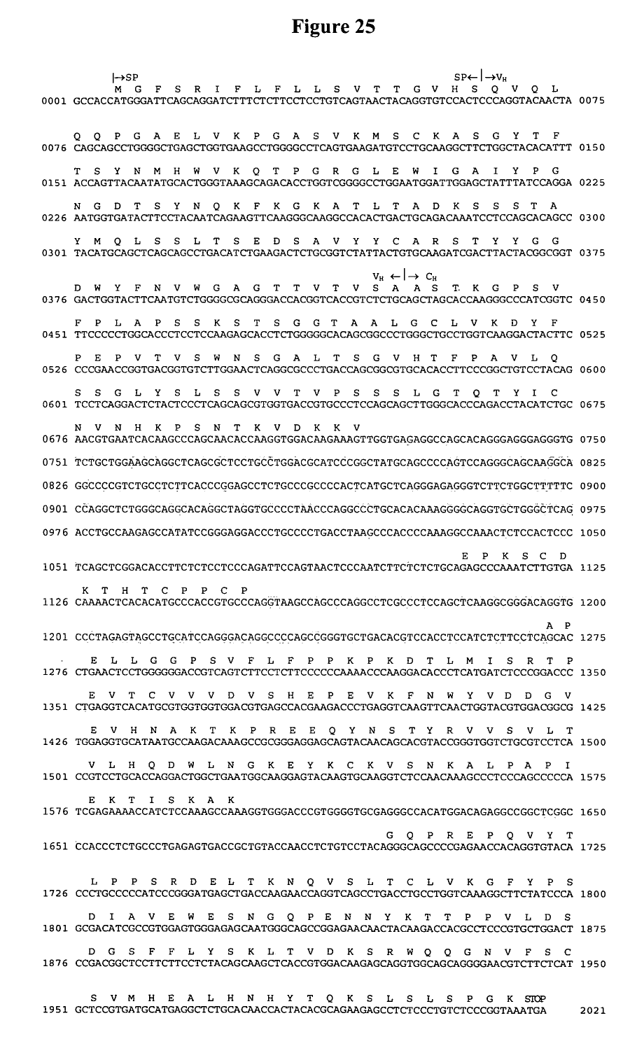

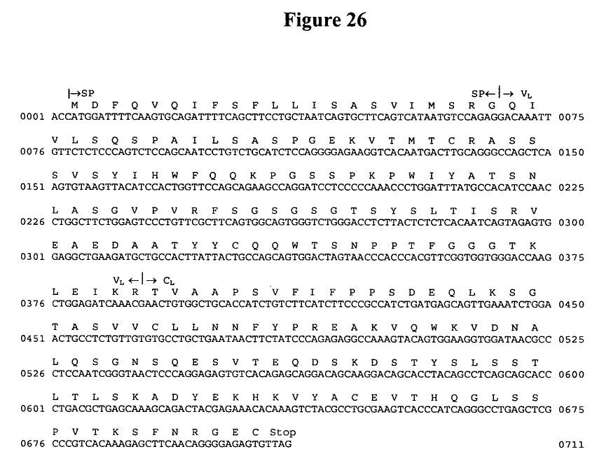

The 2B8 heavy chain variable region cDNA was fused to the 5′end of light chain variable region gene via a linker gene using the overlapping PCR method to generate 2B8 single chain antibody (ScFv) gene. The amino acid sequence of the linker peptide is (Gly₄Ser)₃. Then the FL/Fc fusion gene was fused to the 5′ end of 2B8 ScFv gene by overlapping PCR to generate FL/Fc/CD20Fv fusion gene. The FL/Fc/CD20Fv fusion gene PCR product contained a HindIII site at the 5′end and an EcoRI site downstream of the stop codon. The product then was cloned into pGEM-T vector (Promega), and its sequence was verified (shown in SEQ ID NOS:47 and 48 in FIG. 29). Then the fusion gene was excised by HindIII and EcoRI digestion and inserted into the HindIII/EcoRI site of the pDR vector, yielding the fusion gene expression vector pDR-FL/Fc/CD20Fv. The schematic diagram of the FL/Fc/CD20Fv fusion gene was shown in FIG. 30. Although designated as FL/Fc/CD20Fv, the construct also could be designated as FL/C_{H}/CD20Fv or even FL/huγC_{H}/CD20Fv.

6. Construction of 2B8 chimeric light chain expression vector. The human kappa chain constant cDNA (CL) was obtained as a 0.3 kb PCR product derived from pAG4622. pAG4622 was obtained from Prof. S L Morrison (Dept. of Microbiology and Molecular Genetics, UCLA). The light chain variable region gene (V_{L}) of SM5-1 was fused to the 5′ end of the C_{L} using the overlapping PCR method. The resultant chimeric light chain gene (V_{L}C_{L}) contained a HindIII site upstream of the start codon and an EcoRI site downstream of the stop codon the Product then was cloned into pGEM-T vector, and its sequence was verified. The V_{L}C_{L} gene was excised by HindIII and EcoRI digestion and ligated into the pDR vector, yielding the chimeric light chain expression vector pDR-CD20V_{L}C_{L}.

7. Expression and purification of fusion proteins. The three different fusion proteins were expressed and purified as described in Example 4.

### Example 6

### Construction of her2/FL Bifunctional Fusion Proteins

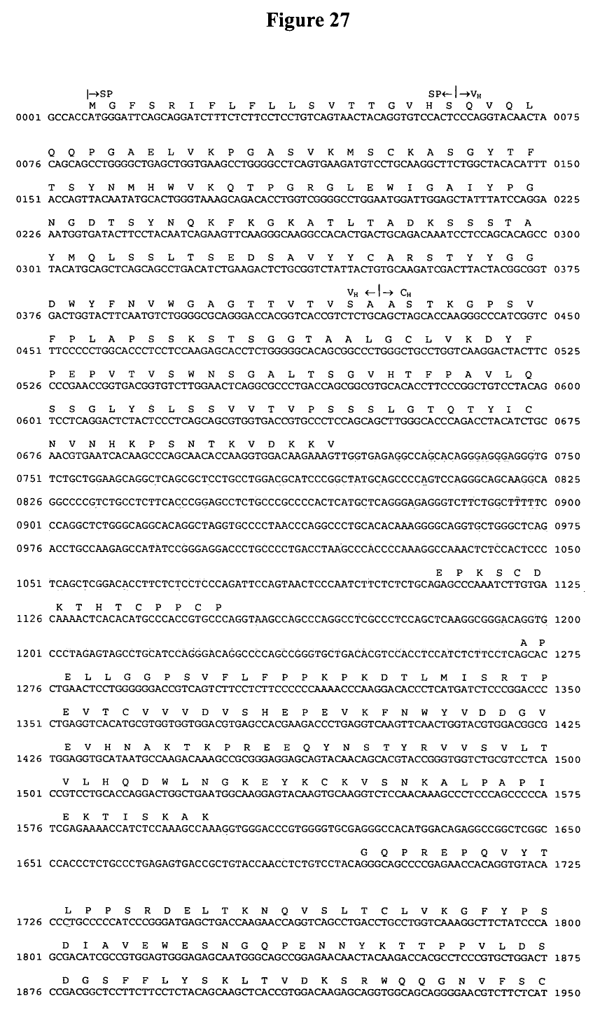

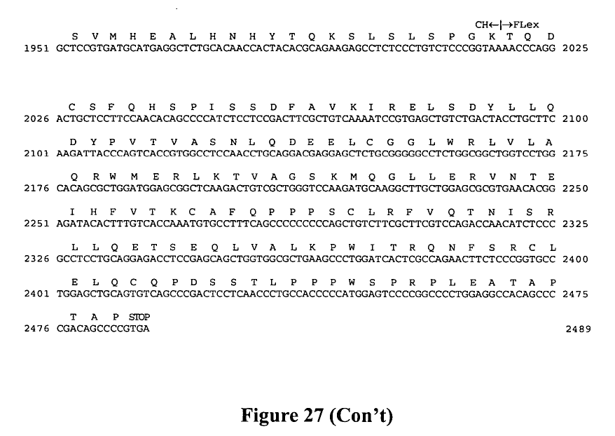

1. Synthesis of the variable region gene of anti-HER2 mAb rhuMAb HER2. The variable region cDNA of recombinant humanized ant-HER2 antibody (a.k.a. rhuMAb HER2, Herceptin) was synthesized as described in Example 1 using the sequence disclosed in Carter et al, Proc Natl Acad Sci USA, 89:4285 (1992). The PCR reaction products were separated on 1% agarose gel. The correct DNA fragment was gel-purified and cloned into pGEM-T vector (Promega), and its sequence was verified. The nucleotide and amino acid sequences of heavy and light variable regions of anti-her2 antibody are shown in SEQ ID NOS:49 and 50 (FIG. 31) and SEQ ID NOS:51 and 52 (FIG. 32), respectively. In this example, the clones for rhuMAb HER2 light chain (V_{L}) and heavy chain (V_{H}) vectors were denoted pGEM-T/her2H and pGEM-T/her2L, respectively.

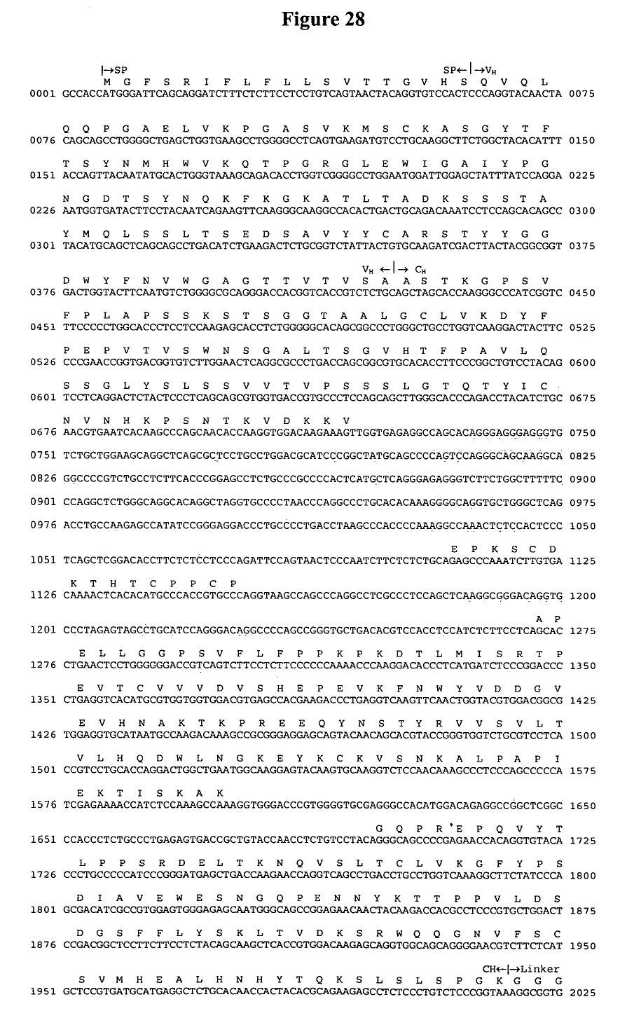

2. Construction of expression vectors for chimeric antibodies. Using PCR method, EcoRV and XbaI sites were added to the 5′end of the heavy chain variable region gene (V_{H}) and a NheI site added to the 3′end. The PCR product was cloned into pGEM-T vector, and its sequence was verified. The V_{H} was excised by EcoRV and NheI digestion and inserted into the EcoRV/NheI sites of the pAH4604 vector containing the human gamma-1 constant region gene (C_{H}). The resultant pAH4604-V_{H} vector was cleaved with XbaI and BamHI, and the 3.3 kb fragment containing chimeric rodent/human antibody heavy chain gene cloned into the pDR vector, yielding the chimeric heavy chain expression vector pDR-her2V_{H}C_{H}. The nucleotide and amino acid sequences of anti-her2 humanized heavy chain (her2V_{H}C_{H}) are shown in SEQ ID NO:53 and 54 (FIG. 33), respectively.

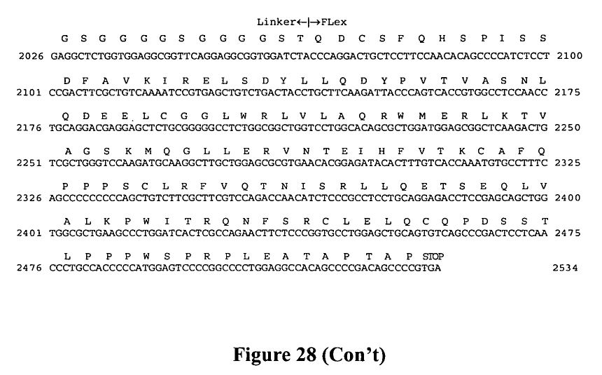

The human kappa chain constant cDNA (C_{L}) was obtained as a 0.3 kb PCR product derived from pAG4622. The humanized light chain variable region gene (V_{L}) of was fused to the 5′ end of the C_{L} by overlapping PCR method. The resultant humanized light chain gene (V_{L}C_{L}) contained a HindIII site upstream of the start codon and an EcoRI site downstream of the stop codon the humanized light chain was cloned into pGEM-T vector gene then and its sequence was verified. The V_{L}C_{L} gene was excised by HindIII and EcoRI digestion and ligated into the pDR vector, yielding the humanized light chain expression vector pDR-her2V_{L}C_{L}. The nucleotide and amino acid sequences of anti-her2 humanized light chain (her2V_{L}C_{L}) are shown in SEQ ID NOS:55 and 56 (FIG. 34).

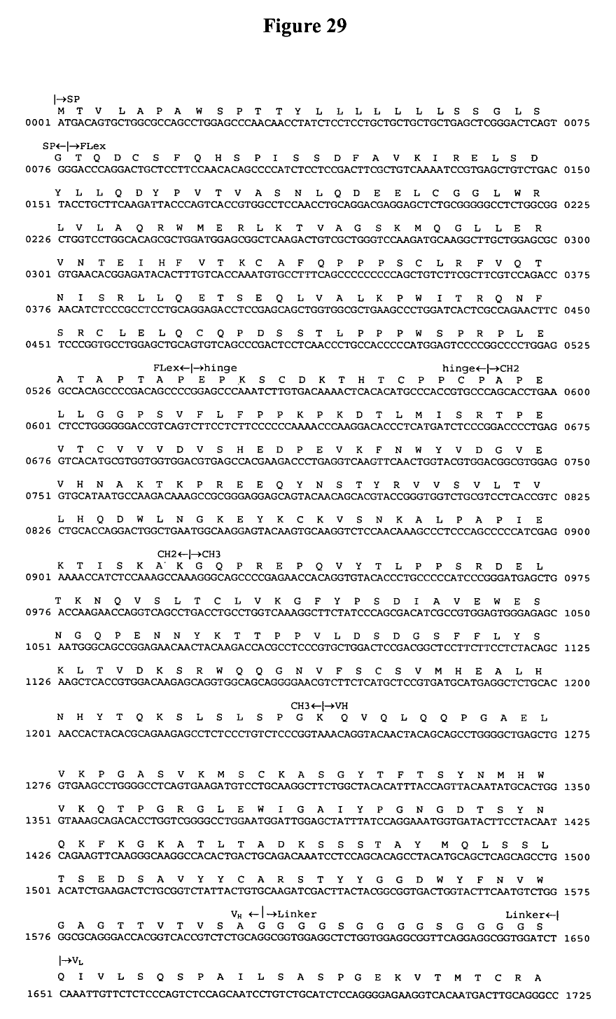

3. Construction of Her2V_{H}/Fc/FL. Human Flt3 ligand extracellular region (hFLex) cDNA was obtained as a 500 bp PCR amplified fragment derived from pGEM-T/hFlex. The FLex gene was fused to the 3′ end of rhuMAb HER2 heavy chain gene using the overlapping PCR method. The resulting fusion gene PCR product contained a HindIII site upstream of the start codon and an EcoRI site downstream of the stop codon. The product then was cloned into pGEM-T vector, and its sequence was verified. The nucleotide and amino acid sequences of Her2/Fv/Fc/FL are shown in SEQ ID NOS:57 and 58 (FIG. 35). The Her2V_{H}/Fc/FL fusion gene was excised by HindIII and EcoRI digestion and inserted into the HindIII/EcoRI site of the pDR vector, yielding the fusion gene expression vector pDR-Her2V_{H}/Fc/FL. Although designated as Her2V_{H}/Fc/FL, the construct also could be designated as Her2V_{H}/C_{H}/FL or even Her2V_{H}/huγC_{H}/FL.

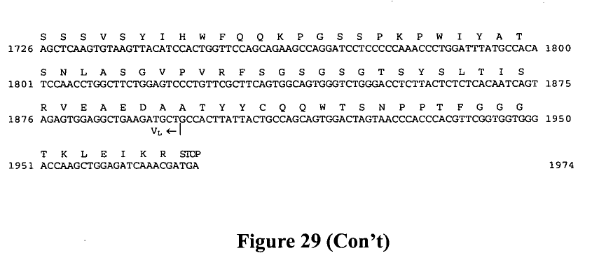

4. Construction of Her2V_{H}/Fc/Link/FL. Human Flt3 ligand extracellular region (hFLex) cDNA was obtained as a 500 bp PCR amplified fragment derived from pGEM-T/hFlex. The hFLex gene was fused to the 3′end of rhuMAb HER2 heavy chain gene via a linker gene using the overlapping PCR method. The amino acid sequence of the linker peptide is (Gly₄Ser)₃. The final PCR product contained a HindIII site upstream of the start codon and an EcoRI site downstream of the stop codon. The product then was cloned into pGEM-T vector (Promega), and its sequence was verified (shown in SEQ ID NOS:59 and 60 in FIG. 36). The Her2V_{H}/Fc/Link/FL fusion gene was excised by HindIII and EcoRI digestion and inserted into the HindIII/EcoRI site of the pDR vector, yielding the fusion gene expression vector pDR-Her2V_{H}/Fc/Link/FL. Although designated as Her2V_{H}/Fc/Link/FL, the construct also could be designated as Her2V_{H}/C_{H}/Link/FL or even Her2V_{H}/huγC_{H}/Link/FL.

5. Construction of FL/Fc/HER2Fv. Human Flt3 ligand extracellular region plus signal peptide cDNA was obtained as a 550 bp PCR amplified fragment derived from pGEM-T/hFlex. The hFLex PCR product contained a HindIII site at the 5′end, followed by a Kozak sequence to facilitate expression. The human IgG1 cDNA (hinge plus CH2 plus CH3) was amplified from pGEM-T/IgFc by PCR. The Flex gene was fused to the 5′ end of a human IgG1 cDNA using the overlapping PCR method to generate the FL/Fc fusion gene.

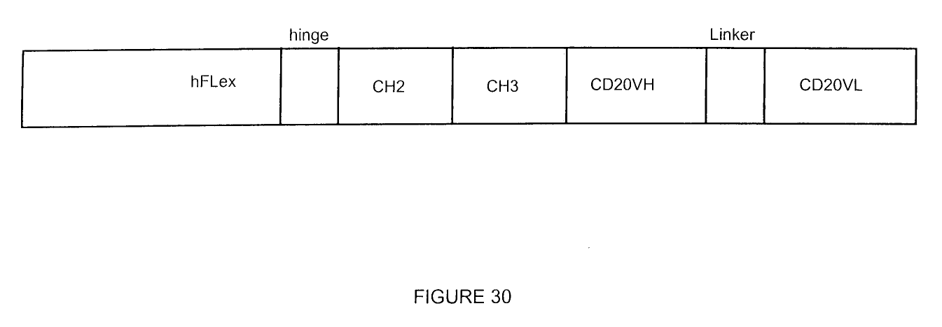

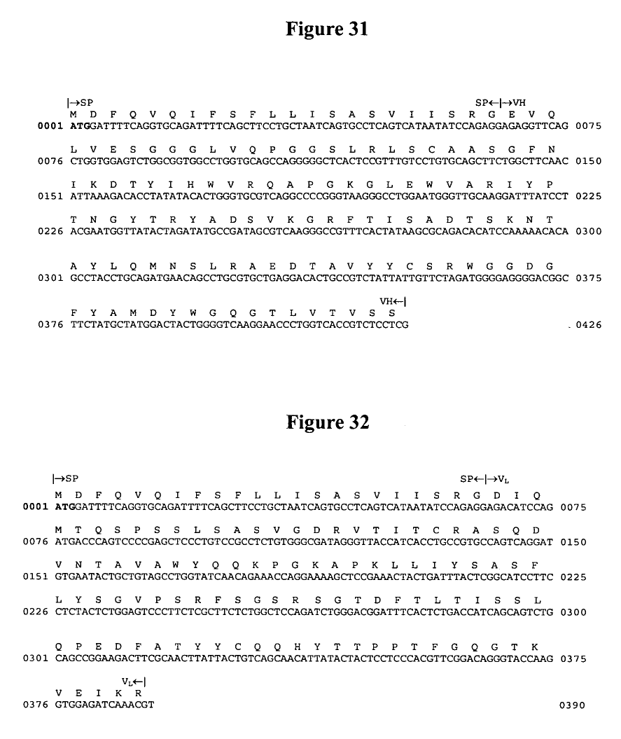

The rhuMAb HER2 heavy chain variable region cDNA was fused to the 5′end of light chain variable region gene via a linker gene using the overlapping PCR method to generate rhuMAb HER2 single chain antibody (ScFv) gene. The amino acid sequence of the linker peptide is (Gly₄Ser)₃. The FL/Fc fusion gene was fused to the 5′ end of rhuMAb HER2 ScFv gene using the overlapping PCR method to generate FL/Fc/HER2Fv fusion gene. The FL/Fc/HER2Fv fusion gene PCR product contained a HindIII site at the 5′end and an EcoRI site downstream of the stop codon. The product then was cloned into pGEM-T vector (Promega), and its sequence was verified (shown in SEQ ID NOS:61 and 62 in FIG. 37). The fusion gene was excised by HindIII and EcoRI digestion and inserted into the HindIII/EcoRI site of the pDR vector, yielding the fusion gene expression vector pDR-FL/Fc/HER2Fv. The schematic diagram of the FL/Fc/HER2Fv fusion gene was shown in FIG. 38. Although designated as FL/Fc/HER2Fv, the construct also could be designated as FL/CH/HER2Fv or even FL/huγC_{H}/HER2Fv.

6. Expression and purification of fusion proteins. The three different fusion proteins are expressed and purified as described in Example 4.

### Example 7

### Construction of hFL/Trail Fusion Protein

1. Construction of a hFLex/Trailex fusion protein. The cDNA sequence of the human FLt3 ligand gene employed has the Genbank accession number HSU37518. The extracellular domain cDNA (aa residues 95-281) for the human Trail was synthesized as described in Example 1. The PCR reaction products then were separated on 1% agarose gel. The correct DNA fragment was gel-purified and cloned into pGEM-T vector (Promega), and its sequence was verified. The clone was denoted pGEM-T/hTrail.

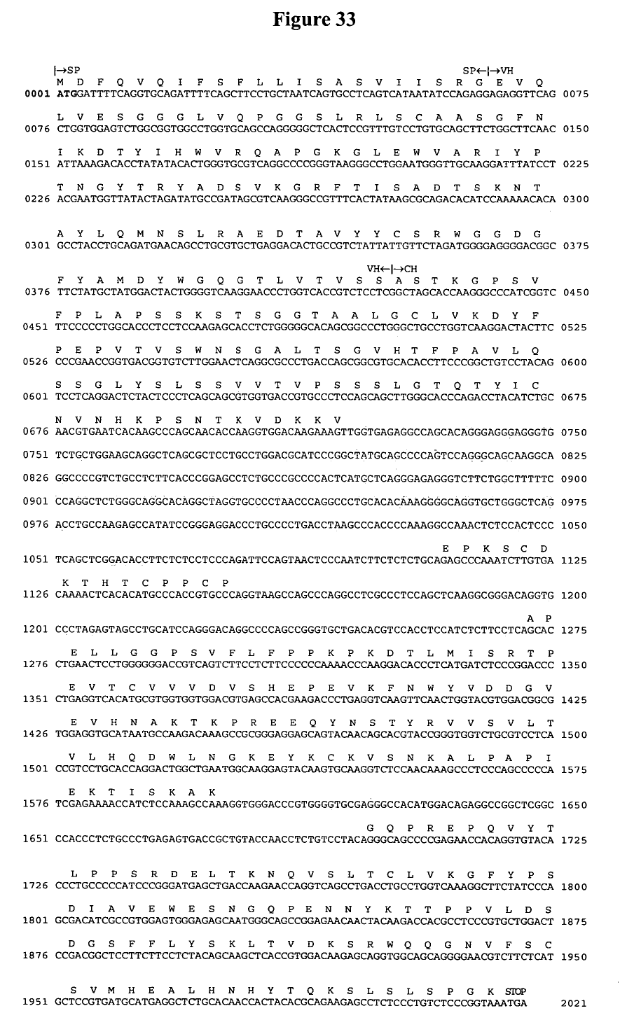

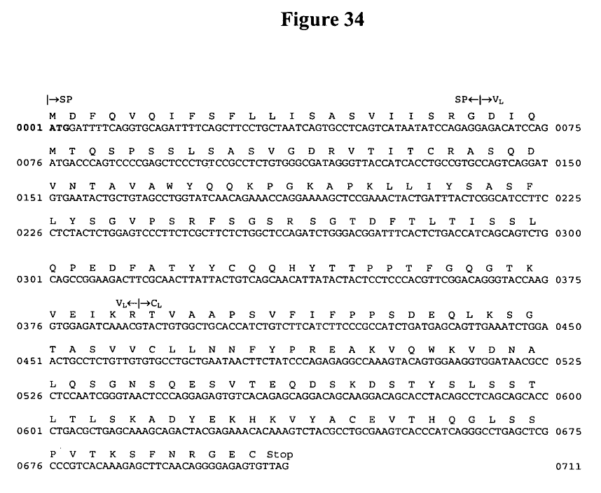

hFLex cDNA was obtained as a 550 bp PCR amplified fragment derived from pGEM-T/hFlex. The hFLex gene was fused to the 5′end of the Trailex gene (Pitti et al., J. Biol. Chem. 271:12687-90 (1996)) via a linker gene by overlapping PCR. The amino acid sequence of the linker peptide is (Gly₄Ser)₃ (SEQ ID NO:6). The fusion gene PCR product contained a HindIII site upstream of the start codon and an EcoRI site downstream of the stop codon. The product was then cloned into pGEM-T vector (Promega), and its sequence was verified (shown in SEQ ID NOS:63 and 64 in FIG. 39). The hFLex/Trailex fusion gene fragment was excised by HindIII and EcoRI digestion and inserted into the HindIII/EcoRI site of the pDR vector. The schematic diagram of the hFLex-Trailex fusion gene is shown in FIG. 40.

Appropriate pDR-hFLex/Trailex expression vector was transfected into CHOdhfr⁻ cells using Lipofectamine 2000 reagent (Gibco BRL) according to the manufacture's instruction. The transfected cells were selected in GHT free DMEM medium containing stepwise increments in MTX level up to 1.0 μM. Drug resistant clones were picked and expanded for further analysis. The culture supernatants from cell clones were analyzed for fusion protein production by the sandwich ELISA using goat anti-human Trailex as the capture antibody and goat anti-human FLex-HRP as the detector antibody. The clone producing the highest amount of fusion protein was selected and grown in serum-free medium. Then hFLex/Trailex fusion protein was purified by affinity (goat anti-human trail antibody immobilized on Sepharose-4B) from the chromatography serum-free culture supernatant.

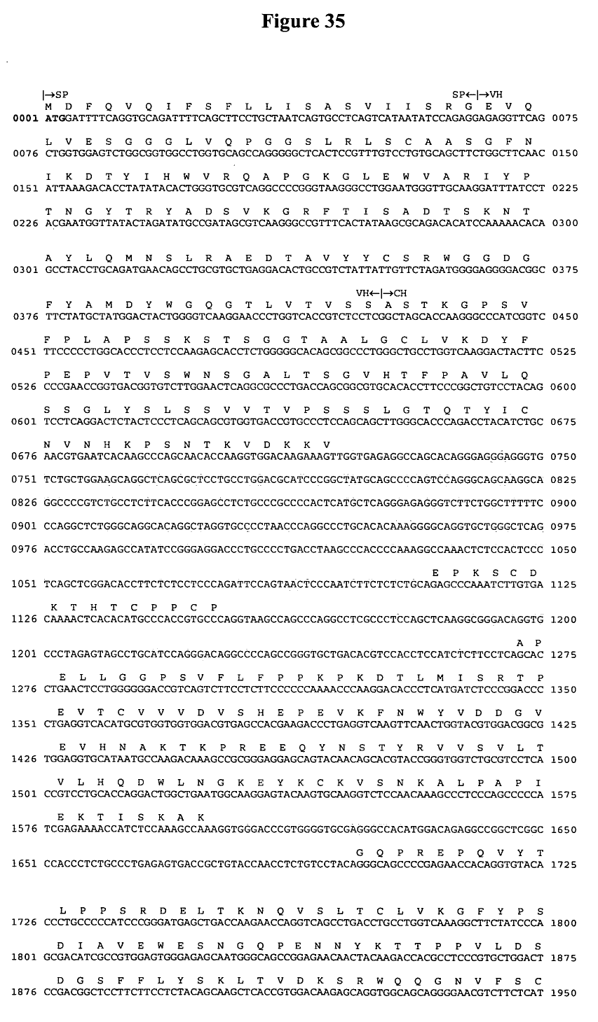

2. Construction of a hFLex/IZ/Trailex fusion protein. The hFLex gene was fused to the 5′end of the Trailex gene via a DNA sequence encoding the isoleucine zipper (IZ) by overlapping PCR. See Harbury et al. Science, 1993, 262: 1401 (1993). The fusion gene PCR product contained a HindIII site upstream of the start codon and an EcoRI site downstream of the stop codon. The product then was cloned into pGEM-T vector (Promega), and its sequence was verified (shown in SEQ ID NOS:65 and 66 in FIG. 41). The hFLex/IZ/Trailex fusion gene was finally cloned into the expression vector pGS in an identical manner to the hFLex/Trailex fusion gene described in Example 7.1. The fusion protein was expressed and purified as described in Example 7.1.

3. Construction of a hFLex/Fc/Trailex fusion protein. Human Flt3 ligand extracellular region plus signal peptide cDNA was obtained as a 550 bp PCR amplified fragment derived from pGEM-T/hFlex. The hFLex PCR product contained a HindIII site at the 5′end, followed by a Kozak sequence to facilitate expression. The human IgG1 cDNA (hinge plus CH2 plus CH3) was amplified from pGEM-T/IgFc by PCR. The Flex gene was fused to the 5′ end of a human IgG1 cDNA using the overlapping PCR method to generate the hFLex/Fc fusion gene.

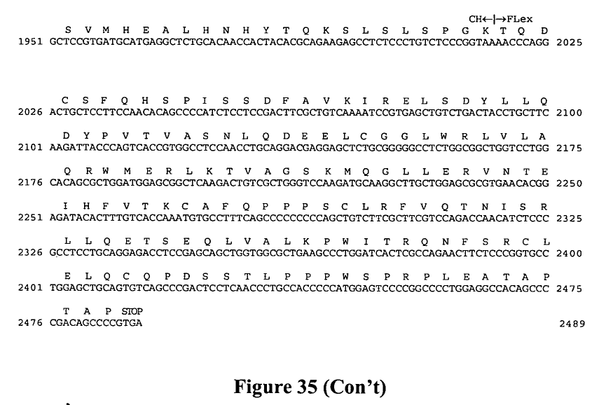

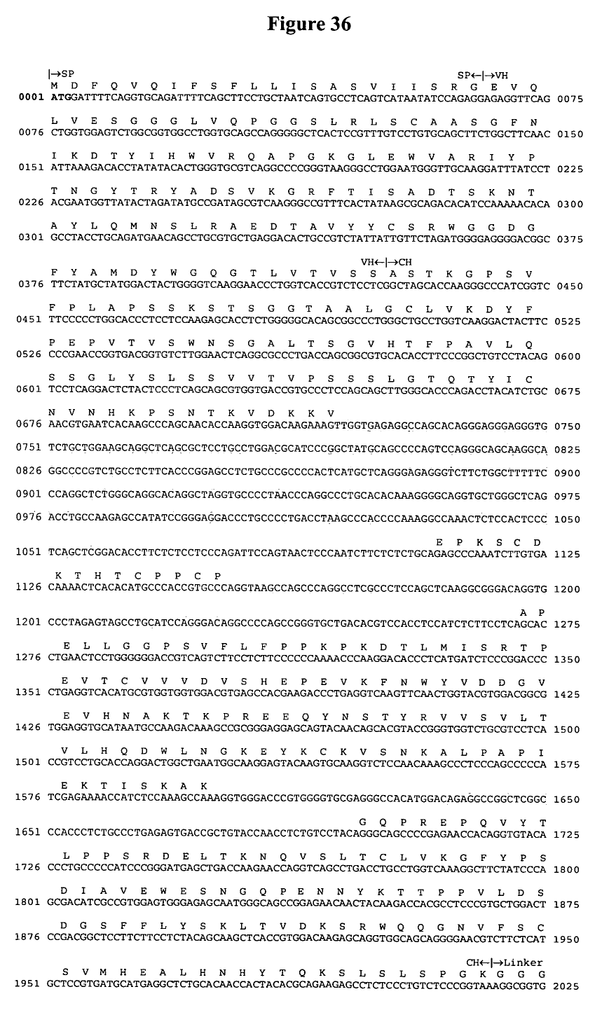

The extracellular domain cDNA of the human Trail (Trailex) was obtained from pGEM-T/hTrail by PCR amplification. The 3′end of the Trailex PCR fragment contained an EcoRI site. The hFLex/Fc fusion gene obtained previously was fused to the 5′ end of the Trailex gene using the overlapping PCR method. The final PCR product was purified and cloned into pGEM-T vector (Promega) for sequence determination (shown SEQ ID NOS:67 and 68 in FIG. 42). Then the hFLex/Fc/Trailex fusion gene fragment was excised by HindIII and EcoRI digestion and inserted into the pDR vector cleaved with the same restriction enzymes. The schematic diagram of the hFLex/Fc/Trailex fusion gene was shown in FIG. 43. Although designated as hFLex/Fc/Trailex, the construct also could be designated as hFLex/C_{H}/Trailex or even hFLex/huγC_{H}/Trailex.

Appropriate pDR-hFLex/Fc/Trailex expression vector was transfected into CHOdhfr⁻ cells using Lipofectamine 2000 reagent (Gibco BRL) according to the manufacture's instructions. The transfected cells were then selected in GHT free DMEM medium containing stepwise increments in MTX level up to 1.0 μM. Drug resistant clones were picked and expanded for further analysis. The culture supernatants from cell clones were analyzed for fusion protein production by the sandwich ELISA using goat anti-human Trail as the capture antibody and goat anti-human FL-HRP as the detector antibody. The clone producing the highest amount of fusion protein was selected and grown in serum-free medium. Then the hFLex/Fc/Trailex fusion protein was purified by Protein A affinity chromatography from the serum-free culture supernatant.

In Examples 8-16, chSM/FL, huSM/FL, CD20/FL, her2/FL, Trail/FL represent FL/Fc/chSMFv, FL/Fc/huSMFv, FL/Fc/CD20Fv, FL/Fc/HER2Fv and hFLex/IZ/Trailex, respectively.

### Example 8

### Characterization of chSM/FL(FL/Fc/chSMFv) and huSM/FL (FL/Fc/huSMFv) Bifunctional Fusion Proteins

1. Effect of SM/FL on expansion of human cord blood CD34 (+) cells in vitro. Human cord blood-derived CD34+cells were isolated using immunomagnetic beads (Pharmacia) according to the manufacture's instructions. The purity of CD34⁺ cells was analyzed by flow cytometric analysis. Cultures were set up in 0.4% agarose or 0.3% agar culture medium in the presence of 10% prescreened heat-inactivated fetal bovine serum (FBS) (Hyclone, Logan, Utah) for assessment of CFU-GM, CFU-G, OF CUR-M colonies responsive in vitro to GM-CSF, IL-3, G-CSF, SCF or CSF-1 in the absence and presence of SM or SM fusion protein. The cells were incubated at 37° C., in 5% CO₂, and the media were replaced one half one a week at the start of culture. The number of clones of CD34⁺ cells of each group were calculated at day 14.

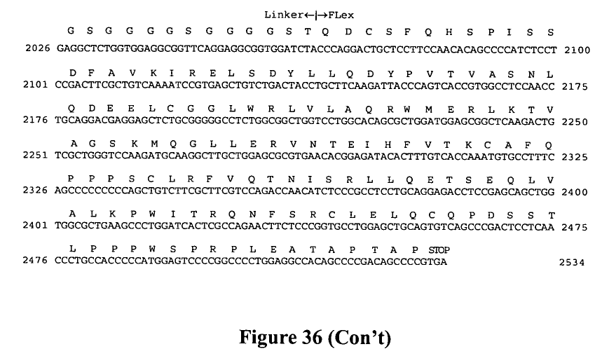

The results (shown in FIG. 44) indicated that SM/FL (chimeic or humanized SM5-1 Fv) possessed the capacity to stimulate the proliferation of CD34+ cells similar to that of FL.

2. Effects of chSM/FL and huSM/FL on NK and DC cells in vivo. C57BL/6 mice were purchased from Experimental Animal Center (Shanghai, China). FITC-conjugated anti-CD3, PE-conjugated anti-NK1.1 and FITC-conjugated anti-CD11c were obtained commercially (R&D or Sigma).

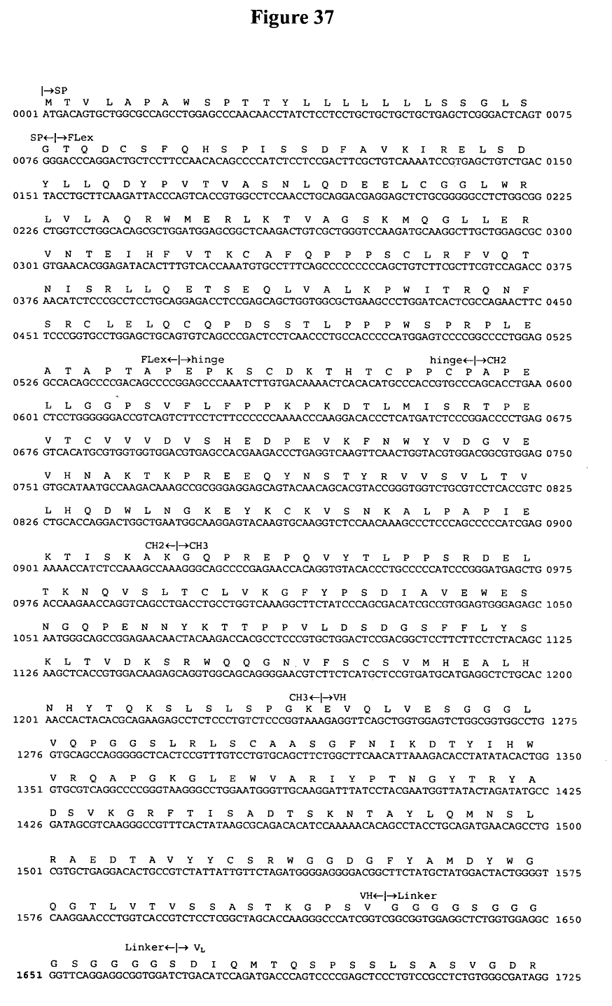

C57BL/6 mice received single injections daily of 10 μg chSM/FL and huSM/FL or FL i.p. for 0, 3, 6, 8, 10, 12, 15 or 18 days. Mice were sacrificed 24 h after the last injection. The bone marrow, spleen and liver were harvested, and single-cell suspension was prepared. Cells were two color stained with FITC-conjugated anti-CD3 and PE-conjugated anti-NK1.1 to identify NK cells. Cells were stained with FITC-conjugated anti-CD11c to identify DC cells. Flow cytometric analysis was performed to assess the percentage of NK and DC cells. The absolute numbers of NK and DC cells in each organ are shown in FIG. 45.

The results indicated that SM/FL bifunctional proteins possessed potencies to induce proliferation in NK and DC cells in spleen, liver and bone marrow comparable to FL. The numbers of NK and DC cells peaked between day 10 and 13, and the peak continued for 3 or 4 days. This suggested that SM/FL have considerable potential for the treatment of cancer.

3. Inhibition Effects of SM/FL Bifunctional Fusion Proteins on Tumor Cell Growth.

Cell lines SK-BR-3 and QYC were obtained from International Joint Cancer Institute (Shanghai, China). Cell lines Hepa1-6 and B16 were obtained from ATCC. Human melanoma cell line SMMU has been described previously (Guo et al. Cancer Res. 15;54(8):2284 (1994). QYC cells have been deposited at the American Type Culture Collection on Nov. 29, 2004 under accession no.

B16 cells were fused with QYC cells (p230 expressing) to produce hybrid cells expressing the p230 antigen. These cells are designated QYC-B16 or B16/p230. Briefly, QYC and B16 cells in logarithmic phase were fused using polyethyleneglycol and a standard hybridoma fusion protocol (QYC to B16 ratio was 1:2). The expanded hybrid cells were selected by panning against a mouse anti-SM5-1 monoclonal antibody. Briefly, the cells were added to a cell culture flask coated with the mouse anti-SM5-1 monoclonal antibody. After one hour at 37° C., the cells not bound were removed by gentle washing with 10 ml PBS. The adherent cells were eluted by elution buffer (PBS plus 0.02% EDTA) and harvested. The eluted cells were then panned against an anti-gp55 monoclonal antibody using a similar protocol as for the SM5-1 antibody. The anti-gp55 monoclonal antibody is a rat antibody prepared as described previously (Guo et al., Nat Med. 3(4):451-5 (1007)). The above double panning procedure with QYC-B16 hybrid cells was repeated 3 times.

Hepa1-6 cells were also fused with QYC cells (p230 expressing) to produce hybrid cells expressing the p230 antigen using the same protocol as described above. These cells are designated QYC-Hepa1-6 or Hepa1-6/p230. P230 was highly expressed on the cell surfaces of cell lines Hepa1-6/p230 and B16/p230 as determined by flow cytometric analysis.

Cells (SMMU, B16/p230, Hepa1-6/p230, Raji, B16, or Hepa1-6) at logarithmic growth phase were digested by 0.05% trypsin and 0.02% EDTA, and then were washed twice with PBS containing 1% FBS. The cells were resuspended in 1640/DMEM plus 10% FCS and adjusted to 6×10⁴ cells/ml. The cell suspension were added into a 96-well plate (100 ul/well) and incubated with serial dilutions of chSM/FL or huSM/FL at 37° C. in 7% CO₂ for 7 days. Proliferations of three tumor cell lines were determined using CellTiter96 AQueous non-radioactive cell proliferation assay (Promega) according to the manufacturer's instruction.

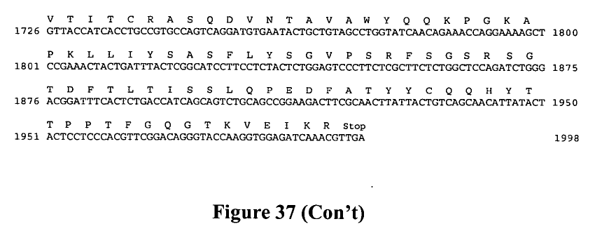

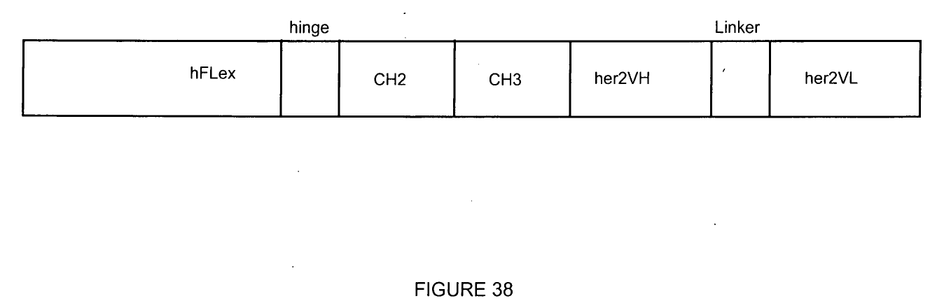

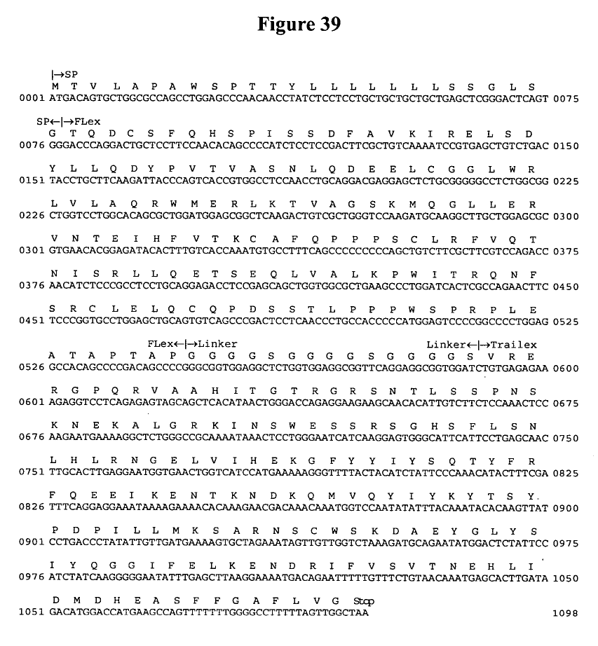

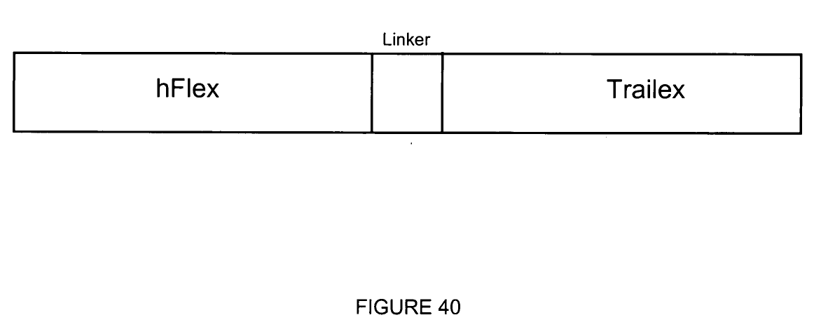

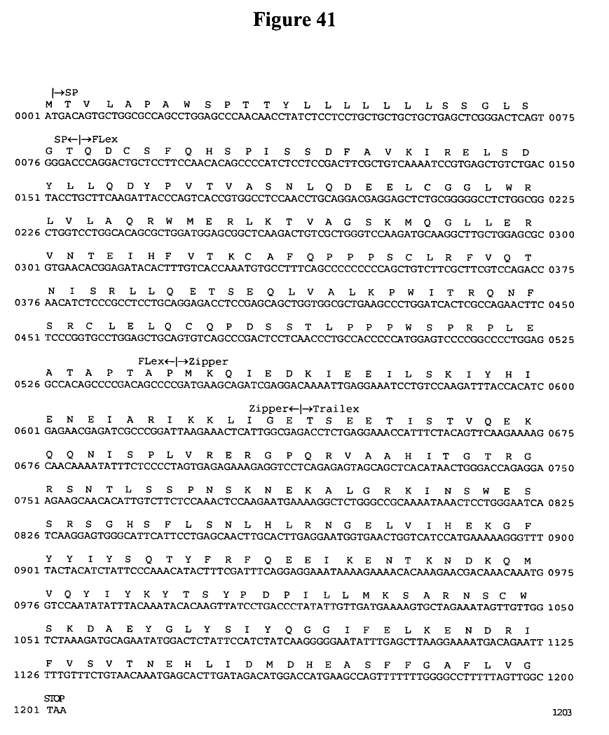

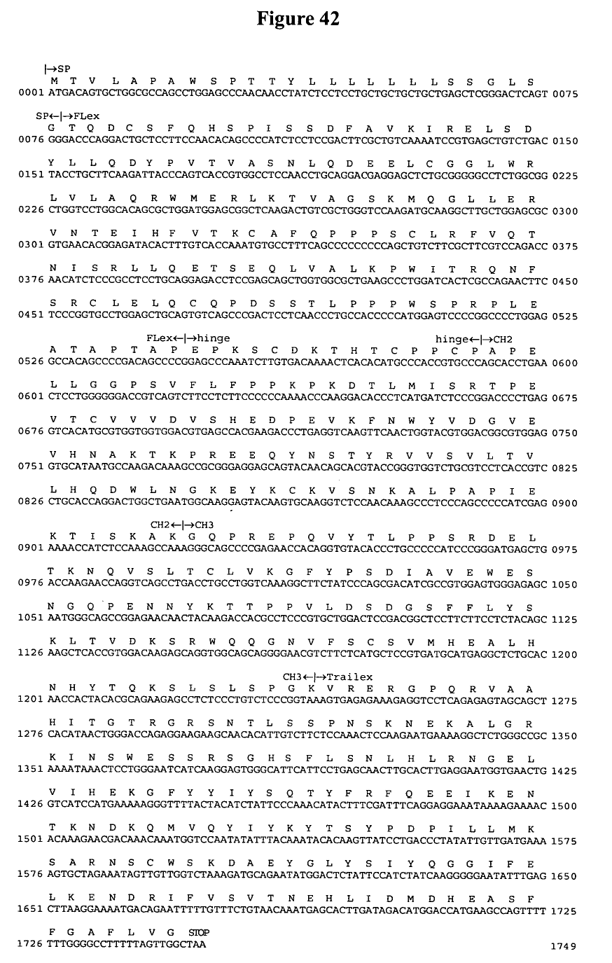

The results in FIG. 46 (A and B) with antibodies chSM/FL and huSM/FL show effective inhibition of the growth of SMMU, B16/p230, Hepa1-6/p230 tumor cells while not inhibiting the growth of control cells (Raji cells). The SM/FL chimeric proteins had no growth inhibiting effect on B 16 and Hepa1-6 cells which did not express p230 (FIGS. 47A and 47B). FIGS. 47C (B16/p230) and 47D (Hepa1-6/p230) show that p230 (but not CD3) expressing cells were growth inhibited by SM/FL but not by CD3/FL.

The results shown in FIG. 46 (A to D) indicated that chSM/FL and huSM/FL This suggested that the inhibitory effects of SM/FL were specific for tumors that express the p230 antigen.

### Example 9

### In Vitro Characterization of Her2/FL (FL/Fc/HER2Fv), CD20/FL (FL/FcCD20Fv) and Trail/FL (hFlex/IZ/Trailex)

In this experiment, the in vitro tumor inhibitory effects on tumor cells by the three bifunctional fusion proteins Her2/FL, CD20/FL and Trail/FL were evaluated. The results demonstrated that Her2/FL, CD20/FL and Trail/FL possessed potent tumor inhibitory activities similar to herceptin, rituximab and Trail, respectively.

1. Inhibition Effects of Her2/FL Bifunctional Fusion Proteins on Tumor Cell Growth.

A. Cells The cell line SK-BR-3 was obtained from International Joint Cancer Institute (Shanghai, China). The cell lines BT-474, D2F2, 4T1 were obtained from the ATCC. The cell line D2F2 was transfected with human her2 gene to create the D2F2/E2 cell line. The cell line 4T1 was transfected with her2 gene to create the 4T1her2 cell line. The her2 antigen was expressed at high levels on the cell surfaces of cell lines D2F2/E2 and 4T1her2 as determined by flow cytometric analysis.

Cells (SK-BR-3, BT-474, D2F2, 4T1, D2F2/E2 or 4T1her2) in logarithmic growth phase were digested by 0.05% trypsin and 0.02% EDTA, and then were washed twice with PBS containing 1% FBS. The cells were resuspended in 1640/DMEM plus 10% FCS and adjusted to 6×10⁴ cells/ml. The cell suspension were added into a 96-well plate (100 ul/well) and incubated with serial dilutions of her2/FL fusion proteins or positive control herceptin at 37° C., in 7% CO₂ for 7 days. The proliferation of the tumor cell lines were determined using CellTiter96 AQueous non-radioactive cell proliferation assay (Promega) according to the manufacture's instructions. The ED₅₀ values of fusion proteins or herceptin were calculated using a four parameter algorithm Y=(A−B)/[I+(X/C)^{D}]+B.

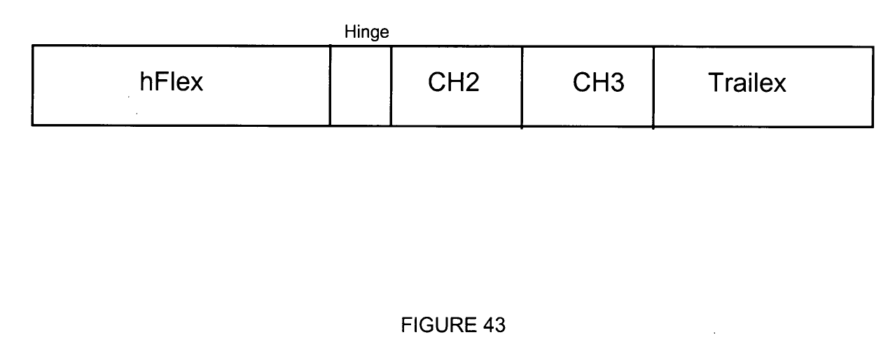

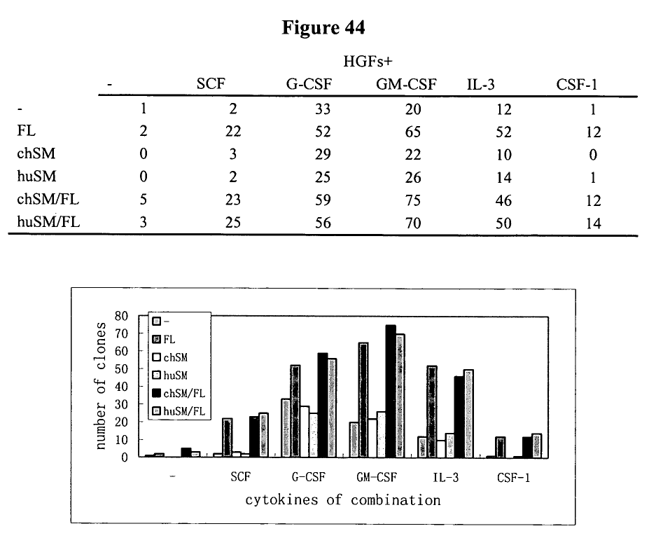

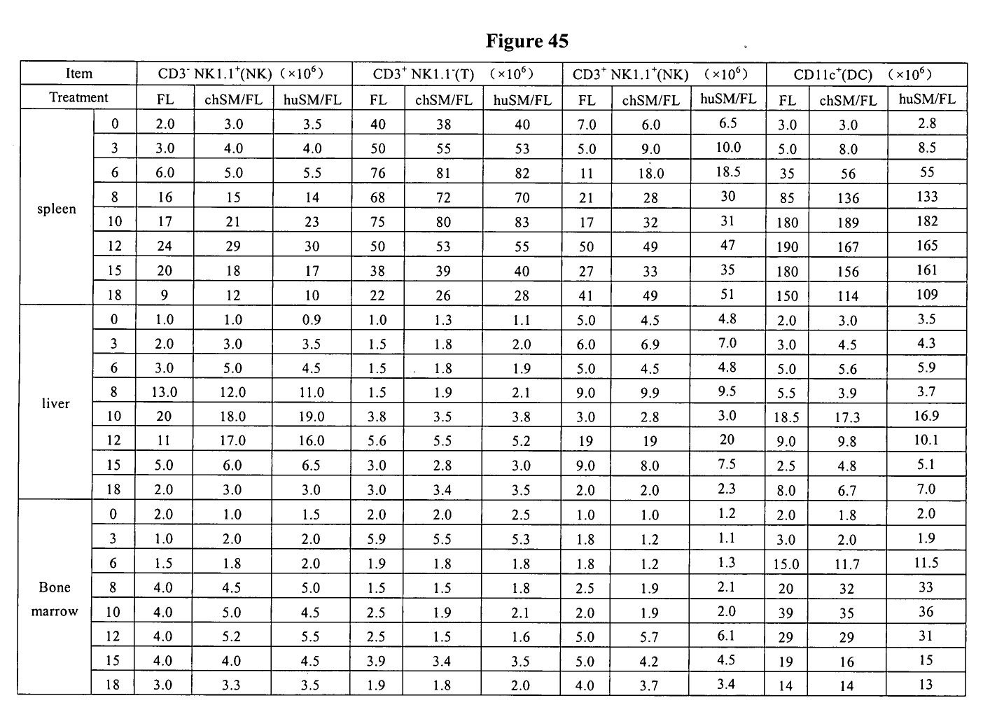

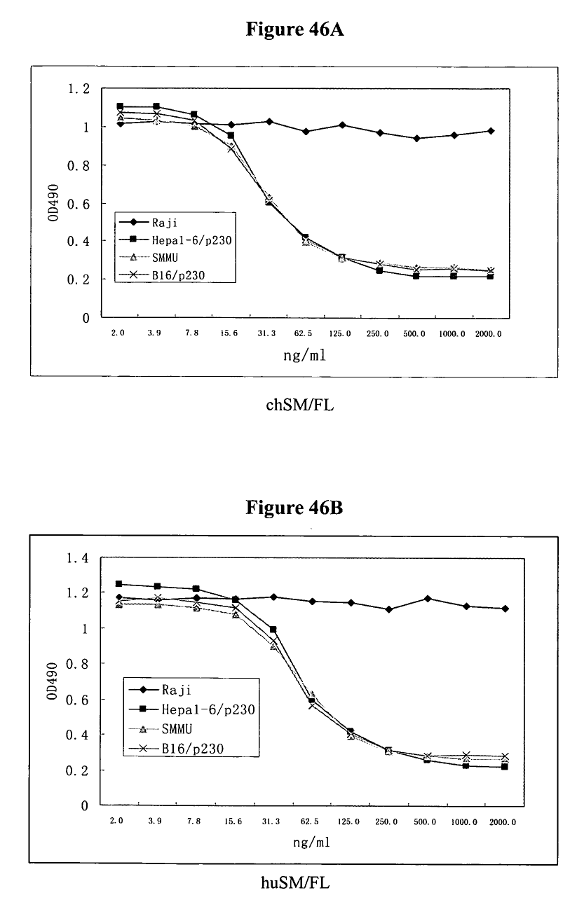

B. Cytotoxicity of Her2/FL fusion proteins on tumor cells. The results shown in FIG. 48 (A and B) indicated that her2/FL and herceptin effectively inhibited the growth of SK-BR-3, BT-474, D2F2/her2 and 4T1/her2 tumor cells. The growth of D2F2 and 4T1 cells were not inhibited by fusion proteins or herceptin. The results shown in FIG. 49 (A and B) indicated that her2/FL and herceptin effectively induced lysis of SK-BR-3, BT-474, D2F2/E2 and 4T1her2 tumor cells. Neither her2/FL nor herceptin induced the lysis of D2F2 and 4T1 cells.

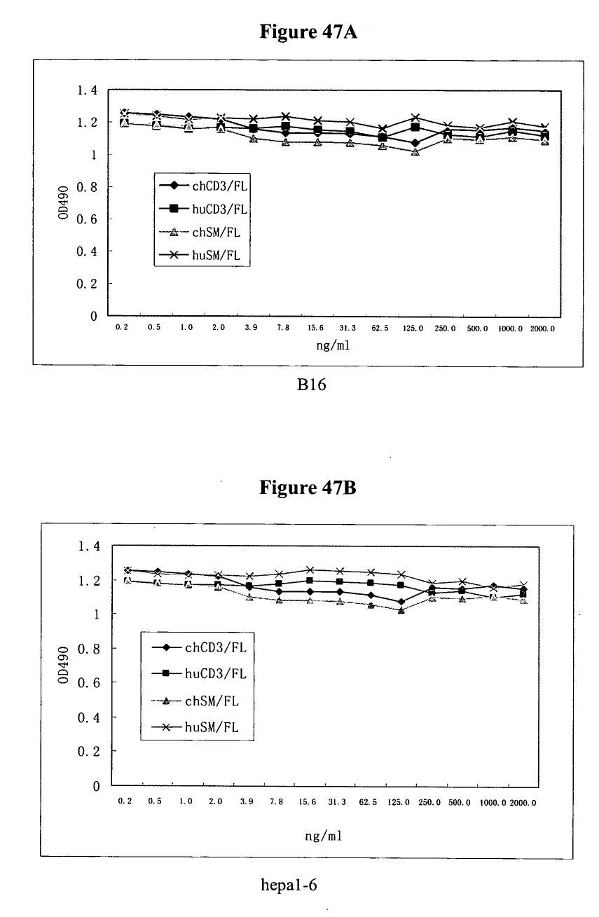

2. Cytotoxicity of CD20/FL fusion proteins on tumor cells. The Cell line Raji was obtained from the ATCC. Raji cells of logarithmic growth phase were washed twice with PBS containing 10% FBS. The cells were resuspended in 1640/DMEM plus 10% FCS and adjusted to 2×10⁵ cells/ml. The cell suspension were added into a 96-well plate (100 ul/well) and incubated with serial dilutions of CD20/FL fusion proteins or positive control rituximab at 37° C., in 7% CO₂ for 7 days. Cytotoxicity of CD20/FL and rituximab was determined using CytoTox 96 Non-Radioactive Cytotoxicity Assay (Promega) according to the manufacture's instructions. The results shown in FIG. 50 indicated that CD20/FL and rituximab effectively kill Raji tumor cells.

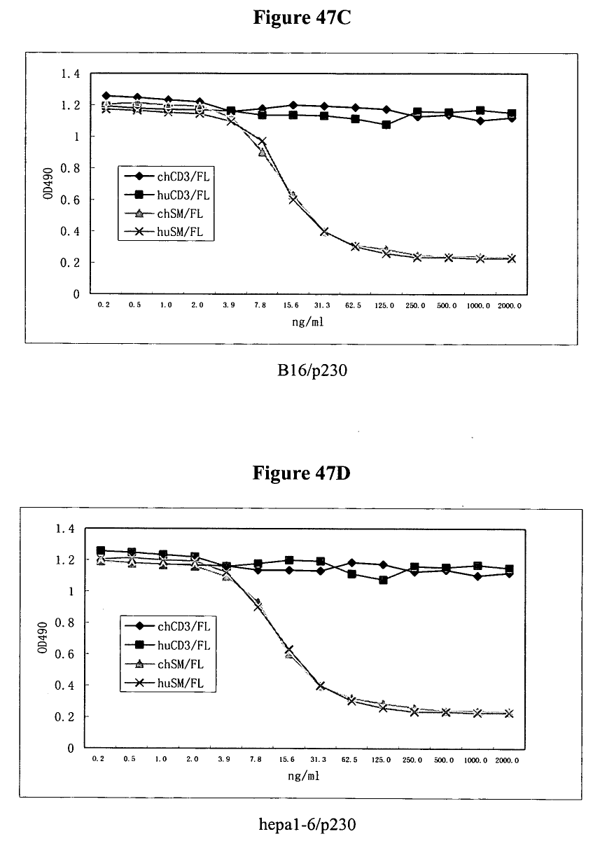

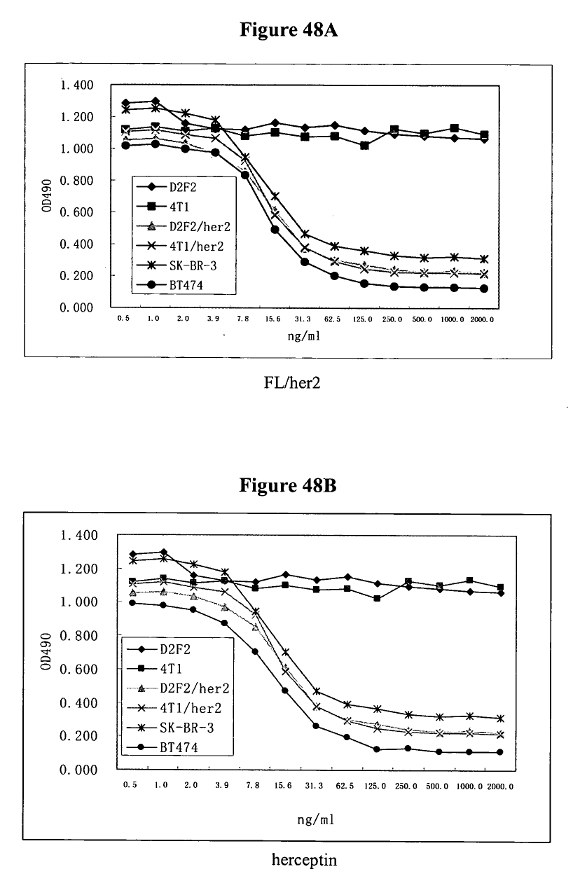

3. Inhibition effects of Trail/FL bifunctional fusion proteins on tumor cell growth. Cell lines L929, MDA-MB-231 and U-138MG were obtained from the ATCC. The cell line Renca was obtained from Korea Cancer Institute. Cells (L929, MDA-MB-231 or Renca) of logarithmic growth phase were digested by 0.05% trypsin and 0.02% EDTA, and then were washed twice with PBS containing 1% FBS. The cells were resuspended in 1640/DMEM plus 10% FCS and adjusted to 5×10⁵ cells/ml. The cell suspension was added to 96-well plates (100 ul/well) and incubated with serial dilutions of Trail/FL fusion proteins or positive control Trail at 37° C., in 7% CO₂ for 12 hours. The proliferation of the tumor cells was determined using CellTiter96 AQueous non-radioactive cell proliferation assay (Promega) according to the manufacture's instructions. The results shown in FIG. 51 (A and B) indicated that Trail/FL inhibited the growth of L929, MDA-MB-231 and Renca tumor cells similar to that of Trail. Neither Trail/FL nor Trail inhibited the growth of negative control cells U-138MG. This demonstrated that the inhibitory effects of Trail/FL and Trail were specific.

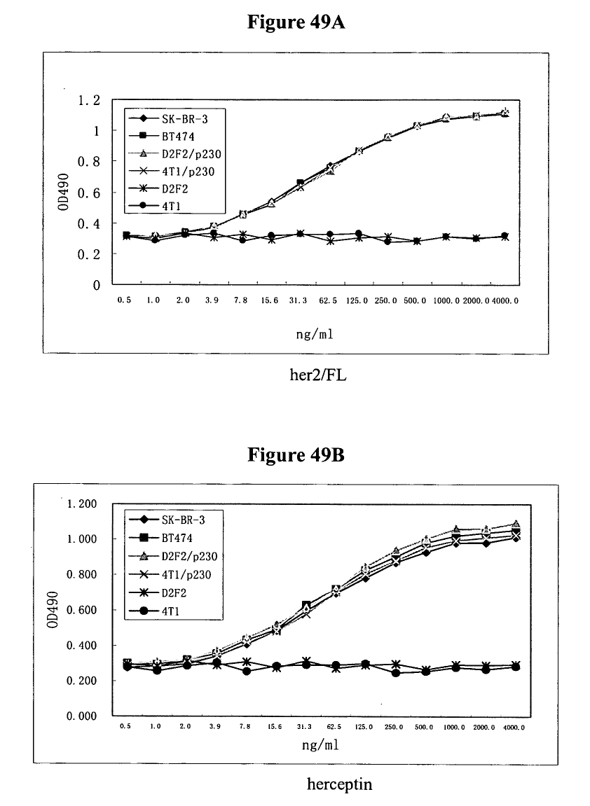

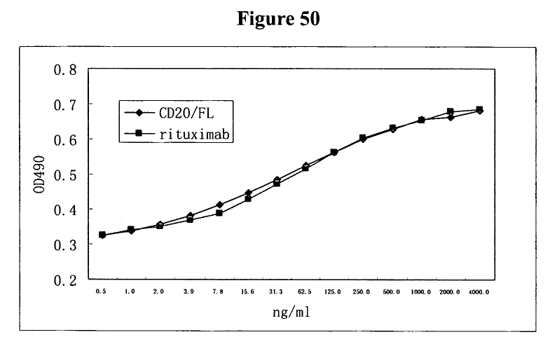

4. Cytotoxicity of Trail/FL fusion proteins. L929 and U-138MG cells of logarithmic growth phase were digested by. 0.05% trypsin and 0.02% EDTA, and then were washed twice with PBS containing 10% FBS. The cells were resuspended in 1640/DMEM plus 10% FCS and adjusted to 5×10⁵ cells/ml. The cell suspension were added into a 96-well plate (100 ul/well) and incubated with serial dilutions of Trail/FL fusion proteins or positive control Trail at 37° C., in 7% CO₂ for 14 or 16 hours. Cytotoxicity of Trail/FL and Trail was determined using CytoTox 96 Non-Radioactive Cytotoxicity Assay (Promega) according to the manufacture's instructions. The ED₅₀ values of fusion proteins or herceptin were calculated using a four parameter algorithm. The results shown in FIG. 52 (A and B) indicated that Trail/FL and Trail effectively induced the lysis of L929 cells. But neither Trail/FL nor Trail induced the lysis of control U-138MG cells.

### Example 10

### Antitumor Activities of chSM/FL and huSM/FL In Vivo

Proteins used in these experiments included: SM5-1 chimeric antibody (chSM); SM5-1 humanized antibody (huSM); chSM/FL bifunctional fusion proteins; huSM/FL bifunctional fusion proteins; anti-CD3 chimeric antibody-FL fusion proteins (chCD3/FL); anti-CD3 humanized antibody-FL fusion proteins (huCD3/FL).

Female C57BL/6 mice were subcutaneously injected with B16, Hepa1-6, B16p230 or hepap230 tumor cells. When tumors reached 0.5 cm in diameter, the mice were randomized into seven groups with ten mice each. Six groups of mice were injected i.v. with chCD3/FL, huCD3/FL, chSM, huSM, chSM/FL or huSM/FL at a dose of 4 mg/kg/week for 6 consecutive weeks. The group of mice injected i.v. with PBS was the negative control group. Tumor regression was observed after treatment.

The experimental results (shown in Table 3) indicated that chSM, huSM, chSM/FL and huSM/FL effectively induced the regression of tumor expressing p230 antigen. The FL fusion proteins significantly enhanced the antitumor activities of chSM or huSM antibodies.

TABLE 3
Tumor regression after treatment.

| | Anti CD3/FL | | | | Anti SM5-1/FL fusion | | |
|---|---|---|---|---|---|---|---|
| | fusion protein | | Anti SM5-1 antibody | | protein | | |
| Cell line | chimeric | humanized | chimeric | humanized | chimeric | humanized | PBS |
| Hepa1-6 | 0/10 | 0/10 | 0/10 | 0/10 | 0/10 | 0/10 | 0/10 |
| Hepa1- | 0/10 | 0/10 | 7/10 | 8/10 | 10/10 | 10/10 | 0/10 |
| 6/p230 | | | | | | | |
| B16 | 0/10 | 0/10 | 0/10 | 0/10 | 0/10 | 0/10 | 0/10 |
| B16/p230 | 0/10 | 0/10 | 8/10 | 7/10 | 10/10 | 10/10 | 0/10 |

### Example 11

### Specific Tumor Immune Responses Induced by chSM/FL and huSM/FL In Vivo

The proteins used in these experiments include: SM5-1 chimeric antibody (chSM); SM5-1 humanized antibody (huSM); chSM/FL bifunctional fusion proteins; huSM/FL bifunctional fusion proteins; anti-CD3 chimeric antibody-FL fusion proteins (chCD3/FL); and anti-CD3 humanized antibody-FL fusion proteins (huCD3/FL).

Female C57BL/6 mice were subcutaneously injected with B16p230 or hepap230 tumor cells. When tumors reached 0.5 cm in diameter, mice were randomized into seven groups with 8 mice each. Six groups of mice were injected i.v. with chSM, huSM, chSM combined with FL, huSM combined with FL, chSM/FL or huSM/FL at a dose of 4 mg/kg/week for 6 consecutive weeks. The group of mice injected i.v. with PBS was the negative control group. Tumor regression was observed after treatment.

The experimental results (shown in Table 4) indicated that the administration of chSM (or huSM) combined with FL exhibited antitumor activities than chSM (or huSM) alone. The bifunctional fusion protein chSM (or huSM) exhibited the strongest antitumor activity in this study.

TABLE 4
Anti-tumor activities of bifunctional fusion proteins.

| | | absence of tumor outgrowth |
|---|---|---|
| treatment | Tumor regression | from the second challenge |
| chSM | 5/10, 5/10, 6/10 | 0/10 |
| huSM | 4/10, 5/10, 4/10 | 0/10 |
| chSM + FL | 6/10, 6/10, 8/10 | 6/10 |
| huSM + FL | 7/10, 5/10, 6/10 | 6/10 |
| chSM/FL | 10/10, 8/10, 10/10 | 28/30 |
| huSM/FL | 10/10, 10/10, 10/10 | 30/30 |

To determine whether fusion protein-induced tumor regression resulted in the generation of an active anti-tumor immune response, mice (e.g., receiver fusion proteins i.v.) were inoculated again to challenge with parental tumor cells subcutaneously, e.g., either B16p230 or hepap230 cells. Tumor regression was observed after inoculation. The results (shown in Table 5) indicated that chSM or huSM did not induce an active anti-tumor immune response. However, both chSM/FL and huSM/FL elicited an active anti-tumor immune response against parental tumor, resulting in the absence of tumor outgrowth from the second challenge of tumor cells. These results demonstrated that the antitumor immune responses induced by bifunctional fusion proteins were specific for the tumor given in the challenge.

TABLE 5
Induction of active anti-tumor immune response by bifunctional
fusion proteins

| | | absence of tumor outgrowth from | |
|---|---|---|---|
| | | the second challenge | |
| Cell line | Treatment | B16 | Hepa1-6 |
| B16/p230 | chSM/FL | 0/6 | 5/6 |
| B16/p230 | huSM/FL | 0/6 | 5/5 |
| Hepa1-6/p230 | chSM/FL | 5/5 | 0/5 |
| Hepa1-6/p230 | huSM/FL | 5/5 | 1/5 |

### Example 12

### Antitumor Activities of Her2/FL, CD20/FL, Trail/FL Fusion Proteins In Vivo

To study the in vivo anti-tumor activities of bifunctional fusion proteins which were constructed by fusing FL to other antibodies or molecules that could induce the apoptosis of tumor cells, the following experiments were done. Experimental results demonstrated that the bifunctional fusion proteins constructed by fusing FL to anti-her2 mAb, anti-CD20 mAb or Trail were all inhibitory to tumor growth.

1. Antitumor activities of her2/FL in vivo. Human breast carcinoma cell line BT474 was obtained from the ATCC. Male Balb/c nude mice were obtained from Experimental Animal Center (Shanghai, China).

Balb/c nude mice were subcutaneously injected with 5×10⁶ BT-474 tumor cells. When tumors reached 0.5 cm in diameter, mice were randomized into experimental and control groups with ten mice each. Experimental group of mice were injected i.v. with her2/FL at a dose of 10 mg/kg/week for 6 consecutive weeks. The control group of mice were injected i.v. with PBS. Continuous tumor growth was observed in all animals for 6 weeks.

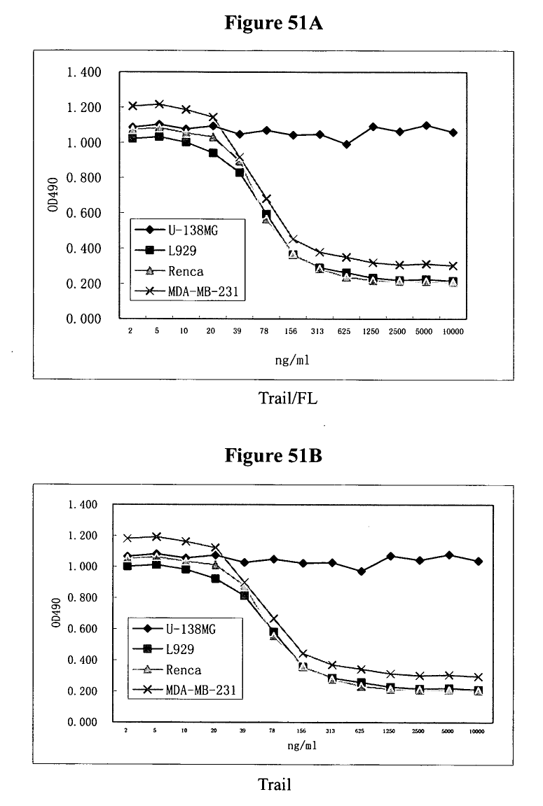

Statistical analysis of the differences was performed using the Student's t test. The results (shown in FIG. 53) indicated that treatment with her2/FL fusion protein possessed highly significant anti-tumor activity (p≦0.038).

2. Anti-tumor activities of CD20/FL in vivo. The Cell line Raji was obtained from the ATCC. Female Balb/c nude mice were obtained from Experimental Animal Center (Shanghai, China).

Balb/c nude mice were irradiated with 2GY once a week for 3 consecutive weeks. The irradiated nude mice were then subcutaneously injected with 2×10⁷ Raji tumor cells. When tumors reached 0.5 cm in diameter, mice were randomized into experimental and control groups with ten mice each. Experimental group of mice were injected i.v. with CD20/FL at a dose of 10 mg/kg/week for 6 consecutive weeks. The control group of mice were injected i.v. with PBS. Continuous tumor growth was observed in all animals for 6 weeks.

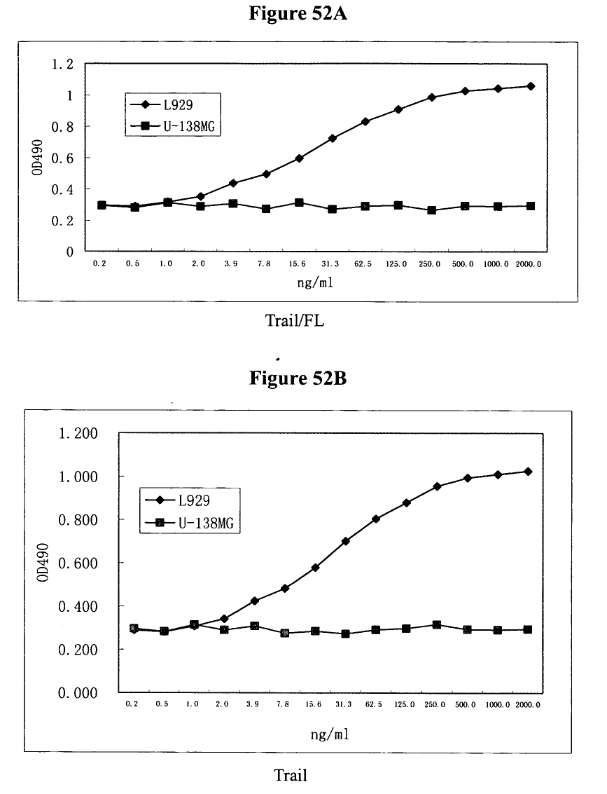

Statistical analysis of the differences was performed using the Student's t test. The results (shown in FIG. 54) indicated that treatment with CD20/FL fusion protein possessed highly significant antitumor activity (p≦0.03).

3. Antitumor activities of Trail/FL in vivo. Human hepatoma cell line QYC was obtained from the International Joint Cancer Institute (Shanghai, China). Female Balb/c nude mice were obtained from Experimental Animal Center (Shanghai, China).

Balb/c nude mice were subcutaneously injected with 1×10⁷ QYC tumor cells. When tumors reached 0.5 cm in diameter, mice were randomized into experimental and control groups with ten mice each. Experimental groups of mice were injected i.p. with Trail/FL at a dose of 10 mg/kg/week for 6 consecutive weeks. The control group of mice were injected i.v. with PBS. Continuous tumor growth was observed in all animals for 6 weeks.

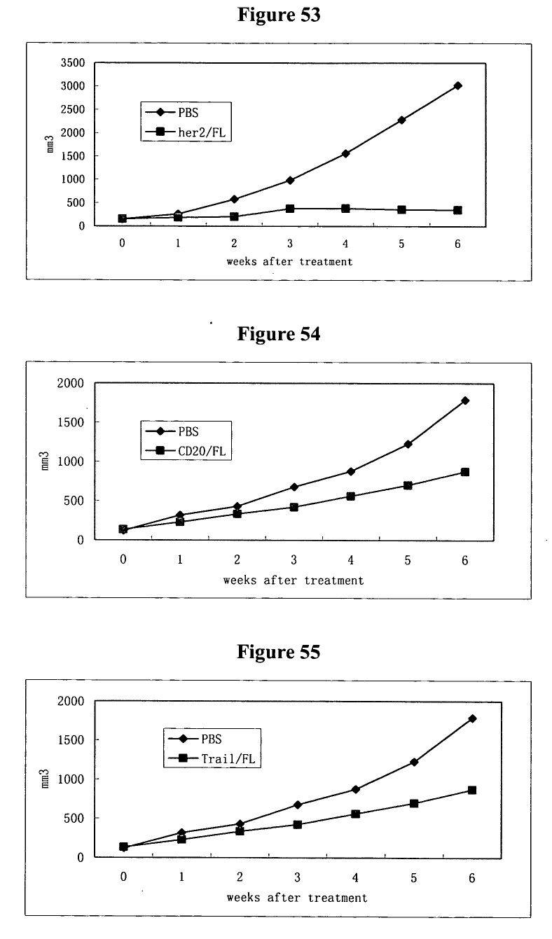

Statistical analysis of the differences was performed using the Student's t test. The results (shown in FIG. 55) indicated that treatment with Trail/FL fusion protein possessed highly significant antitumor activity (p<0.039).

### Example 13

### Specific Tumor Immune Responses Induced by her2/FL CD20/FL and Trail/FL

1. Specific tumor immune responses induced by her2/FL. Mouse breast carcinoma cell lines D2F2, 4T1 of Balb/c origin were obtained from the ATCC. The cell line D2F2/E2 was the cell line D2F2 transfected with human her2 gene. The cell line 4T1her2 was the cell line 4T1 transfected with her2 gene. The her2 antigen was expressed at high levels on the cell surfaces of cell lines D2F2/E2 and 4T1her2. The D2F2/E2 and 4T1her2 tumor cell lines developed subcutaneous tumors in Balb/c mice. The growth of D2F2/E2 and 4T1her2 tumor in mice was effectively inhibited by anti-her2 mAb.

Female Balb/c mice were subcutaneously injected with D2F2, 4T1, D2F2/E2 or 4T1her2 tumor cells. When tumors reached 0.5 cm in diameter, mice inoculated with tumor cells were randomized into five groups with 8 mice each. Mice were injected i.v. with FL, anti-her2 mAb, anti-her2 mAb combined with FL, or huSM/FL at a dose of 4 mg/kg/week for 6 consecutive weeks. The group of mice injected i.v. with PBS was the control group. Continuous tumor growth was observed in all animals for 6 weeks.

The experimental results (shown in table 6) indicated that bifunctional fusion protein her2/FL possessed the ability to inhibit the growth of D2F2/E2 or 4T1her2 comparable to anti-her2 mAb.

Mice bearing regressed D2F2/E2 or 4T1her2 tumor after treatment with fusion proteins or mAb, were challenged again with parental tumor cells subcutaneously. Continuous tumor growth was observed in all animals for 6 weeks. The results (shown in Table 6) indicated that anti-her2 mAb was not effective in inducing active immune response. However, her2/FL elicited active immune response against parental tumor.

TABLE 6
Inhibition of tumor growth by bifunctional fusion proteins

| | Animal | Tumor | | Animal number | |
|---|---|---|---|---|---|
| | number of | regression | | of bearing tumor | Bearing |
| | bearing | after | Cure | after second | tumor |
| treatment | tumor | treatment | rate(%) | challenge | rate(%) |
| PBS | 8 | 0 | 0 | 8 | 100 |
| FL | 16 | 4 | 25 | 14 | 87.5 |
| Anti her2 | 16 | 13 | 81 | 16 | 100 |
| mAb | | | | | |
| Anti her2 | 16 | 14 | 87 | 12 | 75 |
| mAb + FL | | | | | |
| her/FL | 24 | 21 | 87 | 2 | 8 |

Mice bearing regressing D2F2/E2 after treatment with fusion proteins mAb were challenged again with D2F2 or 4T1 tumor cells subcutaneously. Mice bearing regressing 4T1her2 tumors after treatment with fusion proteins were also challenged again with D2F2 or 4T1 tumor cells. Continuous tumor growth was observed in all animals for 6 weeks. The results (shown in Table 6) indicated that D2F2 tumor was rejected in mice in which regression of D2F2/E2 tumor had been induced, while the 4T1 tumor grew progressively. In the other experiment, 4T1 tumor was rejected in mice in which regression of 4T1 her2 tumor had been induced, while D2F2 tumor grew progressively. These results demonstrate that the anti-tumor immune responses induced by bifunctional fusion proteins were tumor-specific.

2. Active tumor immune responses induced by CD20/FL. The cell line A20 was obtained from the ATCC. The cell line A20/CD20 was created by transfecting the D2F2 cell line with the human CD20 gene. The CD20 antigen was expressed at high levels on the cell surfaces of A20/CD20 cells as determined by flow cytometric analysis. The A20/CD20 tumor cell lines developed subcutaneous tumors in Balb/c mice. The growth of A20/CD20 tumor in mice was effectively inhibited by anti-CD20 mAb treatment.

Female Balb/c mice were subcutaneously injected with 2×10⁶ A20/CD20 tumor cells. When tumors reached 0.5 cm in diameter, mice were randomized into groups with 8 mice each. Mice were injected i.v. with FL, anti-CD20 mAb, anti-CD20 mAb combined with FL, or CD20/FL at a dose of 4 mg/kg/week for 6 consecutive weeks. The group of mice injected i.v. with PBS was the negative control group. Continuous tumor growth was observed in all animals for 6 weeks.

The experimental results (shown in table 7) indicated that bifunctional fusion protein CD20/FL possessed the ability to inhibit the growth of A20/CD20 tumor comparable to anti-CD20 mAb treatment.

Mice bearing regressed A20/CD20 tumors after treatment with fusion proteins or mAb, were challenged again with parental tumor cells subcutaneously. Continuous tumor growth was observed in all animals for 6 weeks. The results (shown in Table 7) indicated that anti-CD20 mAb did not induce an active anti-tumor immune response. However, CD20/FL, elicited an active immune response against the parental tumor.

TABLE 7
Induction of active anti-tumor immune response by CD20/FL.

| | | | | Animal number | |
|---|---|---|---|---|---|
| | Animal | Tumor | | of | |
| | number of | regression | | bearing tumor | Bearing |
| | bearing | after | Cure | after second | tumor |
| treatment | tumor | treatment | rate(%) | challenge | rate(%) |
| PBS | 8 | 0 | 0 | 8 | 100 |
| FL | 16 | 4 | 25.0 | 14 | 87.5 |
| Anti CD20 | 12 | 10 | 83.3 | 12 | 100 |
| mAb | | | | | |
| Anti CD20 | 14 | 12 | 85.7 | 10 | 71.4 |
| mAb + FL | | | | | |
| CD20/FL | 20 | 18 | 90.0 | 2 | 10.0 |

3. Active tumor immune responses induced by Trail/FL. The cell line Renca was obtained from the Korea Cancer Institute. Female Balb/c mice were subcutaneously injected with Renca tumor cells. When tumors reached 0.5 cm in diameter, mice were randomized into groups with 8 mice each. Mice were injected i.v. with FL, Trail, Trail combined with FL, or Trail/FL at a dose of 4 mg/kg/week for 6 consecutive weeks. The group of mice injected i.v. with PBS was the control group. Continuous tumor growth was observed in all animals for 6 weeks.

The experimental results (shown in table 8) indicated that bifunctional fusion protein Trail/FL possessed the ability to inhibit the growth of Renca tumor comparable to Trail.

Mice bearing regressing Renca tumors after treatment with fusion proteins or Trail were challenged again with parental tumor cells subcutaneously. Continuous tumor growth was observed in all animals for 6 weeks. The results (shown in Table 8) indicated that Trail did not effectively induce active immune response. However, Trail/FL elicited an active immune response against the parental tumor.

TABLE 8
Induction of active anti-tumor immune response by Trail/FL.

| | | | | Animal number | |
|---|---|---|---|---|---|
| | Animal | Tumor | | of | |
| | number of | regression | | bearing tumor | Bearing |
| | bearing | after | Cure | after second | tumor |
| treatment | tumor | treatment | rate(%) | challenge | rate(%) |
| PBS | 8 | 0 | 0 | 8 | 100 |
| FL | 16 | 5 | 31.3 | 14 | 87.5 |
| Anti CD20 | 14 | 10 | 71.4 | 14 | 100 |
| mAb | | | | | |
| Anti CD20 | 14 | 12 | 85.7 | 10 | 71.4 |
| mAb + FL | | | | | |
| CD20/FL | 18 | 17 | 94.4 | 2 | 11.1 |

In summary, the results demonstrated that the bifunctional fusion proteins not only induce the regression of tumor in vivo, but also elicit a strong active anti-tumor immune response against a subsequent parental tumor challenge.

### Example 14

### Immunohistochemical Analysis of Tumors

In order to further elucidate the mechanism of SM/FL and hSM/FL fusion proteins, immunohistochemistry of tumors was performed on mice treated with the fusion proteins. In these experiments, most tumor cells were killed after administration of chSM/FL and huSM/FL fusion proteins. The tumors were surrounded by an extensive infiltrate of DC, NK, or other lymphocytes, indicating that chSM/FL and huSM/FL fusion proteins induced DC and NK cells to aggregate in tumor tissue and mediated or facilitated tumor cell killing.

1. Inoculation and tumor growth. Hepa/P230 cells were digested with 0.05% trypsin and 0.02% EDTA and adjusted to 2.7×10⁷ cells/ml. The Hepa/P230 cells were subcutaneously inoculated into C57BL/6 mice with 200 ul of tumor cell suspension. When tumors reached 0.5 cm in diameter, mice were injected i.v. with chSM/FL at a dose of 4 mg/kg/week for 3 consecutive weeks. Continuous tumor growth was observed in all animals. Immunohistochemical analysis of tumor samples was performed after treatment.

2. Immunohistochemistry analysis (HE staining). Immunohistochemical analysis via HE staining was performed using standard methods. Briefly, tumor samples were fixed for 24 hours in 10% formalin and embedded in paraffin. Then, 4-μm-thick sections were stained with hematoxylin and eosin.

The results indicated that the administration of FL alone was not significantly effective in killing tumor cells. However, the level of cell killing observed increased when SM5-1 chimeric or humanized mAbs combined with FL. At the same time, some infiltrate of lymphocytes including DC, NK, T cells and B cells was observed in and around tumor tissues. Notably, the SM/FL fusion proteins induced tumor cell lysis in vivo and resulted in an extensive infiltration of lymphocytes into the tumor mass, while the control fusion protein, i.e., (anti-CD3 mAb/FL) did not.

This suggested that the SM/FL fusion proteins had the potent capacity to induce DC, NK and other lymphocytes to aggregate at tumor sites in vivo. The results are shown in Table 9.

TABLE 9
Immunohistochemical analysis of tumors after administration of
chSM/FL and huSM/FL fusion proteins.

| | | results(50 X) | | | |
|---|---|---|---|---|---|
| Treatment | Tumor necrosis | NK | DC | T | B |
| Anti CD3 mAb/FL | + | − | + | + | + |
| FL | + | ++ | ++ | ++ | ++ |
| chSM | +++ | − | − | − | − |
| huSM | ++++ | + | − | − | − |
| chSM + FL | +++ | ++ | ++ | ++ | + |
| huSM + FL | +++ | ++ | ++ | +++ | + |
| chSM/FL | ++++ | ++++ | ++++ | ++++ | ++++ |
| huSM/FL | ++++ | ++++ | ++++ | ++++ | ++++ |

3. Immunohistochemical analysis of tumors after administration of her2/FL, CD20/FL or Trail/FL fusion protein. In order to further elucidate the mechanism of other fusion proteins, immunohistochemical analysis of tumors resected from her2/FL, CD20/FL or Trail/FL fusion protein-treated mice which bearing D2F2/E2, A20/CD20 or Renca was performed as described above. The results are shown in Table 10.

TABLE 10
Immunohistochemical analysis of tumors after administration of
her2/FL, CD20/FL or Trail/FL fusion protein

| | | results(50 X) | | | |
|---|---|---|---|---|---|
| treatment | Tumor necrosis | NK | DC | T | B |
| Anti CD3 mAb/FL | + | − | + | + | + |
| FL | + | ++ | ++ | ++ | ++ |
| Anti HER2 mAb | +++ | − | − | − | − |
| Anti CD20 mAb | ++++ | + | − | − | − |
| TRAIL | ++ | ++ | + | ++ | + |
| Anti Her2 + FL | +++ | ++ | ++ | ++ | + |
| Anti CD20 + FL | +++ | ++ | ++ | +++ | + |
| TRAIL + FL | ++ | +++ | +++ | +++ | +++ |
| Anti Her2/FL | ++++ | +++++ | ++++ | ++++ | ++++ |
| Anti CD20/FL | +++++ | ++++ | ++++ | ++++ | ++++ |
| TRAIL/FL | ++++ | ++++ | ++++ | +++ | ++++ |

The results indicated that chSM/FL, huSM/FL, her2/FL, CD20/FL, and TRAIL/FL fusion proteins inhibited tumor cell growth by recruiting and activating. The fusion proteins induced NK and DC cells to aggregate at tumor sites, and DC, NK and other lymphocytes exerted their antitumor activities.

### Example 15

### In Vivo Biodistribution of Fusion Proteins

To study the specific binding of chSM/FL or huSM/FL to tumor cells, the biodistribution characteristics of fusion proteins were examined.

The mice bearing B16p230 tumor were injected i.v. with ¹²⁵I-labeled chSM, chSM/FL, huSM and huSM/FL individually. After 48 h, selected organs were immediately removed and radioactivity was determined.

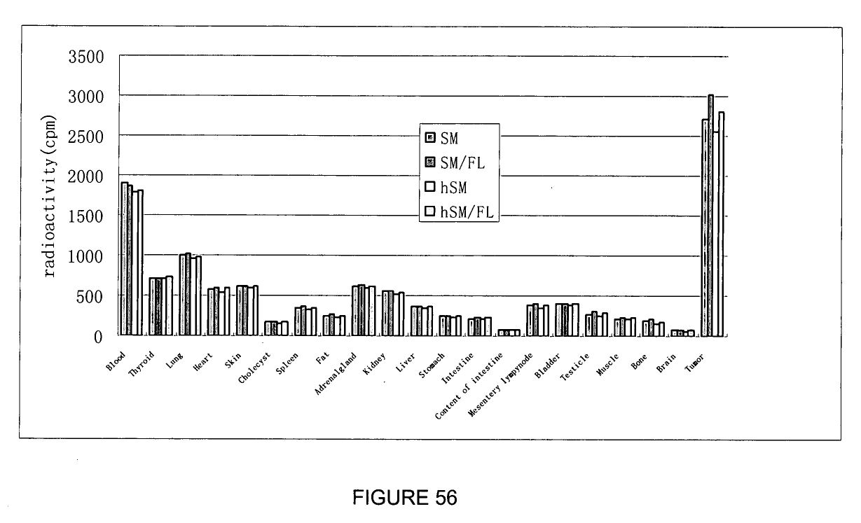

The results (shown in FIG. 56) indicated that the biodistribution of chSM/FL and hSM/FL fusion proteins were similar to that of chimeric mAb chSM or humanized mAb huSM. The fusion proteins all retained the specificity of their parental mAbs and were highly concentrated at tumor sites.

The biodistribution of the mAbs and fusion proteins depended on their specificity, a significant factor in clinical applications. The specific tissue distribution reduces the dose of drugs required to achieve the desired effect; as well as reducing the damage to non-targeted tissues.

The in vivo distribution characteristics of her2/FL, CD20/FL and TRAIL/FL fusion proteins were also examined. The mice bearing 4T1/her2, A20/20 and Renca tumor were injected i.v. with ¹²⁵I labeled her2/FL, CD20/FL and TRAIL/FL and huSM/FL, respectively. After 48 h, selected organs were immediately removed and radioactivity was determined.

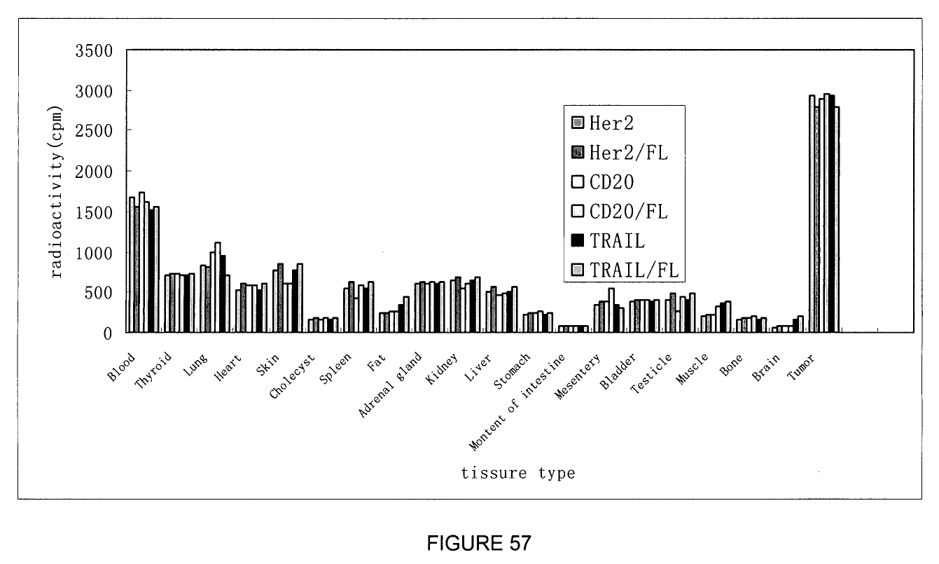

The study results (shown in FIG. 57) indicated that her2/FL, CD20/FL and TRAIL/FL fusion proteins localized at the tumor sites, similar to chSM/FL and hSM/FL.

### Example 16

### Adoptive Immunotherapy with Tumor-Specific Lymphocytes

HepaP230 or B16p230 cells were digested with 0.05% trypsin and 0.02% EDTA and adjusted to 2.7×10⁷ cells/ml. The Hepa1-6/P230 or B16/P230 cells were subcutaneously inoculated into C57BL/6 mice with 200 ul of tumor cell suspension. When tumors reached 0.5 cm in diameter, mice were injected i.v. with chSM/FL at a dose of 4 mg/kg/week for 3 consecutive weeks. Continuous tumor growth was observed in all animals.

Mice treated with fusion proteins chSM/FL or huSM/FL and in which regression of the tumor hepap230 or B16p230 had occurred were sacrificed and spleens were harvested. Spleen cells were isolated and adjusted to 1.0×10⁹ cells/ml. Then, naive mice were injected with 5.0×10⁷ spleen cells from mice in which regression of hepap230 or B16p230 tumor had occurred and challenged with hepap230 or B16p230 tumors, respectively. Continuous tumor growth was observed in all animals for 6 weeks.

The results (shown in Table 11) indicated that mice adopting spleen cells from mice spleen cells treated with fusion proteins chSM/FL or huSM/FL and in which regression of the tumor hepap230 or B16p230 occurred rejected the parental tumor. The transfer of spleen cells from mice treated with non fusion protein combinations, i.e., chSM, huSM, FL, chSM combined with FL or huSM combined with FL, failed to induce tumor rejection in recipient mice. These results suggested that the transferred lymphocytes mounted a specific anti-tumor immune response, and the specific immune response was facilitated by DC and NK cells.

TABLE 11
Adoptive immunotherapy with tumor-specific lymphocytes.

| | | Mortality after | |
|---|---|---|---|
| Treatment of | | transfusion | |
| Spleen cell donor | Recipient number | Hepap230 | B16p230 |
| Anti CD3 mAb/FL | 15 | 15/15 | 15/15 |
| FL | 15 | 9/15 | 10/15 |
| chSM | 15 | 12/15 | 14/15 |
| huSM | 15 | 13/15 | 14/15 |
| chSM + FL | 15 | 10/15 | 10/15 |
| huSM + FL | 15 | 10/15 | 10/15 |
| SM/FL | 15 | 0/15 | 1/15 |
| hSM/FL | 15 | 1/15 | 0/15 |

The results also indicated that the antitumor mechanism of chSM/FL and huSM/FL fusion proteins depended on specific active tumor immune responses.

T1/her2, A20/20 and Renca cells were digested with 0.05% trypsin and 0.02% EDTA and adjusted to 2.7×10⁷ cells/ml. The 4T1/her2, A20/20 or Renca cells were subcutaneously inoculated into mice with 200 ul of tumor cell suspension. When tumors reached 0.5 cm in diameter, mice were injected i.v. with her2/FL, CD20/FL or Trail/FL at a dose of 4 mg/kg/week for 3 consecutive weeks. Continuous tumor growth was observed in all animals.

Mice treated with fusion proteins her2/FL, CD20/FL or Trail/FL and in which regression of the tumor 4T1/her2, A20/20 or Renca cells had occurred were sacrificed and spleens were harvested. Spleen cells were isolated and adjusted to 1.0×10⁹ cells/ml. Then, naive mice were injected with 5.0×10⁷ spleen cells from mice in which regression of T1/her2, A20/20 or Renca tumor had occurred and then challenged with 4T1/her2, A20/20 or Renca tumors, respectively. Continuous tumor growth was observed in all animals for 6 weeks.

The results (shown in Table 12) indicated that mice adopting spleen cells from mice spleen cells treated with fusion proteins chSM/FL or huSM/FL and in which regression of the tumor hepap230 or B16p230 occurred rejected the parental tumor.

The results (shown in Table 12) are consistent with that of chSM/FL and huSM/FL, indicating chSM/FL, huSM/FL, her2/FL, CD20/FL and Trail/FL medicated anti-tumor activity by activating lymphocytes.

TABLE 12
Anti-tumor activity by activating lymphocytes.

| Treatment of | Recipient | Mortality after transfusion |
|---|---|---|
| Spleen cell donor | number | Cell line* |
| Anti CD3 mAb/FL | 15 | 15/15 |
| FL | 15 | 9/15 |
| Anti Her2 mAb | 15 | 11/15 |
| Anti Her mAb + FL | 15 | 9/15 |
| HER2/FL | 15 | 4/15 |
| Anti CD20 mAb | 15 | 13/15 |
| Anti CD20 mAb + FL | 15 | 10/15 |
| CD20/FL | 15 | 2/15 |
| TRAIL | 15 | 8/15 |
| TRAIL + FL | 15 | 10/15 |
| TRAIL/FL | 15 | 5/15 |

*cell line: 4T1/her2, A20/20 and Renca cell lines were used in Her2, CD20, TRAIL related experiments, respectively.

Many modifications and variations of this invention can be made without departing from its spirit and scope, as will be apparent to those skilled in the art. The specific embodiments described herein are offered by way of example only, and the invention is to be limited by the terms of the appended claims, along with the full scope of equivalents to which such claims are entitled; and the invention is not to be limited by the specific embodiments that have been presented herein by way of example.

Citation of the above publications or documents is not intended as an admission that any of the foregoing is pertinent prior art, nor does it constitute any admission as to the contents or date of these publications or documents. U.S. patents and other publications referenced herein are hereby incorporated by reference.

1. An isolated chimeric protein, which chimeric protein comprises a Flt3 ligand, or a biologically active fragment thereof, and a proteinaceous or peptidyl tumoricidal agent.

2. The chimeric protein of claim 1, wherein the tumoricidal agent induces apoptosis.

3. The chimeric protein of claim 1, wherein the Flt3 ligand, or a biologically active fragment thereof, stimulates the proliferation of hematopoietic stem or progenitor cells.

4. The chimeric protein of claim 1, wherein the Flt3 ligand, or a biologically active fragment thereof, stimulates the proliferation of cells selected from the group consisting of myeloid precursor cells, monocytic cells, macrophages, B-cells, dendritic cells and NK cells.

5. The chimeric protein of claim 1, wherein the Flt3 ligand, or a biologically active fragment thereof, is a mammalian Flt3-ligand.

6. The chimeric protein of claim 5, wherein the mammalian Flt3 ligand, or a biologically active fragment thereof, is a human Flt3 ligand.

7. The chimeric protein of claim 1, wherein the Flt3 ligand, or a biologically active fragment thereof, is a soluble Flt3 ligand.

8. The chimeric protein of claim 1, wherein the Flt3 ligand comprises at least 100 amino acid residues and the Flt3 ligand has at least 40% identity to the amino acid sequence set forth in SEQ ID NO:2, in which the percentage identity is determined over an amino acid sequence of identical size to the amino acid sequence set forth in SEQ ID NO:2, and the Flt3 ligand substantially retains its biological activity.

9. The chimeric protein of claim 1, wherein the Flt3 ligand binds to an antibody that specifically binds to an amino acid sequence set forth in SEQ ID NO:2 and the Flt3 ligand substantially retains its biological activity.

10. The chimeric protein of claim 1, wherein the Flt3 ligand comprises the amino acid sequence set forth in SEQ ID NO:2.

11. The chimeric protein of claim 1, wherein the Flt3 ligand comprises an amino acid sequence that is at least 80% identical to amino acids 28 to 128 of SEQ ID NO:2.

12. The chimeric protein of claim 1, wherein the Flt3 ligand comprises amino acids 28 to 128 of SEQ ID NO:2.

13. The chimeric protein of claim 1, wherein the Flt3 ligand comprises an amino acid sequence selected from the group consisting of amino acid residues 28-160 of SEQ ID NO:2, and amino acid residues 28-182 of SEQ ID NO:2.

14. The chimeric protein of claim 1, wherein the tumoricidal agent is an antibody.

15. The chimeric protein of claim 14, wherein the antibody is selected from the group consisting of an intact antibody, a Fab fragment, a Fab′ fragment, a F(ab′)₂ fragment, a Fv fragment, a diabody, a single-chain antibody and a multi-specific antibody formed from antibody fragments.

16. The chimeric protein of claim 14, wherein the antibody is selected from the group consisting of an anti-p230 antibody, an anti-CD20 antibody, an anti-Her2 antibody, an anti-Her3 antibody, an anti-Her4 antibody, an anti-EGFR antibody or a fragment thereof that retains binding activity for the target antigen of the antibody.

17. The chimeric protein of claim 14, wherein the antibody is a human or humanized antibody.

18. The chimeric protein of claim 1, wherein the tumoricidal agent is selected from the group consisting of Fas ligand, TNF, TRAIL, or a biologically active extracellular domain thereof.

19. The chimeric protein of claim 1, wherein the tumoricidal agent is other than TRAIL.

20. The chimeric protein of claim 1, wherein the Flt3 ligand, or a biologically active fragment thereof, is located at the N-terminus of the chimeric protein.

21. The chimeric protein of claim 1, wherein the Flt3 ligand, or a biologically active fragment thereof, is located at the C-terminus of the chimeric protein.

22. The chimeric protein of claim 1, wherein the Flt3 ligand, or a biologically active fragment thereof, and the tumoricidal are separated by a linking peptide.

23. The chimeric protein of claim 22, wherein the linking peptide is (Gly₄Ser)₃.

24. The chimeric protein of claim 1, which comprises the amino acid sequence set forth in SEQ ID NO:24, SEQ ID NO:26, SEQ ID NO:28, SEQ ID NO:30, SEQ ID NO:32, SEQ ID NO:34, SEQ ID NO:44, SEQ ID NO:46, SEQ ID NO:48, SEQ ID NO:58, SEQ ID NO:60, SEQ ID NO:62, SEQ ID NO:64, SEQ ID NO:66 or SEQ ID NO:68.

25. An isolated nucleic acid comprising a nucleotide sequence encoding a chimeric protein comprising a Flt3 ligand, or a biologically active fragment thereof, and a proteinaceous or peptidyl tumoricidal agent other than TRAIL.

26. The nucleic acid of claim 25, which comprises the nucleotide sequence set forth in SEQ ID NO:23, SEQ ID NO:25, SEQ ID NO:27, SEQ ID NO:29, SEQ ID NO:31, SEQ ID NO:33, SEQ ID NO:43, SEQ ID NO:45, SEQ ID NO:47, SEQ ID NO:57, SEQ ID NO:59, SEQ ID NO:61, SEQ ID NO:63, SEQ ID NO:65 or SEQ ID NO:67.

27. An isolated nucleic acid comprising a nucleotide sequence complementary to the nucleotide sequence of claim 25.

28. A vector comprising the nucleotide sequence of claim 25.

29. The vector of claim 28, which further comprises a regulatory sequence operatively linked to the nucleic acid encoding the Flt3 ligand, or a biologically active fragment thereof, and the proteinaceous or peptidyl tumoricidal agent.

30. A recombinant cell containing the nucleic acid of claim 25.

31. The recombinant cell of claim 30, which is an eukaryotic cell.

32. The recombinant cell of claim 31, which is a CHO, COS, or NSO cell.

33. A method of producing a chimeric protein comprising growing a recombinant cell containing the nucleic acid of claim 25 such that the encoded chimeric protein is expressed by the cell, and recovering the expressed chimeric protein.

34. The method of claim 33, which further comprises isolating and/or purifing the recovered chimeric protein.

35. The product of the method of claim 33.

36. A pharmaceutical composition comprising an effective amount of a chimeric protein comprising a Flt3 ligand, or a biologically active fragment thereof, and a proteinaceous or peptidyl tumoricidal agent, and a pharmaceutically acceptable carrier or excipient.

37. A kit comprising an effective amount of a chimeric protein comprising a Flt3 ligand, or a biologically active fragment thereof, and a proteinaceous or peptidyl tumoricidal agent, and instructions for administering said chimeric protein.

38. A method for treating cancer in a mammal so affected, which method comprises administering to the mammal an effective amount of an isolated chimeric protein comprising a Flt3 ligand, or a biologically active fragment thereof, and a proteinacuous or peptidyl tumoricidal agent, wherein the cancer expresses a target for the proteinaceous or peptidyl tumoricidal agent.

39. The method of claim 38, wherein the mammal is a human.

40. The method of claim 38, wherein the cancer is melanoma, breast cancer or hepatocellular carcinoma.

41. A combination, which combination comprises:
a) an effective amount of a chimeric protein comprising a Flt3 ligand, or a biologically active fragment thereof, and a proteinaceous or peptidyl tumoricidal agent; and
b) an effective amount of an anti-neoplastic agent.

42. The combination of claim 41, wherein the anti-neoplastic agent is an agent that treats melanoma, breast cancer or hepatocellular carcinoma.

43. A method for treating cancer in a mammal so afflicted, which method comprises administering to the mammal an effective amount of a combination of claim 40 wherein the cancer expresses a target for the proteinaceous or peptidyl tumoricidal agent.

44. A method for inducing caspase-3 mediated apoptosis in a cell, which method comprises contacting the cell with an effective amount of an isolated chimeric protein comprising an isolated Flt3 ligand, or a biologically active fragment thereof, and a proteinaceous or peptidyl tumoricidal agent, wherein the cell expresses a target for the proteinaceous or peptidyl tumoricidal agent.

45. The method of claim 44, wherein the cell is a mammalian cell.

46. The method of claim 45, wherein the cell is a mammalian neoplasm cell.

47. The method of claim 44, wherein the cell is contained in a mammal.

48. A vaccine comprising an effective amount of a chimeric protein comprising a Flt3 ligand, or a biologically active fragment thereof, and a proteinaceous or peptidyl tumoricidal agent and an immune response potentiator.

49. The vaccine of claim 48, wherein the immune response potentiator is other than flt3 ligand.

50. A method for eliciting an anti-cancer immune response in a mammal so afflicted, which method comprises administering to the mammal an effective amount of a vaccine of claim 48.

51. A method for producing a tumor-specific lymphocyte, which method comprises administering to a mammal an effective amount of an isolated chimeric protein comprising a Flt3 ligand, or a biologically active fragment thereof, and a proteinaceous or peptidyl tumoricidal agent to generate a tumor-specific lymphocyte, and recovering said generated tumor-specific lymphocyte from said mammal.

52. An isolated chimeric protein, which chimeric protein comprises a Flt3 ligand, or a biologically active fragment thereof, and a proteinaceous or peptidyl targeting agent which binds to a receptor expressed on tumor cells other than the Fc receptor.

53. The chimeric protein of claim 52, wherein the tumoricidal agent induces apoptosis.

54. The chimeric protein of claim 52, wherein the Flt3 ligand, or a biologically active fragment thereof, stimulates the proliferation of hematopoietic stem or progenitor cells.

55. The chimeric protein of claim 52, wherein the Flt3 ligand, or a biologically active fragment thereof, stimulates the proliferation of cells selected from the group consisting of myeloid precursor cells, monocytic cells, macrophages, B-cells, dendritic cells and NK cells.

56. The chimeric protein of claim 52, wherein the Flt3 ligand, or a biologically active fragment thereof, is a mammalian Flt3-ligand.

57. The chimeric protein of claim 56, wherein the mammalian Flt3 ligand, or a biologically active fragment thereof, is a human Flt3 ligand.

58. The chimeric protein of claim 52, wherein the Flt3 ligand, or a biologically active fragment thereof, is a soluble Flt3 ligand.

59. The chimeric protein of claim 1, wherein the Flt3 ligand comprises at least 100 amino acid residues and the Flt3 ligand has at least 40% identity to the amino acid sequence set forth in SEQ ID NO:2, in which the percentage identity is determined over an amino acid sequence of identical size to the amino acid sequence set forth in SEQ ID NO:2, and the Flt3 ligand substantially retains its biological activity.

60. The chimeric protein of claim 52, wherein the Flt3 ligand binds to an antibody that specifically binds to an amino acid sequence set forth in SEQ ID NO:2 and the Flt3 ligand substantially retains its biological activity.

61. The chimeric protein of claim 52, wherein the Flt3 ligand comprises the amino acid sequence set forth in SEQ ID NO:2.

62. The chimeric protein of claim 52, wherein the Flt3 ligand comprises an amino acid sequence that is at least 80% identical to amino acids 28 to 128 of SEQ ID NO:2.

63. The chimeric protein of claim 52, wherein the Flt3 ligand comprises amino acids 28 to 128 of SEQ ID NO:2.

64. The chimeric protein of claim 52, wherein the Flt3 ligand comprises an amino acid sequence selected from the group consisting of amino acid residues 28-160 of SEQ ID NO:2, and amino acid residues 28-182 of SEQ ID NO:2.

65. The chimeric protein of claim 52, wherein the targeting agent which binds to a receptor expressed on tumor cells is an antibody.

66. The chimeric protein of claim 65, wherein the antibody is selected from the group consisting of an intact antibody, a Fab fragment, a Fab′ fragment, a F(ab′)₂ fragment, a Fv fragment, a diabody, a single-chain antibody and a multi-specific antibody formed from antibody fragments.

67. The chimeric protein of claim 52, wherein the antibody is a human or humanized antibody.

68. The chimeric protein of claim 52, wherein the Flt3 ligand, or a biologically active fragment thereof, is located at the N-terminus of the chimeric protein.

69. The chimeric protein of claim 52, wherein the Flt3 ligand, or a biologically active fragment thereof, is located at the C-terminus of the chimeric protein.

70. The chimeric protein of claim 52, wherein the Flt3 ligand, or a biologically active fragment thereof, and the tumoricidal are separated by a linking peptide.

71. The chimeric protein of claim 70, wherein the linking peptide is (Gly₄Ser)₃.

72. The chimeric protein of claim 52 wherein the receptor expressed on tumor cells is not the E6 or E7 proteins human papilloma virus.

73. The chimeric protein of claim 52, wherein the receptor expressed on tumor cells is not a receptor for TRAIL.

74. An isolated nucleic acid comprising a nucleotide sequence complementary to the nucleotide sequence of claim 52.

75. A vector comprising the nucleotide sequence of claim 74.

76. The vector of claim 75, which further comprises a regulatory sequence operatively linked to the nucleic acid encoding the Flt3 ligand, or a biologically active fragment thereof, and the proteinaceous or peptidyl tumoricidal agent.

77. A recombinant cell containing the nucleic acid of claim 74.

78. The recombinant cell of claim 77, which is an eukaryotic cell.

79. The recombinant cell of claim 77, which is a CHO, COS, or NSO cell.

80. A method of producing a chimeric protein comprising growing a recombinant cell containing the nucleic acid of claim 74 such that the encoded chimeric protein is expressed by the cell, and recovering the expressed chimeric protein.

81. The method of claim 80, which further comprises isolating and/or purifing the recovered chimeric protein.

82. The product of the method of claim 80.

83. A pharmaceutical composition comprising an effective amount of a chimeric protein comprising a Flt3 ligand, or a biologically active fragment thereof, and a targeting agent which binds to a receptor expressed on tumor cells other than the Fc receptor, and a pharmaceutically acceptable carrier or excipient.

84. A kit comprising an effective amount of a chimeric protein comprising a Flt3 ligand, or a biologically active fragment thereof, and a targeting agent which binds to a receptor expressed on tumor cells other than the Fc receptor, and instructions for administering said chimeric protein.

85. A method for treating cancer in a mammal so affected, which method comprises administering to the mammal an effective amount of an isolated chimeric protein comprising a Flt3 ligand, or a biologically active fragment thereof, and a targeting agent which binds to a receptor expressed on tumor cells other than the Fc receptor, wherein the cancer expresses a receptor for the targeting agent.

86. The method of claim 85, wherein the mammal is a human.

87. The method of claim 85, wherein the cancer is melanoma, breast cancer or hepatocellular carcinoma.

88. A combination, which combination comprises:
a) an effective amount of a chimeric protein comprising a Flt3 ligand, or a biologically active fragment thereof, and a targeting agent which binds to a receptor expressed on tumor cells other than the Fc receptor; and
b) an effective amount of an anti-neoplastic agent.

89. The combination of claim 88, wherein the anti-neoplastic agent is an agent that treats melanoma, breast cancer or hepatocellular carcinoma.

90. A method for treating cancer in a mammal so afflicted, which method comprises administering to the mammal an effective amount of a combination of claim 89 wherein the cancer expresses a receptor for the targeting agent.

91. A method for inducing caspase-3 mediated apoptosis in a cell, which method comprises contacting the cell with an effective amount of an isolated chimeric protein comprising an isolated Flt3 ligand, or a biologically active fragment thereof, and a targeting agent which binds to a receptor expressed on tumor cells other than the Fc receptor, wherein the cell expresses a receptor for the targeting agent.

92. The method of claim 91, wherein the cell is a mammalian cell.

93. The method of claim 91, wherein the cell is a mammalian neoplasm cell.

94. The method of claim 91, wherein the cell is contained in a mammal.

95. A vaccine comprising an effective amount of a chimeric protein comprising a Flt3 ligand, or a biologically active fragment thereof, and and a targeting agent which binds to a receptor expressed on tumor cells other than the Fc receptor, and an immune response potentiator.

96. The vaccine of claim 95, wherein the immune response potentiator is other than flt3 ligand.

97. A method for eliciting an anti-cancer immune response in a mammal so afflicted, which method comprises administering to the mammal an effective amount of a vaccine of claim 95.

98. A method for producing a tumor-specific lymphocyte, which method comprises administering to a mammal an effective amount of an isolated chimeric protein comprising a Flt3 ligand, or a biologically active fragment thereof, and a targeting agent which binds to a receptor expressed on tumor cells other than the Fc receptor, to generate a tumor-specific lymphocyte, and recovering said generated tumor-specific lymphocyte from said mammal.
